(19) United States
(12) Patent Application Publication
(10) Pub. No.: US 2002/0052370 A1
(43) Pub. Date: May 2, 2002

(54) Cyclopentyl-substituted glutaramide derivatives as inhibitors of neutral endopeptidase

(76) Inventors: Christopher Gordon Barber, Kent (GB); Andrew Simon Cook, Kent (GB); Graham Nigel Maw, Kent (GB); David Cameron Pryde, Kent (GB); Alan Stobie, Kent (GB)

Correspondence Address: Gregg C. Benson, Pfizer Inc., Patent Department, MS 4159, Eastern Point Road, Groton, CT, 06340, US

(21) Appl. No.: 09/893,585

(22) Filed: Jun. 28, 2001

Related U.S. Application Data
(60) Provisional application No. 60/219,100, filed on Jul. 18, 2000.
(60) Provisional application No. 60/274,957, filed on Mar. 12, 2001.

(30) Foreign Application Priority Data
Jul. 6, 2000 (GB) 0016684.3
Jan. 22, 2001 (GB) 0101584.1

(51) Int. Cl. A61K 31/535; A61K 31/495; A61K 31/4453; A61K 31/40; A61K 31/195

(52) U.S. Cl. 514/237.5; 514/255.01; 514/317; 514/423; 514/563

(57) ABSTRACT

The invention provides compounds of formula I wherein R¹ is optionally substituted C₁₋₆alkyl, optionally substituted C₃₋₇cycloalkyl, optionally substituted aryl or optionally substituted heterocyclyl; n is 0, 1 or 2; and Y is —NR¹⁸S(O)ᵤR¹⁹ or a group shown below.

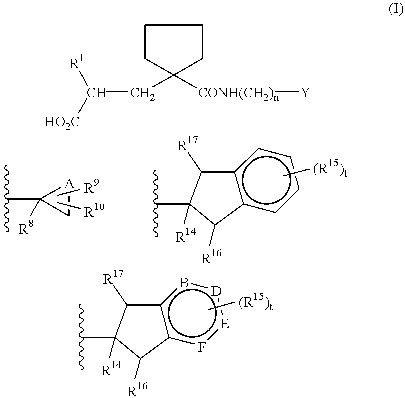

### CROSS REFERENCE TO RELATED APPLICATIONS

This application claims benefit of the filing of U.S. Ser. No. 60/219,100 filed Jul. 18, 2000 and U.S. Ser. No. 60/274,957 filed Mar. 12, 2001 under 37 C.F.R. 1.78 (a) 3.

### FIELD OF THE INVENTION

This invention relates to inhibitors of neutral endopeptidase enzyme (NEP), uses thereof, processes for the preparation thereof, intermediates used in the preparation thereof, and compositions containing said inhibitors. These inhibitors have utility in a variety of therapeutic areas including the treatment of female sexual dysfunction (FSD) especially female sexual arousal disorder (FSAD).

### BRIEF SUMMARY OF THE INVENTION

In a first aspect, the invention relates to a method of treating female sexual dysfunction comprising administering a therapeutically effective amount of a compound of formula (I), and/or its pharmaceutically acceptable salts, solvates, polymorphs or prodrugs thereof

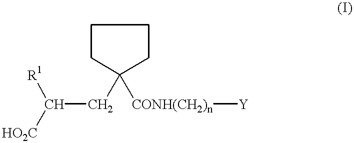

wherein

R¹ is C₁₋₆alkyl which may be substituted by one or more substituents, which may be the same or different, selected from the list: halo, hydroxy, C₁₋₆alkoxy, C₁₋₆hydroxyalkoxy, C₁₋₆alkoxy(C₁₋₆alkoxy), C₃₋₇cycloalkyl, C₃₋₇cycloalkenyl, aryl, aryloxy, (C₁₋₄alkoxy)aryloxy, heterocyclyl, heterocyclyloxy, —NR²R³, —NR⁴COR⁵, —NR⁴SO₂R⁵, —CONR²R³, —S(O)ₚR⁶, —COR⁷ and —CO₂(C₁₋₄alkyl); or R¹ is C₃₋₇cycloalkyl, aryl or heterocyclyl, each of which may be substituted by one or more heterocyclyl, each of which may be substituted by one or more substituents from said list, which substituents may be the same or different, which list further includes C₁₋₆alkyl; or R¹ is C₁₋₆alkoxy, —NR²R³ or —NR⁴SO₂R⁵;

wherein

R² and R³ are each independently H, C₁₋₄alkyl, C₃₋₇cycloalkyl (optionally substituted by hydroxy or C₁₋₄alkoxy), aryl, (C₁₋₄alkyl)aryl, C₁₋₆alkoxyaryl or heterocyclyl; or R² and R³ together with the nitrogen to which they are attached form a pyrrolidinyl, piperidino, morpholino, piperazinyl or N—(C₁₋₄alkyl)piperazinyl group;

R⁴ is H or C₁₋₄alkyl;

R⁵ is C₁₋₄alkyl, CF₃, aryl, (C₁₋₄alkyl)aryl, (C₁₋₄alkoxy)aryl, heterocyclyl, C₁₋₄alkoxy or —NR²R³ wherein R² and R³ are as previously defined;

R⁶ is C₁₋₄alkyl, aryl, heterocyclyl or NR²R³ wherein R² and R³ are as previously defined; and

R⁷ is C₁₋₄alkyl, C₃₋₇cycloalkyl, aryl or heterocyclyl; p is 0, 1, 2 or 3;

n is 0, 1 or 2;

the —(CH₂ )ₙ— linkage is optionally substituted by C₁₋₄alkyl, C₁₋₄alkyl substituted with one or more fluoro groups or phenyl, C₁₋₄alkoxy, hydroxy, hydroxy(C₁₋₃alkyl), C₃₋₇cycloalkyl, aryl or heterocyclyl;

Y is the group

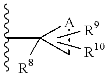

wherein A is —(CH₂)_{q}— where q is 1, 2, 3 or 4 to complete a 3 to 7 membered carbocyclic ring which may be saturated or unsaturated; R⁸ is H, C₁₋₆alkyl, —CH₂OH, phenyl, phenyl(C₁₋₄alkyl) or CONR¹¹R¹²; R⁹ and R¹⁰ are each independently H, —CH₂OH, —C(O)NR¹¹R¹², C₁₋₆alkyl, phenyl (optionally substituted by C₁₋₄alkyl, halo or C₁₋₄alkoxy or phenyl(C₁₋₄alkyl) wherein the phenyl group is optionally substituted by C₁₋₄alkyl, halo or C₁₋₄alkoxy, or R⁹ and R¹⁰ together form a dioxolane; R¹¹ and R¹² which may be the same or different are H, C₁₋₄alkyl, R¹³ or S(O)ᵣR¹³, where r is 0, 1 or 2 and R¹³ is phenyl optionally substituted by C₁₋₄alkyl or phenyl C₁₋₄alkyl wherein the phenyl is optionally substituted by C₁₋₄alkyl; or

Y is the group, —C(O)NR¹¹R¹² wherein R¹¹ and R¹² are as previously defined except that R¹¹ and R¹² are not both H; or

Y is the group,

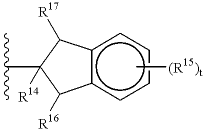

wherein R¹⁴ is H, CH₂OH, or C(O)NR¹¹R¹² wherein R¹¹ and R¹² are as previously defined; when present R¹⁵, which may be the same or different to any other R¹⁵, is OH, C₁₋₄alkyl, C₁₋₄alkoxy, halo or CF₃; t is 0, 1, 2, 3 or 4; and R¹⁶ and R¹⁷ are independently H or C₁₋₄alkyl; or

Y is the group

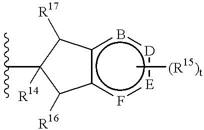

wherein one or two of B, D, E or F is a nitrogen, the others being carbon; and R¹⁴ to R¹⁷ and t are as previously defined; or

Y is an optionally substituted 5-7 membered heterocyclic ring, which may be saturated, unsaturated or aromatic and contains a nitrogen, oxygen or sulphur and optionally one, two or three further nitrogen atoms in the ring and which may be optionally benzofused and optionally substituted by:

C₁₋₆alkoxy; hydroxy; oxo; amino; mono or di-(C₁₋₄alkyl)amino; C₁₋₄alkanoylamino; or

C₁₋₆alkyl which may be substituted by one or more substituents, which may be the same or different, selected from the list: C₁₋₆alkoxy, C₁₋₆haloalkoxy, C₁₋₆alkylthio, halogen, C₃₋₇cycloalkyl, heterocyclyl or phenyl; or

C₃₋₇cycloalkyl, aryl or heterocyclyl, each of which may be substituted by one or more substituents, which may be the same or different, selected from the list: C₁₋₆alkyl, C₁₋₆alkoxy, C₁₋₆haloalkoxy, C₁₋₆alkylthio, halogen, C₃₋₇cycloalkyl, heterocyclyl or phenyl;

wherein when there is an oxo substitution on the heterocyclic ring, the ring only contains one or two nitrogen atoms and the oxo substitution is adjacent a nitrogen atom in the ring; or

Y is —NR¹⁸S(O)ᵤR¹⁹, wherein R¹⁸ is H or C₁₋₄alkyl; R¹⁹ is aryl, aryl C₁₋₄alkyl or heterocyclyl; and u is 0, 1, 2 or 3.

The invention further relates to a compound of formula (I), pharmaceutically acceptable salts, solvates, polymorphs or prodrugs thereof, wherein R¹, n and Y are as defined in the first aspect with the proviso that Y is not the group —C(O)NR¹¹R¹² and when R¹ is propyl or phenylethyl, R¹⁴ is not —CH₂OH.

In a third aspect, the invention relates to a compound of formula (I), pharmaceutically acceptable salts, solvates, polymorphs or prodrugs thereof, wherein R¹, n and Y are as defined in the first aspect with the proviso that Y is not the group —C(O)NR¹¹R¹² and R¹⁴ is not H or —CH₂OH.

### BRIEF SUMMARY OF THE DRAWINGS

FIG. 1 shows the effect on vaginal and clitoral blood flow of intravenous administration of a compound of the invention.

FIG. 2 shows the effect on vaginal blood flow over time of administering a compounds of the invention topically

### DETAILED DESCRIPTION OF THE INVENTION

According to a first aspect, the invention provides the use of a compound of formula (I), pharmaceutically acceptable salts, solvates, polymorphs or prodrugs thereof, in the preparation of a medicament for the treatment of female sexual dysfunction;

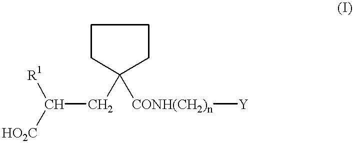

wherein

R¹ is C₁₋₆alkyl which may be substituted by one or more substituents, which may be the same or different, selected from the list: halo, hydroxy, C₁₋₆alkoxy, C₂₋₆hydroxyalkoxy, C₁₋₆alkoxy(C₁₋₆alkoxy), C₃₋₇cycloalkyl, C₃₋₇cycloalkenyl, aryl, aryloxy, (C₁₋₄alkoxy)aryloxy, heterocyclyl, heterocyclyloxy, —NR²R³, —NR⁴COR⁵, —NR⁴SO₂R⁵, —CONR²R³, —S(O)ₚR⁶, —COR⁷ and —CO₂(C₁₋₄alkyl); or R¹ is C₃₋₇cycloalkyl, aryl or heterocyclyl, each of which may be substituted by one or more substituents from said list, which substituents may be the same or different, which list further includes C₁₋₆alkyl; or R¹ is C₁₋₆alkoxy, —NR² R³ or —NR⁴SO₂R⁵;

wherein

R² and R³ are each independently H, C₁₋₄alkyl, C₃₋₇cycloalkyl (optionally substituted by hydroxy or C₁₋₄alkoxy), aryl, (C₁₋₄alkyl)aryl, C₁₋₆alkoxyaryl or heterocyclyl; or R² and R³ together with the nitrogen to which they are attached form a pyrrolidinyl, piperidino, morpholino, piperazinyl or N—(C₁₋₄alkyl)piperazinyl group;

R⁴ is H or C₁₋₄alkyl;

R⁵ is C₁₋₄alkyl, CF₃, aryl, (C₁₋₄ alkyl)aryl, (Cl 4alkoxy)aryl, heterocyclyl, C₁₋₄alkoxy or —NR²R³ wherein R² and R³ are as previously defined;

R⁶ is C₁₋₄alkyl, aryl, heterocyclyl or NR²R³ wherein R² and R³ are as previously defined; and

R⁷ is C₁₋₄alkyl, C₃₋₇cycloalkyl, aryl or heterocyclyl; p is 0, 1, 2 or 3;

n is 0, 1 or 2;

the —(CH₂)ₙ— linkage is optionally substituted by C₁₋₄alkyl, C₁₋₄alkyl substituted with one or more fluoro groups or phenyl, C₁₋₄alkoxy, hydroxy, hydroxy(C₁₋₃alkyl), C₃₋₇cycloalkyl, aryl or heterocyclyl;

Y is the group

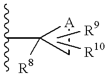

wherein A is —(CH₂)₁— where q is 1, 2, 3 or 4 to complete a 3 to 7 membered carbocyclic ring which may be saturated or unsaturated; R⁸ is H, C₁₋₆alkyl, —CH₂OH, phenyl, phenyl(C₁₋₄alkyl) or CONR¹¹R¹²; R⁹ and R¹⁰ are each independently H, —CH₂OH, —C(O)NR¹¹R¹², C₁₋₆alkyl, phenyl (optionally substituted by C₁₋₄alkyl, halo or C₁₋₄alkoxy), or phenyl(C₁₋₄alkyl) (wherein the phenyl group is optionally substituted by C₁₋₄alkyl, halo or C₁₋₄alkoxy), or R⁹ and R¹⁰ together form a dioxolane; R¹¹ and R¹² which may be the same or different are H, C₁₋₄alkyl, R¹³ or S(O)ᵣR¹³, where r is 0, 1 or 2 and R¹³ is phenyl optionally substituted by C₁₋₄alkyl or phenyl C₁₋₄alkyl wherein the phenyl is optionally substituted by C₁₋₄alkyl; or

Y is the group, —C(O)NR¹¹R¹² wherein R¹¹ and R¹² are as previously defined except that R¹¹ and R¹² are not both H; or

Y is the group,

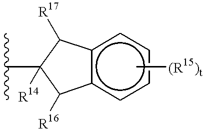

wherein R¹⁴ is H, CH₂OH, or C(O)NR¹¹R¹² wherein R¹¹ and R¹² are as previously defined; when present R¹⁵, which may be the same or different to any other R¹⁵, is OH, C₁₋₄alkyl, C₁₋₄alkoxy, halo or CF₃; t is 0, 1, 2, 3 or 4; and R¹⁶ and R¹⁷ are independently H or C₁₋₄alkyl; or

Y is the group

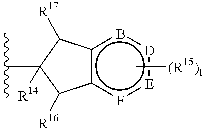

wherein one or two of B, D, E or F is a nitrogen, the others being carbon; and R¹⁴ to R¹⁷ and t are as previously defined; or

Y is an optionally substituted 5-7 membered heterocyclic ring, which may be saturated, unsaturated or aromatic and contains a nitrogen, oxygen or sulphur and optionally one, two or three further nitrogen atoms in the ring and which may be optionally benzofused and optionally substituted by:

C₁₋₆alkoxy; hydroxy; oxo; amino; mono or di-(C₁₋₄alkyl)amino; C₁₋₄alkanoylamino; or

C₁₋₆alkyl which may be substituted by one or more substituents, which may be the same or different, selected from the list: C₁₋₆alkoxy, C₁₋₆haloalkoxy, C₁₋₆alkylthio, halogen, C₃₋₇cycloalkyl, heterocyclyl or phenyl; or

C₃₋₇cycloalkyl, aryl or heterocyclyl, each of which may be substituted by one or more substituents, which may be the same or different, selected from the list: C₁₋₆alkyl, C₁₋₆alkoxy, C₁₋₆haloalkoxy, C₁₋₆alkylthio, halogen, C₃₋₇cycloalkyl, heterocyclyl or phenyl;

wherein when there is an oxo substitution on the heterocyclic ring, the ring only contains one or two nitrogen atoms and the oxo substitution is adjacent a nitrogen atom in the ring; or

Y is —NR¹⁸S(O)ᵤR¹⁹, wherein R¹⁸ is H or C₁₋₄alkyl; R¹⁹ is aryl, aryl C₁₋₄alkyl or heterocyclyl (preferably pyridyl); and u is 0, 1, 2 or 3.

Some of the compounds of formula I are disclosed in WO 91/10664 and WO 91/07386, but there is no teaching that they could be useful in the treatment of female sexual dysfunction. The remaining compounds of formula I are novel.

Therefore according to a second aspect, the invention provides a (novel) compound of formula (I), pharmaceutically acceptable salts, solvates, polymorphs or prodrugs thereof, wherein R¹, n and Y are as defined in the first aspect with the proviso that Y is not the group —C(O)NR¹¹R¹² and when R¹ is propyl or phenylethyl, R¹⁴ is not —CH₂OH.

According to a third aspect, the invention provides a (novel) compound of formula (I), pharmaceutically acceptable salts, solvates, polymorphs or prodrugs thereof, wherein R¹, n and Y are as defined in the first aspect with the proviso that Y is not the group —C(O)NR¹¹R¹² and R¹⁴ is not H or —CH₂OH.

In the above definition, unless otherwise indicated, alkyl groups having three or more carbon atoms may be straight or branched-chain. The term aryl as used herein means an aromatic hydrocarbon group such as phenyl or naphthyl which may optionally be substituted with, for example, one or more of OH, CN, CF₃, C₁-C₄ alkyl, C₁-C₄alkoxy, halo, carbamoyl, aminosulphonyl, amino, mono or di(C₁-C₄ alkyl)amino or (C₁-C₄ alkanoyl)amino groups.

Halo means fluoro, chloro, bromo or iodo.

In the above definition, unless otherwise indicated the term heterocyclyl means a 5 or 6 membered nitrogen, oxygen or sulphur containing heterocyclic group which, unless otherwise stated, may be saturated, unsaturated or aromatic and which may optionally include a further oxygen or one to three nitrogen atoms in the ring and which may optionally be benzofused or substituted with for example, one or more halo, C₁-C₄ alkyl, hydroxy, carbamoyl, benzyl, oxo, amino or mono or di-(C₁-C₄ alkyl)amino or (C₁-C₄ alkanoyl)amino groups. Particular examples of heterocycles include pyridyl, pyridonyl, pyrazinyl, pyrimidinyl, pyridazinyl, pyrrolyl, imidazolyl, pyrazolyl, triazolyl, tetrazolyl, furanyl, tetrahydrofuranyl, tetrahydropyranyl, dioxanyl, thienyl, oxazolyl, isoxazolyl, thiazolyl, oxadiazolyl, thiadiazolyl, indolyl, isoindolinyl, quinolyl, isoquinolyl, quinoxalinyl, quinazolinyl and benzimidazolyl, each being optionally substituted as previously defined.

Preferred R¹ substituents are C₁₋₆alkyl, C₁₋₆alkoxy, C₁₋₆alkoxy(C₁₋₃)alkyl, C₁₋₆alkoxyC₁₋₆alkoxyC₁₋₃alkyl or C₁₋₆alkyl substituted with aryl.

More preferred R¹ substituents are C₁₋₆alkyl, C₁₋₆alkoxy, C₁₋₆alkoxy(C₁₋₃)alkyl (preferably methoxyethyl) or C₁₋₆alkoxyC₁₋₆alkoxyC₁₋₃alkyl (preferably methoxyethoxymethyl).

Still more preferred R¹ substituents are C₁₋₄alkyl (preferably propyl) or C₁₋₆alkoxy(C₁₋₃)alkyl (preferably methoxyalkyl, more preferably methoxyethyl).

When Y is the group

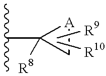

and the carbocyclic ring is fully saturated, then preferably one of R⁹ or R¹⁰ is —CH₂OH; —C(O)NR¹¹R¹²; C₁₋₆alkyl; phenyl optionally substituted by C₁₋₄alkyl; or phenyl(C₁₋₄alkyl) wherein the phenyl group is optionally substituted by C₁₋₄alkyl. More, preferably the carbocyclic ring is 5, 6 or 7 membered wherein one of R⁹ or R¹⁰, —C(O)NR¹¹R¹², with the other being C₁₋₆alkyl; phenyl optionally substituted by C₁₋₄alkyl; or phenyl(C₁₋₄alkyl) wherein the phenyl group is optionally substituted by C₁₋₄alkyl. More preferably, R⁹ and R¹⁰ are attached to adjacent carbon atoms in the ring. More preferably, R⁸ is CH₂OH.

When Y is the group —NR¹⁸S(O)ᵤR¹⁹, preferably R¹⁸ is H. More preferably, R¹⁹ is benzyl or phenyl. More preferably u is 2.

Preferably Y is an optionally substituted 5-7 membered heterocyclic ring. More preferably the ring is an optionally substituted aromatic ring, particularly pyridyl, pyrazinyl, pyrimidinyl, pyridazinyl, pyrazolyl, triazolyl, tetrazolyl, oxadiazolyl, thiazolyl, thiadiazolyl, oxazolyl, isoxazolyl, indolyl, isoindolinyl, quinolyl, isoquinolyl, pyridonyl, quinoxalinyl or quinazolinyl [especially oxadiazole (preferably 1,2,5- or 1,3,4-oxadiazole), pyridone (preferably 2-pyridone) or thiadiazole (preferably 1,3,4-thiadiazole) each of which may be substituted as defined in the first aspect. Preferably the heterocyclic ring is substituted by one or more C₁₋₆alkyl, phenyl or phenylC₁₋₄alkyl, more preferably by C₁₋₄alkyl or benzyl. Preferably Y is an N-substituted pyridone, preferably by benzyl or C₁₋₄alkyl.

Preferably Y is a lactam linked at the nitrogen.

Preferably Y is

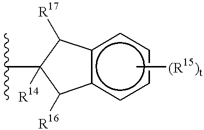

wherein R¹⁴ is preferably CH₂OH or C(O)NR¹¹R¹², especially C(O)NR¹¹R¹².

Preferably R¹⁶ and R¹⁷ are hydrogen. Preferably t is 0.

Preferred compounds are of formula Ie:

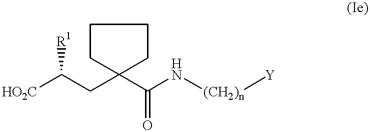

Particularly preferred compounds of the invention are:

2-[(1-{[(1-benzyl-6-oxo-1,6-dihydro-3-pyridinyl)amino]carbonyl}cyclopentyl)-methyl]-4-methoxybutanoic acid (Example 35);

2-{[l -({[3-(2-oxo-1-pyrrolidinyl)propyl]amino}carbonylcyclopentyl]-methyl}-4-phenylbutanoic acid (Example 40);

(+)-2-{[1-({[2-(hydroxymethyl)-2,3-dihydro-1H-inden-2-yl]amino}carbonyl)cyclopentyl]methyl}-4-phenylbutanoic acid (Example 44);

2-[(1-{[(5-methyl-1,3,4-thiadiazol-2-yl)amino]carbonyl}cyclopentyl)methyl]4-phenylbutanoic acid (Example 43);

cis-3-(2-methoxyethoxy)-2-[(1-{[(4-{[(phenylsulfonyl)amino]carbonyl}cyclohexyl)-amino]carbonyl}cyclopentyl)methyl]propanoic acid (Example 38);

(+)-2-{[1-({[2-(hydroxymethyl)-2,3-dihydro-1H-inden-2-yl]amino}carbonyl)cyclopentyl]-methyl}pentanoic acid (Example 31);

(2R)-2-((1-{[(5-ethyl-1,3,4-thiadiazol-2-yl)amino]carbonyl}cyclopentyl)-methyl]pentanoic acid or (−)-2-[(1-{[(5-ethyl-1,3,4-thiadiazol-2-yl)amino]carbonyl}cyclopentyl)-methyl]pentanoic acid (Example 29);

(2S)-2-[(l -{[(5-ethyl-1,3,4-thiadiazol-2-yl)amino]carbonyl}cyclopentyl)-methyl]pentanoic acid or (+)-2-[(1-{[(5-ethyl-1,3,4-thiadiazol-2-yl)amino]carbonyl}cyclopentyl)-methyl]pentanoic acid (Example 30);

2-({1-[(3-benzylanilino)carbonyl]cyclopentyl}methyl)pentanoic acid (Example 21);

2-[(1-{[(l -benzyl-6-oxo-1,6-dihydro-3-pyridinyl)amino]carbonyl}cyclopentyl)-methyl]pentanoic acid (Example 22);

2-{[1-({[(1R,3S,4R)-4-(aminocarbonyl)-3-butylcyclohexyl]amino}carbonyl)-cyclopentyl]methyl}pentanoic acid (Example 9);

trans-3-[1-({[2-(4-chlorophenyl)cyclopropyl]amino}carbonyl)cyclopentyl]-2-(methoxymethyl)propanoic acid (Example 46);

trans-3-[1-({[2-(4-methoxyphenyl)cyclopropyl]amino}carbonyl)cyclopentyl]-2-(methoxyethyl)propanoic acid (Example 47);

trans-3-[1-({[2-pentylcyclopropyl]aminocarbonyl)cyclopentyl]-2-(methoxyethyl)propanoic acid (Example 48);

3-[1-({[5-benzyl-[1,3,4]-thiadiazol-2-yl]amino}carbonyl)cyclopentyl]-2-(methoxyethyl)propanoic acid (Example 49);

3-[1-({[4-butylpyridin-2-yl]amino}carbonyl)cyclopentyl]-2-(methoxyethyl)propanoic acid (Example 50);

3-[1-({[4-phenylpyridin-2-yl]amino}carbonyl)cyclopentyl]-2-(methoxyethyl)propanoic acid (Example 51);

3-[1-({[1-hydroxymethyl-3-phenylcyclopentyl]amino}carbonyl)cyclopentyl]-2-(methoxyethyl)propanoic acid (Example 52);

2-{[1-({[2-(hydroxymethyl)-2,3-dihydro-1H-inden-2-yl]aminocarbonyl)-cyclopentyl]methyl}-4-methoxybutanoic acid (Example 53);

trans-3-[1-({[2-phenylcyclopropyl]amino}carbonyl)cyclopentyl]-2-(methoxyethyl)propanoic acid (Example 54);

(R)-2-{[1-({[2-(hydroxymethyl)-2,3-dihydro-1H-inden-2-yl]amino}carbonyl)-cyclopentyl]methyl}-4-methoxybutanoic acid (Example 55); and

(S)-2-{[1-({[2-(hydroxymethyl)-2,3-dihydro-1H-inden-2-yl]amino}carbonyl)-cyclopentyl]methyl}-4-methoxybutanoic acid (Example 56).

For the avoidance of doubt, unless otherwise indicated, the term substituted means substituted by one or more defined groups. In the case where groups may be selected from a number of alternatives groups, the selected groups may be the same or different.

For the avoidance of doubt, the term independently means that where more than one substituent is selected from a number of possible substituents, those substituents may be the same or different.

The pharmaceutically or veterinarily acceptable salts of the compounds of formula I which contain a basic center are, for example, non-toxic acid addition salts formed with inorganic acids such as hydrochloric, hydrobromic, hydroiodic, sulfuric and phosphoric acid, with carboxylic acids or with organo-sulfonic acids. Examples include the HCl, HBr, Hl, sulfate or bisulfate, nitrate, phosphate or hydrogen phosphate, acetate, benzoate, succinate, saccharate, fumarate, maleate, lactate, citrate, tartrate, gluconate, camsylate, methanesulfonate, ethanesulfonate, benzenesulfonate, p-toluenesulfonate and pamoate salts. Compounds of the invention can also provide pharmaceutically or veterinarily acceptable metal salts, in particular non-toxic alkali and alkaline earth metal salts, with bases. Examples include the sodium, potassium, aluminium, calcium, magnesium, zinc, diolamine, olamine, ethylenediamine, tromethamine, chloine, megulamine and diethanolamine salts. For reviews on suitable pharmaceutical salts see Berge et al, J. Pharm, Sci., 66, 1-19, 1977; P. L. Gould, International Journal of Pharmaceutics, 33 (1986), 201-217; and Bighley et al, Encyclopedia of Pharmaceutical Technology, Marcel Dekker Inc, New York 1996, Volume 13, page 453-497. A preferred salt is the sodium salt.

The pharmaceutically acceptable solvates of the compounds of the invention include the hydrates thereof.

Hereinafter, compounds, their pharmaceutically acceptable salts, their solvates and polymorphs, defined in any aspect of the invention (except intermediate compounds in chemical processes) are referred to as “compounds of the invention”.

The compounds of the invention may possess one or more chiral centers and so exist in a number of stereoisomeric forms. All stereoisomers and mixtures thereof are included in the scope of the present invention. Racemic compounds may either be separated using preparative HPLC and a column with a chiral stationary phase or resolved to yield individual enantiomers utilising methods known to those skilled in the art. In addition, chiral intermediate compounds may be resolved and used to prepare chiral compounds of the invention.

In cases where the compounds of the invention exist as the E and Z isomers, the invention includes individual isomers as well as mixtures thereof.

In cases where compounds of the invention exist as tautomeric isomers, the invention includes individual tautomers as well as mixtures thereof.

In cases where the compounds of the invention exist as optical isomers, the invention includes individual isomers as well as mixtures thereof.

In cases where the compounds of the invention exist as diastereoisomers, the invention includes individual diastereoisomers as well as mixtures thereof.

Separation of diastereoisomers or E and Z isomers may be achieved by conventional techniques, e.g. by fractional crystallisation, chromatography or H.P.L.C (see Examples 29 and 30 herein). An individual enantiomer of a compound of the invention or intermediate may be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active base, as appropriate. A preferred optically active base is pseudoephedrine (see Preparation 2 herein).

The compounds of the invention may exist in one or more tautomeric forms. All tautomers and mixtures thereof are included in the scope of the present invention. For example, a claim to 2-hydroxypyridinyl would also cover its tautomeric form, α-pyridonyl.

It will be appreciated by those skilled in the art that certain protected derivatives of compounds of the invention, which may be made prior to a final deprotection stage, may not possess pharmacological activity as such, but may, in certain instances, be administered orally or parenterally and thereafter metabolised in the body to form compounds of the invention which are pharmacologically active. Such derivatives may therefore be described as “prodrugs”. Further, certain compounds of the invention may act as prodrugs of other compounds of the invention.

All protected derivatives and prodrugs of compounds of the invention are included within the scope of the invention. Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference).

It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within the compounds of the invention.

Preferred prodrugs for compounds of the invention include: esters, carbonate esters, hemi-esters, phosphate esters, nitro esters, sulfate esters, sulfoxides, amides, carbamates, azo-compounds, phosphamides, glycosides, ethers, acetals and ketals.

Drug metabolism studies have shown that in vivo, compounds of formula I may form compounds of formula XXIII, which compounds also are inhibitors of NEP

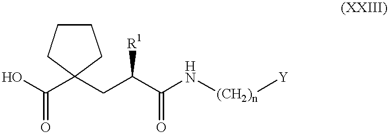

In particular, we have shown that (2R)-2-[(1-{[(5-ethyl-1,3,4-thiadiazol-2-yl)amino]carbonyl}cyclopentyl) methyl]pentanoic acid (Example 29) in vivo forms (2R)-1-(2-{[(5-ethyl-1,3,4-thiadiazol-2-yl)amino]carbonyl}pentyl)-cyclopentanecarboxylic acid.

The invention also includes all suitable isotopic variations of the compounds of the invention. An isotopic variation is defined as one in which at least one atom is replaced by an atom having the same atomic number but an atomic mass different from the atomic mass usually found in nature. Examples of isotopes that can be incorporated into compounds of the invention include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the invention, for example, those in which a radioactive isotope such as ³H or ¹⁴C is incorporated, are useful in drug and/or substrate tissue distribution studies. Tritiated, i.e. ³H, and carbon-14, i.e. ¹⁴C isotopes are particularly preferred for their ease of preparation and detectability. Further, substitution with isotopes such as deuterium, i.e. ²H, may afford certain therapeutic advantages resulting from greater metabolic stability, for example, increased in vivo half-life or reduced dosage requirements and hence may be preferred in some circumstances. Isotopic variations of the compounds of the invention can generally be prepared by conventional procedures such as by the methods or preparations described in the Examples and Preparations hereafter using appropriate isotopic variations of suitable reagents.

The compounds of the invention are inhibitors of the zinc-dependent, neutral endopeptidase EC.3.4.24.11., and it is proposed that the compounds of the invention will treat the disease states listed below. This enzyme is involved in the breakdown of several bioactive oligopeptides, cleaving peptide bonds on the amino side of hydrophobic amino acid residues. The peptides metabolised include atrial natriuretic peptides (ANP), bombesin, bradykinin, calcitonin gene-related peptide, endothelins, enkephalins, neurotensin, substance P and vasoactive intestinal peptide. Some of these peptides have potent vasodilatory and neurohormone functions, diuretic and natriuretic activity or mediate behaviour effects. Thus, the compounds of the invention, by inhibiting the neutral endopeptidase EC.3.4.24.11, can potentiate the biological effects of bioactive peptides.

Thus, in particular the compounds have utility in the treatment of a number of disorders, including hypertension, heart failure, angina, renal insufficiency, acute renal failure, cyclical oedema, Menieres disease, hyperaldosteroneism (primary and secondary) and hypercalciuria. In addition, because of their ability to potentiate the effects of ANF the compounds have utility in the treatment of glaucoma. As a further result of their ability to inhibit the neutral endopeptidase E.C.3.4.24.11 the compounds of the invention may have activity in other therapeutic areas including for example the treatment of menstrual disorders, preterm labour, pre-eclampsia, endometriosis, and reproductive disorders (especially male and female infertility, polycystic ovarian syndrome, implantation failure). Also the compounds of the invention should treat asthma, inflammation, leukemia, pain, epilepsy, affective disorders, dementia and geriatric confusion, obesity and gastrointestinal disorders (especially diarrhoea and irritable bowel syndrome), wound healing (especially diabetic and venous ulcers and pressure sores), septic shock, the modulation of gastric acid secretion, the treatment of hyperreninaemia, cystic fibrosis, restenosis, diabetic complications and athereosclerosis. In a preferred embodiment the compounds of the invention are useful in the treatment of female sexual dysfunction (FSD) preferably FSAD.

It is to be appreciated that all references herein to treatment include curative, palliative and prophylactic treatment.

We have found that the compounds of the invention inhibit the enzyme neutral endopeptidase. Therefore, according to a further aspect, the invention provides the use of a compound of the invention in the preparation of a medicament for the treatment or prophylaxis of a condition for which a beneficial therapeutic response can be obtained by the inhibition of neutral endopeptidase.

In accordance with the invention, FSD can be defined as the difficulty or inability of a woman to find satisfaction in sexual expression. FSD is a collective term for several diverse female sexual disorders (Leiblum, S. R. (1998). Definition and classification of female sexual disorders. Int. J. Impotence Res., 10, S104-S106; , Berman, J. R., Berman, L. & Goldstein, I. (1999). Female sexual dysfunction: Incidence, pathophysiology, evaluations and treatment options. Urology, 54, 385-391). The woman may have lack of desire, difficulty with arousal or orgasm, pain with intercourse or a combination of these problems. Several types of disease, medications, injuries or psychological problems can cause FSD. Treatments in development are targeted to treat specific subtypes of FSD, predominantly desire and arousal disorders.

The categories of FSD are best defined by contrasting them to the phases of normal female sexual response: desire, arousal and orgasm (Leiblum, S. R. (1998). Definition and classification of female sexual disorders, Int. J. Impotence Res., 10, S104-S106). Desire or libido is the drive for sexual expression. Its manifestations often include sexual thoughts either when in the company of an interested partner or when exposed to other erotic stimuli. Arousal is the vascular response to sexual stimulation, an important component of which is genital engorgement and includes increased vaginal lubrication, elongation of the vagina and increased genital sensation/sensitivity. Orgasm is the release of sexual tension that has culminated during arousal.

Hence, FSD occurs when a woman has an inadequate or unsatisfactory response in any of these phases, usually desire, arousal or orgasm. FSD categories include hypoactive sexual desire disorder, sexual arousal disorder, orgasmic disorders and sexual pain disorders. Although the compounds of the invention will improve the genital response to sexual stimulation (as in female sexual arousal disorder), in doing so it may also improve the associated pain, distress and discomfort associated with intercourse and so treat other female sexual disorders.

Thus, in accordance with a further aspect of the invention, there is provided the use of a compound of the invention in the preparation of a medicament for the treatment or prophylaxis of hypoactive sexual desire disorder, sexual arousal disorder, orgasmic disorder and sexual pain disorder, more preferably for the treatment or propylaxis of sexual arousal disorder, orgasmic disorder, and sexual pain disorder, and most preferably in the treatment or prophylaxis of sexual arousal disorder.

Hypoactive sexual desire disorder is present if a woman has no or little desire to be sexual, and has no or few sexual thoughts or fantasies. This type of FSD can be caused by low testosterone levels, due either to natural menopause or to surgical menopause. Other causes include illness, medications, fatigue, depression and anxiety.

Female sexual arousal disorder (FSAD) is characterised by inadequate genital response to sexual stimulation. The genitalia do not undergo the engorgement that characterises normal sexual arousal. The vaginal walls are poorly lubricated, so that intercourse is painful. Orgasms may be impeded. Arousal disorder can be caused by reduced oestrogen at menopause or after childbirth and during lactation, as well as by illnesses, with vascular components such as diabetes and atherosclerosis. Other causes result from treatment with diuretics, antihistamines, antidepressants eg SSRIs or antihypertensive agents.

Sexual pain disorders (includes dyspareunia and vaginismus) is characterised by pain resulting from penetration and may be caused by medications which reduce lubrication, endometriosis, pelvic inflammatory disease, inflammatory bowel disease or urinary tract problems.

The prevalence of FSD is difficult to gauge because the term covers several types of problem, some of which are difficult to measure, and because the interest in treating FSD is relatively recent. Many women's sexual problems are associated either directly with the female ageing process or with chronic illnesses such as diabetes and hypertension.

Because FSD consists of several subtypes that express symptoms in separate phases of the sexual response cycle, there is not a single therapy. Current treatment of FSD focuses principally on psychological or relationship issues. Treatment of FSD is gradually evolving as more clinical and basic science studies are dedicated to the investigation of this medical problem. Female sexual complaints are not all psychological in pathophysiology, especially for those individuals who may have a component of vasculogenic dysfunction (eg FSAD) contributing to the overall female sexual complaint. There are at present no drugs licensed for the treatment of FSD. Empirical drug therapy includes oestrogen administration (topically or as hormone replacement therapy), androgens or mood-altering drugs such as buspirone or trazodone. These treatment options are often unsatisfactory due to low efficacy or unacceptable side effects.

Since interest is relatively recent in treating FSD pharmacologically, therapy consists of the following: psychological counselling, over-the-counter sexual lubricants, and investigational candidates, including drugs approved for other conditions. These medications consist of hormonal agents, either testosterone or combinations of oestrogen and testosterone and more recently vascular drugs, that have proved effective in male erectile dysfunction. None of these agents has been demonstrated to be very effective in treating FSD.

As discussed, the compounds of the invention are particularly useful for the treatment of female sexual arousal disorder (FSAD).

The Diagnostic and Statistical Manual (DSM) IV of the American Psychiatric Association defines Female Sexual Arousal Disorder (FSAD) as being:

“a persistent or recurrent inability to attain or to maintain until completion of the sexual activity adequate lubrication-swelling response of sexual excitement. The disturbance must cause marked distress or interpersonal difficulty.”

The arousal response consists of vasocongestion in the pelvis, vaginal lubrication and expansion and swelling of the external genitalia. The disturbance causes marked distress and/or interpersonal difficulty.

FSAD is a highly prevalent sexual disorder affecting pre-, per- and post menopausal (±HRT) women. It is associated with concomitant disorders such as depression, cardiovascular diseases, diabetes and UG disorders.

The primary consequences of FSAD are lack of engorgement/swelling, lack of lubrication and lack of pleasurable genital sensation. The secondary consequences of FSAD are reduced sexual desire, pain during intercourse and difficulty in achieving an orgasm.

It has recently been hypothesised that there is a vascular basis for at least a proportion of patients with symptoms of FSAD (Goldstein et al., Int. J. Impot. Res., 10, S84-S90, 1998) with animal data supporting this view (Park et al., Int. J. Impot. Res., 9, 27-37, 1997).

Drug candidates for treating FSAD, which are under investigation for efficacy, are primarily erectile dysfunction therapies that promote circulation to the male genitalia. They consist of two types of formulation, oral or sublingual medications (Apomorphine, Phentolamine, phosphodiesterase type 5 (PDE5) inhibitors e.g. Sildenafil), and prostaglandin (PGE₁) that are injected or administered transurethrally in men, and topically to the genitalia in women.

The compounds of the invention are advantageous by providing a means for restoring a normal sexual arousal response—namely increased genital blood flow leading to vaginal, clitoral and labial engorgement. This will result in increased vaginal lubrication via plasma transudation, increased vaginal compliance and increased genital sensitivity. Hence, the compounds of the invention provide means to restore, or potentiate, the normal sexual arousal response.

Without being bound by theory, we believe that neuropeptides such as vasoactive intestinal peptide (VIP) are major neurotransmitter candidates in the control of the female sexual arousal response, especially in the control of genital blood flow. VIP and other neuropeptides are degraded/metabolised by NEP EC3.4.24.11. Thus, NEP inhibitors will potentiate the endogenous vasorelaxant effect of VIP released during arousal. This will lead to a treatment of FSAD, such as through enhanced genital blood flow and hence genital engorgement. We have shown that selective inhibitors of NEP EC 3.4.24.11 enhance pelvic nerve-stimulated and VIP-induced increases in vaginal and clitoral blood flow. In addition, selective NEP inhibitors enhance VIP and nerve-mediated relaxations of isolated vagina wall.

Thus the present invention is advantageous as it helps provide a means for restoring a normal sexual arousal response—namely increased genital blood flow leading to vaginal, clitoral and labial engorgement. This will result in increased vaginal lubrication via plasma transudation, increased vaginal compliance and increased vaginal sensitivity. Hence, the present invention provides a means to restore, or potentiate the normal sexual arousal response.

Background teachings on NEP have been presented by Victor A. McKusick et al on http://www3.ncbi.nlm.nih.gov/Omim/searchomim.htm. The following information concerning NEP has been extracted from that source:

“Common acute lymphocytic leukemia antigen is an important cell surface marker in the diagnosis of human acute lymphocytic leukemia (ALL). It is present on leukemic cells of pre-B phenotype, which represent 85% of cases of ALL, CALLA is not restricted to leukemic cells, however, and is found on a variety of normal tissues. CALLA is a glycoprotein that is particularly abundant in kidney, where it is present on the brush border of proximal tubules and on glomerular epithelium. Letarte et al. (1988) cloned a cDNA coding for CALLA and showed that the amino acid sequence deduced from the cDNA sequence is identical to that of human membrane-associated neutral endopeptidase (NEP; EC 3.4.24.11 ), also known as enkephalinase. NEP cleaves peptides at the amino side of hydrophobic residues and inactivates several peptide hormones including glucagon, enkephalins, substance P, neurotensin, oxytocin, and bradykinin. By cDNA transfection analysis, Shipp et al. (1989) confirmed that CALLA is a functional neutral endopeptidase of the type that has previously been called enkephalinase. Barker et al. (1989) demonstrated that the CALLA gene, which encodes a 100-kD type 11 transmembrane glycoprotein, exists in a single copy of greater than 45 kb which is not rearranged in malignancies expressing cell surface CALLA. The gene was located to human chromosome 3 by study of somatic cell hybrids and in situ hybridization regionalized the location to 3q21-q27. Tran-Paterson et al. (1989) also assigned the gene to chromosome 3 by Southern blot analysis of DNA from human-rodent somatic cell hybrids. D'Adamio et al. (1989) demonstrated that the CALLA gene spans more than 80 kb and is composed of 24 exons.”

1 Barker, P. E.; Shipp, M. A.; D'Adamio, L.; Masteller, E. L.; Reinherz, E. L. The common acute lymphoblastic leukemia antigen gene maps to chromosomal region 3(q21-q27). J. Immun. 142: 283-287, 1989.

2 D'Adamio, L.; Shipp, M. A.; Masteller, E. L.; Reinherz, E. L. : Organization of the gene encoding common acute lymphoblastic leukemia antigen (neutral endopeptidase 24.11): multiple miniexons and separate 5-prime untranslated regions. Proc. Nat. Acad. Sci. 86: 7103-7107, 1989.

3 Letarte, M.; Vera, S.; Tran, R.; Addis, J. B. L.; Onizuka, R. J.; Quackenbush, E. J.; Jongeneel, C. V.; McInnes, R. R. : Common acute lymphocytic leukemia antigen is identical to neutral endopeptidase. J. Exp. Med. 168: 1247-1253, 1988.

4 Shipp, M. A.; Vijayaraghavan, J.; Schmidt, E. V.; Masteller, E. L.; D'Adamio, L.; Hersh, L. B.; Reinherz, E. L. : Common acute lymphoblastic leukemia antigen (CALLA) is active neutral endopeptidase 24.11 (‘enkephalinase’): direct evidence by cDNA transfection analysis. Proc. Nat. Acad. Sci. 86: 297-301, 1989.

5 Tran-Paterson, R.; Willard, H. F.; Letarte, M.: The common acute lymphoblastic leukemia antigen (neutral endopeptidase—3.4.24.11) gene is located on human chromosome 3. Cancer Genet. Cytogenet. 42: 129-134, 1989.

“The (female) genital organs consist of an internal and external group. The internal organs are situated within the pelvis and consist of ovaries, the uterine tubes, uterus and the vagina. The external organs are superficial to the urogenital diaphragm and below the pelvic arch. They comprise the mons pubis, the labia majora and minora pudendi, the clitoris, the vestibule, the bulb of the vestibule, and the greater vestibular glands” (Gray's Anatomy, C. D. Clemente, ₁₃th American Edition).

The compounds of the invention find application in the following sub-populations of patients with FSD: the young, the elderly, pre-menopausal, peri-menopausal, post-menopausal women with or without hormone replacement therapy.

The compounds of the invention find application in patients with FSD arising from:

i) Vasculogenic etiologies eg cardiovascular or atherosclerotic diseases, hypercholesterolemia, cigarette smoking, diabetes, hypertension, radiation and perineal trauma, traumatic injury to the iliohypogastric pudendal vacular system.

ii) Neurogenic etiologies such as spinal cord injuries or diseases of the central nervous system including multiple sclerosis, diabetes, Parkinsonism, cerebrovascular accidents, peripheral neuropathies, trauma or radical pelvic surgery.

iii) Hormonal/endocrine etiologies such as dysfunction of the hypothalamic/pituitary/gonadal axis, or dysfunction of the ovaries, dysfunction of the pancreas, surgical or medical castration, androgen deficiency, high circulating levels of prolactin eg hyperprolactinemia, natural menopause, premature ovarian failure, hyper and hypothyroidism.

iv) Psychogenic etiologies such as depression, obsessive compulsive disorder, anxiety disorder, postnatal depression/“Baby Blues”, emotional and relational issues, performance anxiety, marital discord, dysfunctional attitudes, sexual phobias, religious inhibition or a traumatic past experiences.

v) Drug-induced sexual dysfunction resulting from therapy with selective serotonin reuptake inhibitors (SSRis) and other antidepressant therapies (tricyclics and major tranquillizers), anti-hypertensive therapies, sympatholytic drugs, chronic oral contraceptive pill therapy.

Compounds of the invention may be prepared, in known manner in a variety of ways. In the following reaction schemes and hereafter, unless otherwise stated R¹, n and Y are as defined in the first aspect. These processes form further aspects of the invention.

Throughout the specification, general formulae are designated by Roman numerals II, III, IV etc. Subsets of these general formulae are defined as Ia, Ib, Ic etc., . . . IVa, IVb, IVc etc.

Compounds of general formula I may be prepared according to reaction scheme 1, by reacting a compound of formula II (where Prot is a suitable protecting group) with a primary amine of formula III to give a compound of formula IV. Deprotection gives compounds of formula I.

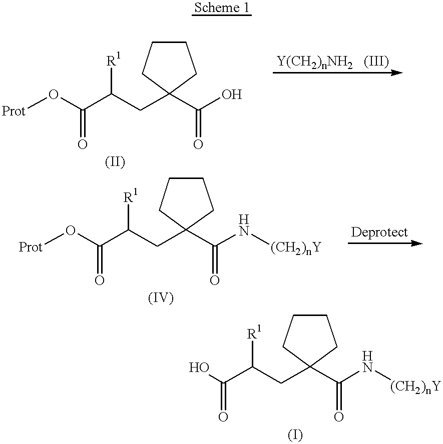

The acid/amine coupling step can be carried out by reacting compounds of formula II with compounds of formula III (or its amine salt) in the presence of a coupling agent, optionally a catalyst, and an excess of an acid acceptor, in a suitable solvent. Typically, treatment of a mixture of compounds of formula II and compounds of formula III with a coupling agent (for example dicyclohexylcarbodiimide (DCC), 1-(3-dimethylaminopropyl)-3-ethylcarbodiimide (WSCDI), benzotriazol-1-yl diethyl phosphate, phosphorus oxychloride, titanium tetrachloride, sulfuryl chloride fluoride, Lawesson's reagent, PPACA, PYBOP or Mukaiyama's reagent) optionally in the presence of a tertiary amine base (for example triethylamine, Hunig's base, pyridine or NMM) for up to 24 hours at temperatures between −78 and 100° C. Preferred reaction conditions comprise reacting compounds of formula II (1-1.5 equivalents) with compounds of formula III (or their salts 1-1.5 equivalents), in the presence of 1-(3-dimethylaminopropyl)-3-ethylcarbodiimide hydrochloride (WSCDI) or N,N′-dicyclohexylcarbodiimide (DCC) (1.1-1.3 equivalents), 1-hydroxybenzotrazole hydrate (HOBT) or dimethylaminopyridine (DMAP) (1.05-1.2 equivalents), N-methyl morpholine (NMM) or triethyamine (2.3-3 equivalents) in dimethylformamide or dichloromethane at between room temperature and 90° C. for 16-18 hours.

Alternatively, the acid/amine coupling step may proceed via an activated intermediate (such as an acyl imidazolide, mixed anhydride or acid chloride) in the presence of an excess of acid acceptor in a suitable solvent. Typical reaction conditions comprise treatment of compounds of formula II with an activating agent (for example N,N′-carbonyldiimidazole, N,N′-carbonylbis(3-methylimidazolium) triflate, thionyl chloride or oxalyl chloride) optionally in the presence of a tertiary amine base (for example triethylamine, Hunig's base, pyridine or NMM) for up to 24 hours followed by reaction with compounds of formula III (or its salt), optionally in the presence of a catalyst (for example 4-dimethylaminopyridine) or an additive (for example hydroxybenzotriazole) in a suitable solvent (for example dichloromethane, THF, ethyl acetate, acetonitrile, DMF or toluene) optionally in the presence of an additional amine base at temperatures between −78° C. and 150° C. for up to 48 hours.

Preferred reaction conditions comprise reacting the acid chloride of compounds of formula II (1-1.1 equivalents) with compounds of formula III (or their salts, 1 to 1.5 equivalents) in the presence of triethyamine or N-methyl morpholine (1.4-10 equivalents) in dichloromethane solvent at room temperature for 24 hours. Alternatively, compounds of formula II can be converted to the acid chloride in situ by treatment with oxalyl chloride in dichloromethane in the presence of a catalytic amount of dimethylformamide for 2 hours at room temperature or by treatment of compounds of formula II with thionyl chloride in a mixture of dichloromethane and pyridine at −10° C. for 3 hours followed by addition of triethylamine, 4-dimethylaminopyridine and the compound of formula III and allowing the mixture to react for 48 hours at 20° C.

Compounds of formula I may be prepared from compounds of formula IV by deprotection. Methods for deprotection of an acid group depend on the protecting group. For examples of protection/deprotection methodology see “Protective groups in Organic synthesis”, T. W. Greene and P. G. M. Wutz.

For example, when Prot is a tert-butyl, deprotection conditions comprise reacting IV with trifluoroacetic acid/dichloromethane (1:1-1.5 by volume), at room temperature for 2-18 hours, optionally in the presence of a carbocation scavenger, e.g. anisole (10 equivalents). When Y contains a hydroxy group, base hydrolysis of the intermediate trifluoroacetic acid ester may be necessary. Alternative methodology for deprotection when Prot is tert-butyl comprises treating IV with hydrochloric acid in dichloromethane at room temperature for 3 hours. For the avoidance of doubt, Prot as tert-butyl is given by way of Example and is not intended to be limited to tert-Butyl.

Alternatively, when Prot is tert-butyl deprotection may be achieved by treating compounds of formula IV with a strong acid (for example gaseous or concentrated hydrochloric acid, hydrobromic acid, phosphoric acid, nitric acid or sulfuric acid, trifluoroacetic acid, chloroacetic acid, para-toluenesulfonic acid, trifluoromethanesulfonic acid or glacial acetic acid) in quantities ranging from catalytic to an excess, optionally in a suitable solvent (for example toluene, dichloromethane, diethyl ether, ethanol, THF or hexane) and optionally in the presence of water at temperatures between 20° C. and 150° C. for up to 48 hours.

Preferred deprotection conditions when Prot is tert-butyl are treatment of compounds of formula IV with a ten-fold excess of trifluoroacetic acid in dichloromethane at room temperature for 24 hours.

When Prot is benzyl, deprotection conditions comprise reacting IV with palladium on charcoal (5-10%) in aqueous ethanol (40-95%) at 15-60 psi at room temperature for 2 hrs to 3 days.

These processes form further aspects of the invention.

Compounds of formula IV are novel and form a further aspect of the invention.

Compounds of formula la, i.e. compounds of general formula I where Y is —NHSO₂R¹⁹, may be prepared according to reaction scheme 2. Compounds of formula V are first prepared by reacting compounds of formula II with compounds of formula VI where Prot² is a suitable amine protecting group. Preferred reaction conditions are analogous to those described for the acid/amine coupling step in Scheme 1 above. Selective amine deprotection of compounds of formula V gives compounds of formula VII. Compounds of formula VII are reacted with R¹⁹SO₂Cl in the presence of an acid acceptor in a suitable solvent to form compounds of formula IVa. Deprotection of compounds of formula IVa under analogous conditions to those described for the deprotection step of Scheme 1 gives compounds of formula Ia.

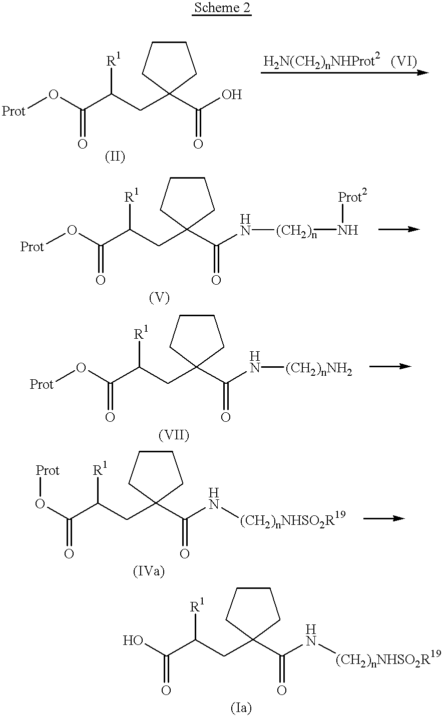

Methods for deprotection of an amine group depend on the protecting group. For examples of protection/deprotection methodology see “Protective groups in Organic Synthesis”, T. W. Greene and P. G. M. Wutz. For example, when Prot² is benzoyloxycarbonyl, deprotection conditions comprise reacting V with palladium on charcoal (10%) in ethanol at room temperature for 18 hours.

Preferred methods for preparation of the compounds of formula IVa comprise reaction of VII with R¹⁹SO₂Cl (1 equivalent) in the presence of triethyamine (1.5-2.5 equivalents) in dichloromethane at room temperature for 2 to 3 days.

Compounds of formula Ib, i.e. compounds of formula I where n is 0 and Y is

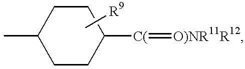

may be prepared according to reaction scheme 3. Compounds of formula II are reacted with compounds of formula IlIa under analogous conditions to acid/amine coupling conditions of Scheme 1 to give compounds of formula IX, where Prot³ is a protecting group which can be selectively removed in the presence of protecting group Prot. A preferred protecting group Prot³ is a base labile ester group. Consequently, treatment of compound of formula IX under basic conditions gives compounds of formula X. Compounds of formula X are reacted with compounds of formula NHR¹¹R¹² under analogous conditions to acid/amine coupling conditions of Scheme 1 to form compounds of formula IVb. Deprotection of compounds of formula IVb under analogous conditions to the deprotection step in Scheme 1 gives compounds of formula Ib.

Preferred conditions for removal of protecting group Prot³ from IVb comprise treatment of IVb with sodium hydroxide (1N) in methanol at room temperature for 22 hours.

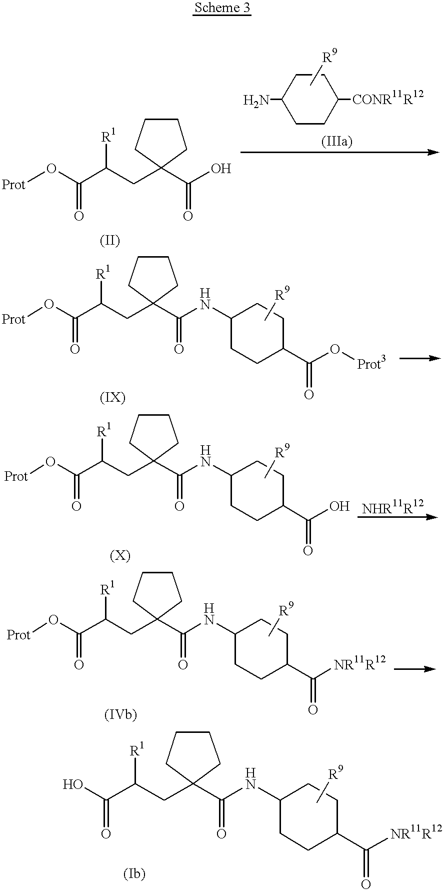

Compounds of formula IIIb, i.e. compounds of general formula III where n is 2 and Y is 2-oxopiperidino, may be prepared according to reaction scheme 4.

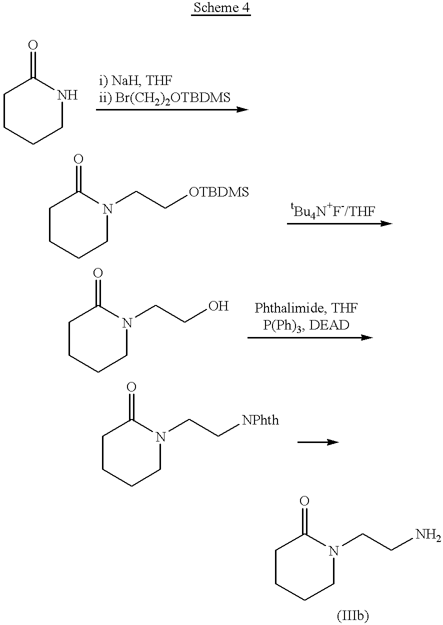

Compounds of formula IIIc where r is 1 or 2, may be prepared according to reaction scheme 5. Compounds of formula XII are protected at the amine moiety with a suitable protecting group Pro⁴ to form compounds of formula XII. A preferred protecting group is tert-butyloxycarbonyl. Compounds of formula XII are reacted under typical acid/amine coupling conditions with NHR¹¹R¹² to form compounds of formula XIV, which on deprotection form compounds of formula IIIc.

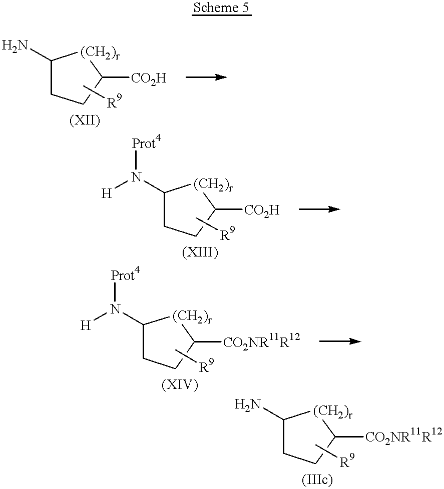

Typical reaction conditions for introducing the tert-butyloxycarbonyl protecting group comprise treating compounds of formula XII with (tert-butyloxycarbonyl)₂O in dioxan and 2N sodium hydroxide at room temperature for 18 hrs.

Typical acid/amine coupling conditions comprise treating compounds of formula XIII and NHR¹¹R¹² with benzotriazol-1-yloxytris(pyrrolidino)phosphonium hexafluorophosphate (PYBOP), 1-hydroxybenzotrazole hydrate (HOBT), Hunigs base, an amine (eg triethylamine), in dimethylformamide at room temperature for 2 hrs. Alternatively, compounds of formula XIII and NHR¹¹R¹² may be treated with 1-(3-dimethylaminopropyl)-3-ethylcarbodiimide hydrochloride, HOBT, N-methyl morpholine (NMM), in dimethylformamide at room temperature for 18 hrs.

Typical reaction conditions for deprotection when Prot⁴ is tert-butyloxycarbonyl comprise reacting XIV with hydrochloric acid or trifluoroacetic acid in dichloromethane at room temperature for 2 to 4 hrs

Compounds of formula IIId may be prepared according to reaction scheme 6. The protecting group is preferably tert-butyloxycarbonyl, which may be removed under standard conditions, as previously described.

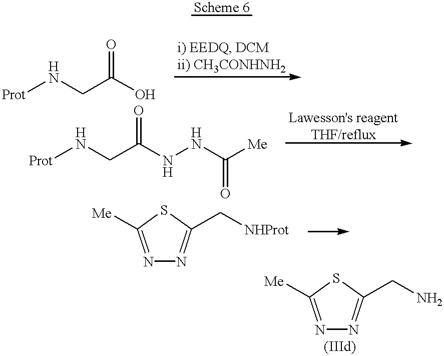

Compounds of formula IIIe may be prepared according to reaction scheme 7 using standard acid/amine coupling reactions, as previously described. The protecting group is preferably benzyloxycarbonyl which may be removed under standard conditions, typically palladium on charcoal (5-10%) in ethanol at room temperature and 50 psi for 4 hrs.

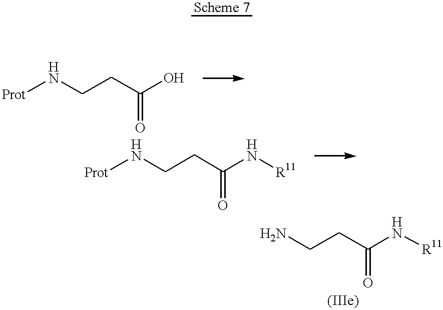

Compounds of formula IIIf may be prepared according to reaction scheme 8.

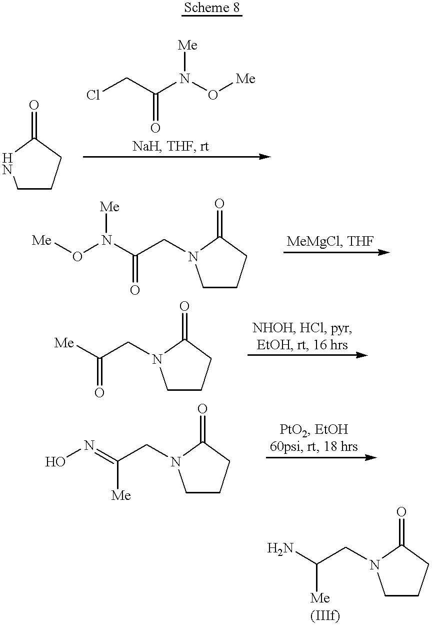

Compounds of formula IIIg may be prepared in two steps according to reaction scheme 9. As a first step, compounds of formula XV may be prepared from compounds of formula XVI using standard acid/amine coupling methodology analogous to the acid/amine coupling conditions described for reaction scheme 1. Prot⁵ represents a suitable leaving group, preferably tertbutyloxycarbonyl. The second step comprises removal of Prot⁵. When Prot⁵ is tertbutyloxycarbonyl then preferred reaction conditions comprise treatment with hydrochloric acid in diethyl ether/ethyl acetate at room temperature for 18 hrs.

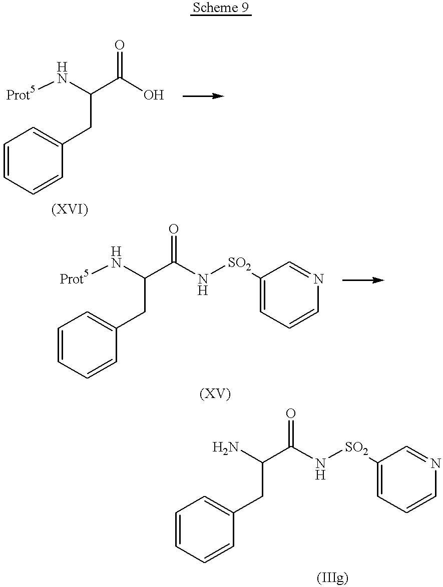

Compounds of formula IIIh may be prepared in three steps according to reaction scheme 10.

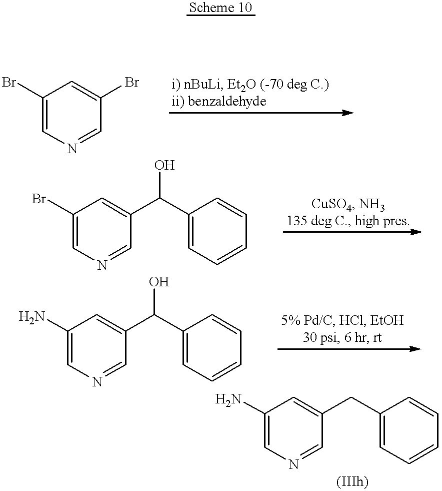

Compounds of formula IIIj may be prepared by reduction of a nitro group according to reaction scheme 11.

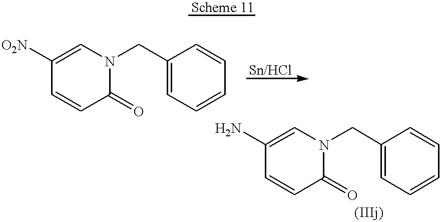

Further methods for preparing compounds of formula III are given in Scheme 12 below, where R^{a} is C₁₋₆alkyl or alkoxy.

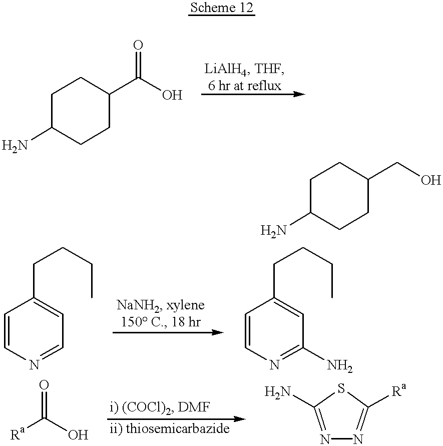

Compounds of general formula I possess a chiral center at the carbon attached to R¹. Individual enantiomers of general formula I may be obtained by a variety of methods known to the skilled chemists, such as from a corresponding optically pure intermediate or via resolution. A preferred method of resolution is via the (+)-pseudoephedrine salt (see Preparation 2 herein). Alternatively, chiral compounds of formula I may be prepared from chiral compounds of formula II as discussed below.

A number of compounds of formula II are known in the art (see EP274234-B1 and WO9113054). Other compounds of formula II can be prepared in analogous fashion. Chiral compounds of formula Ha may be prepared from compounds of formula XVI as shown in reaction scheme 13.

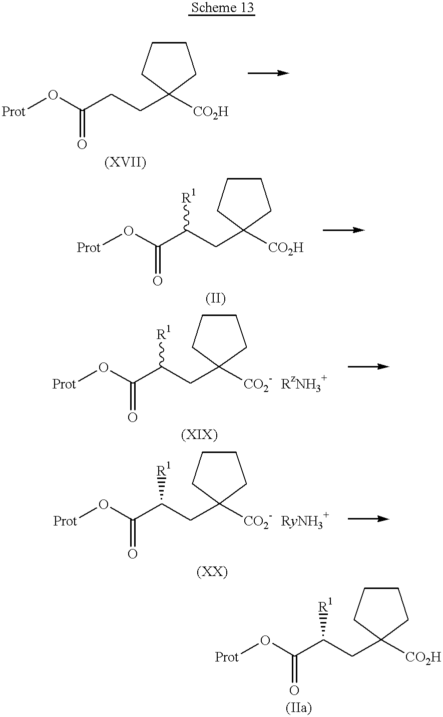

Compounds of formula II may be prepared by treatment of compounds of formula XVII with R¹-X^{z} (where X^{z} is halogen) under strongly basic conditions optionally with an additive in an aprotic solvent.

Typical reaction conditions comprise firstly treating compounds of formula XVII with at least a two-fold excess of a strong base (for example lithium diisopropylamide, lithium, sodium or potassium hexamethyldisilazide, an alkyllithium, alkylmagnesium or phosphazene base) in a non-protic solvent (for example THF, diethyl ether, hexane, heptane or ethylbenzene or a mixture of these solvents), optionally with an additive (for example TMEDA, DMPU or HMPA) at temperatures between −78° C. and room temperature, and then adding R¹-X^{z} (for example allyl bromide, propyl bromide or methyl iodide) at −78° C. and stirring overnight whilst warming to room temperature. A suitable work-up gives the compounds of formula II.

Preferred reaction conditions for preparing compounds of formula II where R¹ is allyl, are treatment of compounds of formula XVII (1 molar equivalent) with lithium diisopropylamide (2.3 equivalents) in a mixture of THF, n-heptane and ethylbenzene at −10° C. for 4 hours. Allyl bromide (1.2 equivalents) is then added at −10° C., the reaction mixture stirred at −10° C. for 2 hours and then warmed to 20° C. over 4 hours and stirred for a further 15 hours at 20° C.

Resolution to compounds of formula IIb may be performed directly from compounds of formula II, however a preferred process forms an amine salt of formula XIX (i.e. R^{z}—NH₃⁺) followed by recrystallisation to effect purification.

Typically salts are the triethylamine, isopropylamine, triethanolamine or cyclohexylamine salts. Typical reaction conditions comprise reacting II with the required amine in a suitable solvent (for example hexane, heptane or toluene) at elevated temperatures with cooling to induce crystallisation over 24 hours, followed by recrystallisation from the same or a different solvent, optionally at elevated temperatures.

A preferred salt is the cyclohexylamine salt. Preferred reaction conditions comprise treatment compounds of formula II (1 molar equivalent) with cyclohexylamine (1 equivalent) at 20° C. in heptane followed by recrystallisation from ethyl acetate at 70° C. before cooling to 50° C. over 2 hours to induce crystallisation. The mixture is then cooled to 20° C. over 2 hours and stirred for 0.5 hours.

Compounds of formula XX (where R^{y}NH₃⁺ is a chrial cation) may be prepared from compounds of formula XIX by acidification in a suitable solvent system, optionally including water using a strong acid followed by classical resolution of the resulting carboxylic acid.

Typical reaction conditions comprise treatment of compounds of formula XX in a biphasic system of water and an immiscible organic solvent (for example heptane, toluene, ethyl acetate, diethyl ether or dichloromethane) with a strong acid (for example hydrochloric acid, sulfuric acid, para-toluenesulfonic acid, trifluoroacetic acid or phosphoric acid) at temperatures between 0° C. and 100° C. to give the free carboxylic acid, followed by treatment with a non-racemic chiral amine base (for example α-methylbenzylamine, pseudoephedrine, ephedrine, norephedrine, a cinchona alkaloid, amino acid esters, amino alcohols such as 2-pyrrolidinemethanol or quinuclidin-3-ol) optionally in a solvent (for example an ester, an alkane, an aromatic hydrocarbon, a haloalkane, an ether or an alcohol) at temperatures between 0° C. and 150° C. to give the crude salt. This salt is then recrystallised one or more times from the same or a different solvent at temperatures between 0° C. and 150° C., to give the chiral salt.

A preferred salt is the pseudoephedrine salt. Preferred reaction conditions comprise treatment of a suspension of a compound of formula XX in a water/n-heptane mixture with dilute hydrochloric acid at room temperature until the pH of the aqueous phase is pH 3 to give the free acid, followed by treatment of the resulting carboxylic acid with (1S, 2S)-(+)-pseudoephedrine (1 equivalent) in n-heptane at 80° C. followed by cooling to 45° C. over 2 hours to induce crystallisation then cooling to 20° C. over 2 hours and stirring for 4 hours. Recrystallisation from n-heptane was then carried out at 80° C. followed by cooling to 60° C. over 2 hours to induce crystallisation then cooling to 20° C. over 2 hours and stirring for 1.5 hours.

Compounds of formula IIa may be prepared from compounds of formula XX by acidification in a suitable solvent system, optionally including water using a strong acid.

Typical reaction conditions comprise treatment of compounds of formula XX in a biphasic system of water and an immiscible organic solvent (for example heptane, toluene, ethyl acetate, diethyl ether or dichloromethane) and a strong acid (for example hydrochloric acid, sulfuric acid, para-toluenesulfonic acid, trifluoroacetic acid or phosphoric acid) at temperatures between 0° C. and 100° C. to give compounds of formula IIa.

Preferred reaction conditions comprise treatment of a suspension of a compound of formula XX in a water/n-heptane mixture with dilute hydrochloric acid at room temperature until the pH of the aqueous phase is pH 3 to give the free acid.

Compounds of formula IIa may be prepared by hydrogenation of the corresponding compound where R¹ is unsaturated. Typical reaction conditions comprise stirring under an atmosphere of hydrogen in a suitable solvent in the presence of a catalyst (for example palladium, platinum, nickel, iridium, rhodium or ruthenium optionally adsorbed on a suitable support such as carbon, alumina, barium sulfate, calcium carbonate or as a salt such as palladium hydroxide, or mixtures of salts such as H₂PtCl₆ and SnCl₂•2H₂O or as a complex such as Wilkinson's catalyst, Crabtree's catalyst, Co₂(CO)₈, RhH(PPh₃)₄, or [Co(CN)₅]³⁻) at temperatures between room temperature and 150° C. and hydrogen pressures between 30 and 150 psi.

In a preferred process, compounds of formula IIa where R¹ is propyl, may be prepared by hydrogenation of the corresponding allyl compound. Preferred reaction conditions comprise stirring an ethanol solution of the unsaturated carboxylic acid under a hydrogen atmosphere with 9% w/w of 5% palladium on carbon at room temperature for 24 hours.

Compounds of formula XVIIa, i.e. compounds of formula XVII where Prot is tert-butyl, may be prepared in two steps from commercially available compounds of formula XXI according to reaction scheme 14.

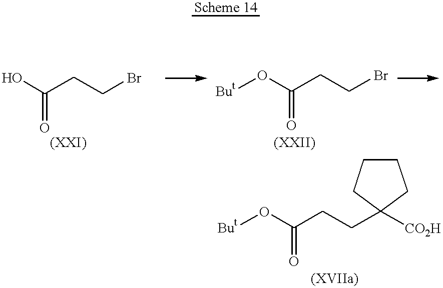

Compounds of formula XXII may be prepared from compounds of formula XXI by treating XXI with a source of tert-butyl cation or tert-butoxide using a suitable catalyst and/or dehydrating agent in a suitable anhydrous solvent optionally at elevated temperature, or by activation of the carboxylic acid followed by reaction with tert-butanol.

Typical reaction conditions comprise treating compounds of formula XXI with catalytic quantities of an acid (for example phosphoric acid, hydrochloric acid, sulfuric acid, nitric acid, para-toluenesulfonic acid or trifluoroacetic acid) in the presence of isobutylene, tert-butanol, tert-butyl halides or tert-butyl ether in a suitable solvent (for example dichloromethane, THF or toluene) between −20 and 150° C. for up to 48 hours.

Alternative reaction conditions comprise treating compounds of formula XXI with a combination of a tertiary amine base (for example triethylamine, Hunig's base, pyridine or NMM) and a dehydrating agent (for example dicyclohexylcarbodiimide, an alkyl chloroformate, phenyl dichlorophosphate, 2-chloro-1,3,5-trinitrobenzene, di-2-pyridyl carbonate, 1,1′-carbonyl diimidazole, (trimethylsilyl)ethoxyactylene, N,N′-carbonylbis(3-methylimidazolium) triflate or diethyl azodicarboxylate) and triphenyl phosphine followed by addition of tert-butanol, optionally with a catalyst such as 4-dimethylaminopyridine in a suitable solvent (for example dichloromethane, THF or toluene) between −20 and 150° C. for up to 48 hours.

Still further reaction conditions comprise converting compounds of formula XXI to the acid chloride using thionyl chloride, oxalyl chloride or Ghosez's reagent optionally in the presence of a tertiary amine base (for example triethylamine, Hunig's base, pyridine or NMM) followed by treatment with tert-butanol, optionally in the presence of a catalys such as 4-dimethylaminopyridine in a suitable solvent (for example dichloromethane, THF or toluene) between −20 and 150° C. for up to 48 hours.

Preferred reaction conditions comprise treating compounds of formula XXI with isobutylene (5 equivalents), concentrated sulfuric acid (0.15 equivalents) and tert-butanol (0.16 equivalents) in dichloromethane stirring at −10 to 25° C. for 24 hours.

Compounds of formula XVIIa may be prepared from compounds of formula XXII by treatment with cyclopentane carboxylic acid under strongly basic conditions in an aprotic solvent, optionally in the presence of an additive.

Typical reaction conditions comprise treating cyclopentane carboxylic acid with at least a two-fold excess of a strong base (for example lithium diisopropylamide, lithium, sodium or potassium hexamethyldisilazide, an alkyllithium, alkylmagnesium or phosphazene base) in a non-protic solvent (for example THF, diethyl ether, hexane, heptane or ethylbenzene or a mixture of these), optionally in the presence of an additive (for example TMEDA, DMPU or HMPA) at temperatures ranging from −78° C. to 50° C. temperature for up to 24 hours, followed by the addition of the compound of formula XXII and reaction at −20° C. for up to 24 hours and suitable workup.

Preferred reaction conditions comprise treating of cyclopentane carboxylic acid with lithium diisopropylamide (2.15 equivalents) in a mixture of THF, n-heptane and ethylbenzene at −15° C. for 3 hours followed by treatment with tert-butyl-3-bromopropionate (1.06 equivalents) in THF at −15° C. for 15 hours then warming to room temperature.

Other compounds of formula II are either available from commercial sources, known in the prior art, or can be prepared from compounds known in the prior art by using methods known in the prior art or by using methods described herein (see Examples and Preparations Sections).

A pharmaceutically acceptable salt of a compound of the formula (I) may be readily prepared by mixing together solutions of a compound of the formula (I) and the desired acid or base, as appropriate. The salt may precipitate from solution and be collected by filtration or may be recovered by evaporation of the solvent.

The compounds of the invention may also be combined with one or more of the following for the treatment of FSD:

1) One or more naturally occurring or synthetic prostaglandins or esters thereof. Suitable prostaglandins for use herein include compounds such as alprostadil, prostaglandin E₁,prostaglandin E₀, 13,14-dihydroprosta glandin E₁, prostaglandin E₂, eprostinol, natural synthetic and semi-synthetic prostaglandins and derivatives thereof including those described in WO-00033825 and/or U.S. Pat. No. 6,037,346 issued on Mar. 14, 2000 all incorporated herein by reference, PGE₀, PGE₁, PGA₁, PGB₁, PGF₁α, 19-hydroxy PGA₁, 19-hydroxy-PGB₁, PGE₂, PGB₂, 19-hydroxy-PGA₂, 19-hydroxy-PGB₂, PGE₃α, carboprost tromethamine dinoprost, tromethamine, dinoprostone, lipo prost, gemeprost, metenoprost, sulprostune, tiaprost and moxisylate.

2) One or more α-adrenergic receptor antagonist compounds also known as α-adrenoceptors or α-receptors or α-blockers. Suitable compounds for use herein include: the α-adrenergic receptor blockerss as described in PCT application WO99/30697 published on Jun. 14, 1998, the disclosures of which relating to α-adrenergic receptors are incorporated herein by reference and include, selective α₁-adrenoceptor or α₂-adrenoceptor blockers and non-selective adrenoceptor blockers, suitable α₁-adrenoceptor blockers include: phentolamine, phentolamine mesylate, trazodone, alfuzosin, indoramin, naftopidil, tamsulosin, dapiprazole, phenoxybenzamine, idazoxan, efaraxan, yohimbine, rauwolfa alkaloids, Recordati 15/2739, SNAP 1069, SNAP 5089, RS17053, SL 89.0591, doxazosin, terazosin, abanoquil and prazosin; α₂-blocker blockers from U.S. Pat. No. 6,037,346 [Mar. 14, 2000] dibenarnine, tolazoline, trimazosin and dibenarnine; α-adrenergic receptors as described in U.S. Pat. Nos.: 4,188,390; 4,026,894; 3,511,836; 4,315,007; 3,527,761; 3,997,666; 2,503,059; 4,703,063; 3,381,009; 4,252,721 and 2,599,000 each of which is incorporated herein by reference; α₂-Adrenoceptor blockers include: clonidine, papaverine, papaverine hydrochloride, optionally in the presence of a cariotonic agent such as pirxamine.

3) One or more NO-donor (NO-agonist) compounds. Suitable NO-donor compounds for use herein include organic nitrates, such as mono- di or tri-nitrates or organic nitrate esters including glyceryl brinitrate (also known as nitroglycerin), isosorbide 5-mononitrate, isosorbide dinitrate, pentaerythritol tetranitrate, erythrityl tetranitrate, sodium nitroprusside (SNP), 3-morpholinosydnonimine molsidomine, S-nitroso-N-acetyl penicilliamine (SNAP) S-nitroso-N-glutathione (SNO-GLU), N-hydroxy-L-arginine, amylnitrate, linsidomine, linsidomine chlorohydrate, (SIN-1) S-nitroso-N-cysteine, diazenium diolates, (NONOates), 1,5-pentanedinitrate, L-arginene, ginseng, zizphi fructus, molsidomine, Re-2047, nitrosylated maxisylyte derivatives such as NMI-678-11 and NMI-937 as described in published PCT application WO 0012075.

4) One or more potassium channel openers or modulators. Suitable potassium channel openers/modulators for use herein include nicorandil, cromokalim, levcromakalim, lemakalim, pinacidil, cliazoxide, minoxidil, charybdotoxin, glyburide, 4-amini pyridine, BaCl₂.

5) One or more dopaminergic agents, preferably apomorphine or a selective D2, D3 or D2/D₃agonist such as, pramipexole and ropirinol (as claimed in WO-0023056), PNU95666 (as claimed in WO-0040226).

6) One or more vasodilator agents. Suitable vasodilator agents for use herein include nimodepine, pinacidil, cyclandelate, isoxsuprine, chloroprumazine, halo peridol, Rec 15/2739, trazodone.

7) One or more thromboxane A2 agonists.

8) One or more CNS active agents.

9) One or more ergot alkoloids. Suitable ergot alkaloids are described in U.S. Pat. No. 6,037,346 issued on Mar. 14, 2000 and include acetergamine, brazergoline, bromerguride, cianergoline, delorgotrile, disulergine, ergonovine maleate, ergotamine tartrate, etisulergine, lergotrile, lysergide, mesulergine, metergoline, metergotamine, nicergoline, pergolide, propisergide, proterguride, terguride.

10) One or more compounds which modulate the action of natruretic factors in particular atrial naturetic factor (also known as atrial naturetic peptide), B type and C type naturetic factors such as inhibitors or neutral endopeptidase.

11) One or more compounds which inhibit angiotensin-converting enzyme such as enapril, and combined inhibitors of angiotensin-converting enzyme and neutral endopeptidase such as omapatrilat.

12) One or more angiotensin receptor antagonists such as losartan.

13) One or more substrates for NO-synthase, such as L-arginine.

14) One or more calcium channel blockers such as amlodipine.

15) One or more antagonists of endothelin receptors and inhibitors or endothelin-converting enzyme.

16) One or more cholesterol lowering agents such as statins (e.g. atorvastatin/Lipitor—trade mark) and fibrates.

17) One or more antiplatelet and antithrombotic agents, e.g. tPA, uPA, warfarin, hirudin and other thrombin inhibitors, heparin, thromboplastin activating factor inhibitors.

18) One or more insulin sensitising agents such as rezulin and hypoglycaemic agents such as glipizide.

19) L-DOPA or carbidopa.

20) One or more acetylcholinesterase inhibitors such as donezipil.

21) One or more steroidal or non-steroidal anti-inflammatory agents.

22) One or more estrogen receptor modulators and/or estrogen agonists and/or estrogen antagonists, preferably raloxifene or lasofoxifene, (−)-cis-6-phenyl-5-[4-(2-pyrrolidin-1-yl-ethoxy)-phenyl]-5,6,7,8-tetrahydronaphthalene-2-ol and pharmaceutically acceptable salts thereof the preparation of which is detailed in WO 96/21656.

23) One or more modulators of cannabinoid receptors.

24) One or more of an NPY (neuropeptide Y) inhibitor, more particularly NPY1 or NPY5 inhibitor, preferably NPY1 inhibitor, preferably said NPY inhibitors (including NPY Y1 and NPY Y5) having an IC50 of less than 100 nM , more preferably less than 50 nM. An assay for identifying NPY inhibitors is presented in WO-A-98/52890 (see page 96, lines 2 to 28).

25) One or more of vasoactive intestinal protein (VIP), VIP mimetic, VIP analogue, more particularly mediated by one or more of the VIP receptor subtypes VPAC1,VPAC or PACAP (pituitory adenylate cyclase activating peptide), one or more of a VIP receptor agonist or a VIP analogue (eg Ro-125-1553) or a VIP fragment, one or more of a x-adrenoceptor antagonist with VIP combination (eg Invicorp, Aviptadil).

26) One or more of a melanocortin receptor agonist or modulator or melanocortin ehancer, such as melanotan 11, PT-14, PT-141 or compounds claimed in WO-09964002, WO-00074679, WO-09955679, WO-00105401, WO-00058361, WO-00114879, WO-00113112, WO-09954358.

27) One or more of a serotonin receptor agonist, antagonist or modulator, more particularly agonists, antagonists or modulators for 5HT1A (including VML 670), 5HT2A, 5HT2C, 5HT3 and/or 5HT6 receptors, including those described in WO-09902159, WO-00002550 and/or WO-00028993.

28) One or more of a testosterone replacement agent (inc dehydroandrostendione), testosternone (Tostrelle), dihydrotestosterone or a testosterone implant.

29) One or more of estrogen, estrogen and medroxyprogesterone or medroxyprogesterone acetate (MPA) (i.e. as a combination), or estrogen and methyl testosterone hormone replacement therapy agent (e.g. HRT especially Premarin, Cenestin, Oestrofeminal, Equin, Estrace, Estrofem, Elleste Solo, Estring, Eastraderm TTS, Eastraderm Matrix, Dermestril, Premphase, Preempro, Prempak, Premique, Estratest, Estratest HS, Tibolone).

30) One or more of a modulator of transporters for noradrenaline, dopamine and/or serotonin, such as bupropion, GW-320659.

31) One or more of a purinergic receptor agonist and/or modulator.

32) One or more of a neurokinin (NK) receptor antagonist, including those described in WO-09964008.

33) One or more of an opioid receptor agonist, antagonist or modulator, preferably agonists for the ORL-1 receptor.

34) One or more of an agonist or modulator for oxytocin/vasopressin receptors, preferably a selective oxytocin agonist or modulator.

35) One or more of a PDE inhibitor, more particularly a PDE 2, 3, 4, 5, 7 or 8 inhibitor, preferably PDE2 or PDE5 inhibitor and most preferably a PDE5 inhibitor (see hereinafter), said inhibitors preferably having an IC50 against the respective enzyme of less than 100 nM. Suitable cGMP PDE5 inhibitors for the use according to the present invention include:

the pyrazolo [4,3-d]pyrimidin-7-ones disclosed in EP-A-0463756; the pyrazolo [4,3-d]pyrimidin-7-ones disclosed in EP-A-0526004; the pyrazolo [4,3-d]pyrimidin-7-ones disclosed in published international patent application WO 93/06104; the isomeric pyrazolo [3,4-d]pyrimidin-4-ones disclosed in published international patent application WO 93/07149; the quinazolin-4-ones disclosed in published international patent application WO 93/12095; the pyrido [3,2-d]pyrimidin-4-ones disclosed in published international patent application WO 94/05661; the purin-6-ones disclosed in published international patent application WO 94/00453; the pyrazolo [4,3-d]pyrimidin-7-ones disclosed in published international patent application WO 98/49166; the pyrazolo [4,3-d]pyrimidin-7-ones disclosed in published international patent application WO 99/54333; the pyrazolo [4,3-d]pyrimidin-4-ones disclosed in EP-A-0995751; the pyrazolo [4,3-d]pyrimidin-7-ones disclosed in published international patent application WO 00/24745; the pyrazolo [4,3-d]pyrimidin-4-ones disclosed in EP-A-0995750; the compounds disclosed in published international application W095/19978; the compounds disclosed in published international application WO 99/24433 and the compounds disclosed in published international application WO 93/07124. The pyrazolo [4,3-d]pyrimidin-7-ones disclosed in published international application WO 01/27112; the pyrazolo [4,3-d]pyrimidin-7-ones disclosed in published international application WO 01/27113; the compounds disclosed in EP-A-1092718 and the compounds disclose din EP-A-1092719.

Further suitable PDE5 inhibitors for the use according to the present invention include: 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756); 5-(2-ethoxy-5-morpholinoacetylphenyl)-1-methyl-3-n-propyl-1,6-dihydro-7H-pyrazolo[4,3-d]pyrimidin-7-one (see EP-A-0526004); 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO98/49166); 3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (see WO99/54333); (+)-3-ethyl-5-[5-(4-ethylpiperazin-1-ylsulphonyl)-2-(2-methoxy-1(R)-methylethoxy)pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1 R)-2-methoxy-1-methylethyl]oxy)pyridin-3-yl}-2-methyl-2,6-dihydro-7H-pyrazolo[4,3-d]pyrimidin-7-one (see WO99/54333); 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine (see WO 01/27113, Example 8); 5-[2-iso-Butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one (see WO 01/27113, Example 15); 5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/27113, Example 66); 5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one (see WO01/27112, Example 124); 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one (see WO 01/27112, Example 132); (6R,12aR)-2,3,6,7,12,12a-hexahydro-2-methyl-6-(3,4-methylenedioxyphenyl) -pyrazino[2′,1′:6,1]pyrido[3,4-b]indole-1,4-dione (IC-351), i.e. the compound of examples 78 and 95 of published international application WO95/19978, as well as the compound of examples 1, 3, 7 and 8; 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil) also known as 1-[[3-(3,4-dihydro-5-methyl-4-oxo-7-propylimidazo[5,1 -f]-as-triazin-2-yl)-4-ethoxyphenyl]sulphonyl]-4-ethylpiperazine, i.e. the compound of examples 20, 19, 337 and 336 of published international application WO99/24433; and the compound of example 11 of published international application WO93/07124 (EISAI); and compounds 3 and 14 from Rotella D. P., J. Med. Chem., 2000, 43, 1257.

Still other suitable PDE5 inhibitors include:4-bromo-5-(pyridylmethylamino)-6-[3-(4-chlorophenyl)-propoxy]-3(2H)pyridazinone; 1-[4-[(1,3-benzodioxol-5-ylmethyl)amiono]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1 -b]purin-4(3H)one; furazlocillin; cis-2-hexyl-5-methyl-3,4,5,6a,7,8,9,9a- octahydrocyclopent[4,5]-imidazo[2,1-b]purin-4-one; 3-acetyl-1-(2-chlorobenzyl)-2-propylindole-6- carboxylate; 3-acetyl-1-(2-chlorobenzyl)-2-propylindole-6-carboxylate; 4-bromo-5-(3-pyridylmethylamino)-6-(3-(4-chlorophenyl) propoxy)-3-(2H)pyridazinone; 1-methyl-5(5-morpholinoacetyl-2-n-propoxyphenyl)-3-n-propyl-1,6-dihydro- 7H-pyrazolo(4,3-d)pyrimidin-7-one; 1-[4-[(1,3-benzodioxol-5-ylmethyl)amino]-6-chloro-2- quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E-4010 (Eisai); Bay-38-3045 & 38-9456 (Bayer) and Sch-51866.

If a combination of active agents are administered, then they may be administered simultaneously, separately or sequentially.

The compounds of the invention can be administered alone but, in human therapy will generally be administered in admixture with a suitable pharmaceutical excipient diluent or carrier selected with regard to the intended route of administration and standard pharmaceutical practice.

For example, the compounds of the invention, can be administered orally, buccally or sublingually in the form of tablets, capsules (including soft gel capsules), ovules, elixirs, solutions or suspensions, which may contain flavouring or colouring agents, for immediate-, delayed-, modified-, sustained-, dual-, controlled-release or pulsatile delivery applications. The compounds of the invention may also be administered via fast dispersing or fast dissolving dosage forms.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate modifying excipients may be present both within the dosage form i.e. within the matrix, and/or on the dosage form, i.e. upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used, i.e. where the drug substance is insoluble a fast dispersing dosage form can be prepared and where the drug substance is soluble a fast dissolving dosage form can be prepared.

The compositions of the invention may be administered by direct injection. The composition may be formulated for parenteral, mucosal, intramuscular, intravenous, subcutaneous, ocular, intraocular or transdermal administration. Depending upon the need, the agent may be administered at a dose of from 0.01 to 30 mg/kg body weight, such as from 0.1 to 10 mg/kg, more preferably from 0.1 to 1 mg/kg body weight. p The term “administered” includes delivery by viral or non-viral techniques. Viral delivery mechanisms include but are not limited to adenoviral vectors, adeno-associated viral (AAV) vectos, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof. The routes for such delivery mechanisms include but are not limited to mucosal, nasal, oral, parenteral, gastrointestinal, topical, or sublingual routes.

In addition or in the alternative the compositions (or component parts thereof) of the present invention may be administered by direct injection. In addition or in the alternative the compositions (or component parts thereof) of the present invention may be administered topically (preferably to the genitalia). In addition or in the alternative the compositions (or component parts thereof) of the present invention may be administered by inhalation. In addition or in the alternative the compositions (or component parts thereof) of the present invention may also be administered by one or more of: a mucosal route, for example, as a nasal spray or aerosol for inhalation or as an ingestable solution such as by an oral route, or by a parenteral route where delivery is by an injectable form, such as, for example, by a rectal, ophthalmic (including intravitreal or intracameral), nasal, topical (including buccal and sublingual), intrauterine, vaginal or parenteral (including subcutaneous, intraperitoneal, intramuscular, intravenous, intradermal, intracranial, intratracheal, and epidural) transdermal, intraperitoneal, intracranial, intracerebroventricular, intracerebral, intravaginal, intrauterine, or parenteral (e.g., intravenous, intraspinal, subcutaneous, transdermal or intramuscular) route.

By way of example, the pharmaceutical compositions of the invention may be administered in accordance with a regimen of 1 to 10 times per day, such as once or twice per day. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

Hence, the term “administered” includes but is not limited to delivery by a mucosal route, for example, as a nasal spray or aerosol for inhalation or as an ingestable solution; a parenteral route where delivery is by an injectable form, such as, for example, an intravenous, intramuscular or subcutaneous route.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethyl cellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

The compounds of the invention can also be administered parenterally, for example, intravenously, intra-arterially, intraperitoneally, intrathecally, intraventricularly, intraurethrally intrasternally, intracranially, intramuscularly or subcutaneously, or they may be administered by infusion techniques. In addition, they may be administered in the form of an implant. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art. Parenteral formulations may be formulated for immediate-, delayed-, modified-, sustained-, dual-, controlled-release or pulsatile delivery.

The following dosage levels and other dosage levels herein are for the average human subject having a weight range of about 65 to 70 kg. The skilled person will readily be able to determine the dosage levels required for a subject whose weight falls outside this range, such as children and the elderly.

For oral and parenteral administration to human patients, the daily dosage level of the compounds of the invention or salts or solvates thereof will usually be from 10 to 1000 mg (in single or divided doses).

Thus, for example, tablets or capsules of the compounds of the invention or salts or solvates thereof may contain from 5 to 1000 mg, such as 5 mg to 500 mg of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention. The skilled person will also appreciate that, in the treatment of certain conditions (including FSD), compounds of the invention may be taken as a single dose on an “as required” basis (i.e. as needed or desired).

The compounds of the invention can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A [trade mark] or 1,1,1,2,3,3,3-heptafluoropropane (HFA 227EA [trade mark]), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or “puff” contains from 1 to 50 mg of a compound of the invention for delivery to the patient. The overall daily dose with an aerosol will be in the range of from 1 to 50 mg which may be administered in a single dose or, more usually, in divided doses throughout the day.

Alternatively, compounds of the invention can be administered in the form of a suppository or pessary, or they may be applied topically (preferably to the genitalia) in the form of a gel, hydrogel, lotion, solution, cream, ointment or dusting powder. The compounds of the invention may also be dermally administered. The compounds of the invention may also be transdermally administered, for example, by the use of a skin patch. They may also be administered by the ocular, pulmonary or rectal routes.

For ophthalmic use, compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin (preferably to the genitalia), compounds of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyidodecanol, benzyl alcohol and water.

The compounds of the invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

In a preferred embodiment, the compounds of the invention are delivered systemically (such as orally, buccally and sublingually), more preferably orally. Preferably such systemic (most preferably oral) administration is used to treat female sexual dysfunction, preferably FSAD.

Thus in a particularly preferred embodiment, there is provided the use of the compounds of the invention in the manufacture of a systemically delivered (preferably orally delivered) medicament for the treatment or prophylaxis of FSD, more preferably FSAD.

A preferred oral formulation uses immediate release tablets; or fast dispersing or dissolving dosage formulations (FDDFs).

In a further preferred embodiment, the compounds of the invention are administered topically, preferably directly to the female genitalia, especially the vagina.

Since NEP is present throughout the body, it is very unexpected that the compounds of the invention can be administered systemically and achieve a therapeutic response in the female genitalia without provoking intolerable (adverse) side effects. Thus in the in vivo (rabbit) results hereafter, the compounds of the invention administered systemically increased genital blood flow, upon sexual arousal (mimiced by pelvic nerve stimulation) without adversely affecting cardiovascular parameters, such as causing a significant hypotensive or hypertensive.

Preferably the compounds of the invention are administered for the treatment of FSD in the sexually stimulated patient (by sexual stimulation we mean to include visual, auditory or tactile stimulation).The stimulation can be before, after or during said administration.

Thus the compounds of the invention enhance the pathways/mechanisms that underlie sexual arousal in the female gentialia restoring or improving the sexual arousal response to sexual stimulation.

Thus a preferred embodiment provides the use of a compound of the invention in the preparation of a medicament for the treatment or prophyaxis of FSD in the stimulated patient.

For veterinary use, a compound of the invention, is administered as a suitably acceptable formulation in accordance with normal veterinary practice and the veterinary surgeon will determine the dosing regimen and route of administration which will be most appropriate for a particular animal.

The following formulation examples are illustrative only and are not intended to limit the scope of the invention. “Active ingredient” means a compound of the invention.

Formulation 1: A tablet is prepared using the following ingredients:

| | weight/mg |
|---|---|
| Active ingredient | 250 |
| Cellulose, microcrystalline | 400 |
| Silicon dioxide, fumed | 10 |
| Stearic acid | 5 |
| Total | 665 |

the components are blended and compressed to form tablets.

Formulation 2: An intravenous formulation may be prepared as follows:

| | |
|---|---|
| Active ingredient | 100 mg |
| Isotonic saline | 1,000 ml |

Typical formulations useful for administering the compounds of the invention topically to the genitalia are as follows:

Formualtion 3: A spray

Active ingredient (1.0%) in isopropanol (30%) and water.

Formulation 4: A foam

Active ingredient, acetic acid glacial, benzoic acid, cetyl alcohol, methyl parahydroxybenzoate, phosphoric acid, polyvinyl alcohol, propylene glycol, sodium carboxymethylcellulose, stearic acid, diethyl stearamide, van Dyke perfume No. 6301, purified water and isobutane.

Formulation 5: A gel

Active ingredient, docusate sodium BP, isopropyl alcohol BP, propylene glycol, sodium hydroxide, carbomer 934P, benzoic acid and purified water.

Formulation 6: A Cream

Active ingredient, benzoic acid, cetyl alcohol, lavender, compound 13091, methylparaben, propylparaben, propylene glycol, sodium carboxymethylcellulose, sodium lauryl sulfate, stearic acid, triethanolmine, acetic acid glacial, castor oil, potassium hydroxide, sorbic acid and purified water.

Formulation 7: A pessary

Active ingredient, cetomacrogol 1000 BP, citric acid, PEG 1500 and 1000 and purified water.

The invention additionally includes:

(i) A pharmaceutical composition including a compound of the invention, together with a pharmaceutically acceptable excipient, diluent or carrier.

(ii) A compound of the invention for use as a medicament.

(iii) A method of treating FSD in a mammal including treating said mammal with an effective amount of a compound of the invention.

(iv) An FSD treating pharmaceutical composition comprising a compound of the invention together with a pharmaceutically acceptable excipient, diluent or carrier.

(v) A compound of the invention for treating FSD.

### EXAMPLES

The invention is illustrated by the following non-limiting examples in which the following abbreviations and definitions are used herein below and also throughout the specification:

| Arbacel ® | filter agent |
|---|---|
| br | broad |
| Boc | tert-butoxycarbonyl |
| CDI | carbonyldiimidazole |
| δ | chemical shift |
| d | doublet |
| Δ | heat |
| DCCI | dicyclohexylcarbodiimide |
| DCM | dichloromethane |
| DMF | N,N-dimethylformamide |
| DMPU | 1,3-dimethyl-3,4,5,6-tetrahydro-2(1H)-pyrimidinone |
| DMSO | dimethylsulfoxide |
| ES⁺ | electrospray ionisation positive scan |
| ES⁻ | electrospray ionisation negative scan |
| Ex | Example |
| h | hours |
| HMPA | hexamethylphosphoramide |
| HOBt | 1-hydroxybenzotriazole |
| HPLC | high pressure liquid chromatography |
| m/z | mass spectrum peak |
| min | minutes |
| MS | mass spectrum |
| NMR | nuclear magnetic resonance |
| Prec | precursor |
| Prep | preparation |
| q | quartet |
| s | singlet |
| t | triplet |
| Tf | trifluoromethanesulfonyl |
| TFA | trifluoroacetic acid |
| THF | tetrahydrofuran |
| TLC | thin layer chromatography |
| TMEDA | N,N,N′,N′-tetramethylethylenediamine |
| TS⁺ | thermospray ionisation positive scan |
| WSCDI | 1-(3-dimethylaminopropyl)-3-ethylcarbodhimide |
| | hydrochloride |
| ¹H Nuclear magnetic resonance (NMR) spectra were in all cases consistent with the proposed structures. Characteristic chemical shifts (δ) are given in parts-per-million downfield from tetramethylsilane using conventional abbreviations for designation of major peaks: e.g. s, singlet; d, doublet; t, triplet; q, quartet; m, multiplet; br, broad. The following abbreviations have been used for common solvents: CDCl₃, | |
| # deuterochloroform; DMSO, dimethylsulphoxide. The abbreviation psi square inch and LRMS means low resolution mass spectrometry. Where thin layer chromatography (TLC) has been used it refers to silica gel TLC using silica gel 60 F₂₅₄ plates, R_{f} is the distance travelled by a compound divided by the distance travelled by the solvent front on a TLC plate. | |

¹H Nuclear magnetic resonance (NMR) spectra were in all cases consistent with the proposed structures. Characteristic chemical shifts (6) are given in parts-per-million downfield from tetramethylsilane using conventional abbreviations for designation of 5 major peaks: e.g. s, singlet; d, doublet; t, triplet; q, quartet; m, multiplet; br, broad. The following abbreviations have been used for common solvents: CDCl₃, deuterochloroform; DMSO, dimethylsulphoxide. The abbreviation psi means pounds per square inch and LRMS means low resolution mass spectrometry. Where thin layer chromatography (TLC) has been used it refers to silica gel TLC using silica gel 60 F₂₅₄ plates, R_{f} is the distance travelled by a compound divided by the distance travelled by the solvent front on a TLC plate.

The powder X-ray diffraction (PXRD) patterns were determined using a Siemens D5000 powder X-ray diffractometer fitted with a theta-theta goniometer, automatic beam divergence slits, a secondary monochromator and a scintillation counter. The specimen was rotated whilst being irradiated with copper K-alpha1 X-rays (Wavelength=1.5046 Angstroms) filtered with a graphite monochromator (λ=0.15405 nm) with the X-ray tube operated at 40 kV/40 mA. The main peaks (in degrees 20) of the PXRD patterns for the various solid forms are illustrated.

### Example 1

### 2-({1-[(1,3-Benzodioxol-5-ylamino)carbonyl]cyclopentyl}methyl)pentanoic acid

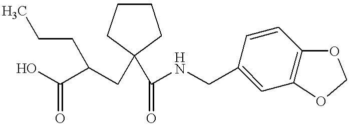

Trifluoroacetic acid (5 ml) was added to a solution of the tert-butyl ester from preparation 34 (130 mg, 0.31 mmol) in dichloromethane (5 ml), and the solution stirred at room temperature for 4 hours. The reaction mixture was concentrated under reduced pressure and the residue azeotroped with toluene and dichloromethane to give the title compound as a clear oil, 112 mg, ¹H NMR (CDCl₃, 400 MHz) δ 0.83 (t, 3H), 1.22-1.40 (m, 3H), 1.50-1.72 (m, 8H), 1.95 (m, 1H), 2.10 (m, 2H), 2.19 (m, 1H), 4.30 (m, 2H), 5.93 (s, 2H), 5.99 (bs, 1H), 6.74 (m, 3H); LRMS: m/z 380 (MH⁻).

### Examples 2 to 9

Compounds of formula Ic, i.e. compounds of general formula I where R¹ is propyl, were prepared from the corresponding tert-butyl ester following a similar procedure to that described in Example 1 from the precursor indicated.

| | |
|---|---|
| | (Ic) |
| 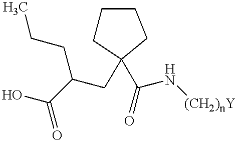 | |

| Ex | Prec | n | Y | Data |
|---|---|---|---|---|
| 2¹ | Prep 35 | 0 | 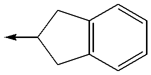 | ¹H NMR (CDCl₃, 400 MHz) δ: 0.81 (s, 3H), 1.17-2.04 (m, 14H), 2.27-2.38 (m, 1H), 2.64-2.80 (m, 2H), 3.20-3.31 (m, 2H), 4.60-4.72 (m, 1H), 5.97 (d, 1H), 7.03-7.18 (m, 4H). LRMS: m/z 343.8 (M⁺). |
| 3^{2,3} | Prep 36 | 0 | 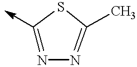 | ¹H NMR (CDCl₃, 400 MHz) δ: 0.90 (t, 3H), 1.30-1.42 (m, 4H), 1.59-1.81 (m, 7H), 2.18 (m, 1H), 2.30 (m, 1H), 2.42 (m, 1H), 2.55 (m, 1H), 2.61 (s, 3H). LRMS: m/z 324 (MH⁻). Mp 184-186°C.; Anal. Found: C, 55.50; H, 7.22; N, |
| # 12.61. C₁₅H₂₃N₃O₃S requires C, 55.36; H, 7.14; N, 12.91%. | | | | |
| 4³ | Prep 37 | 0 | 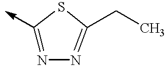 | ¹H NMR (CDCl₃, 400 MHz) δ: 0.92 (t, 3H), 1.35 (t, 3H), 1.25-1.80 (m, 11H), 2.20-2.50 (m, 4H), 2.95 (q, 2H), 12.10 (bs, 1H); LRMS: m/z 339.8 (MH⁺) Anal. Found: C, 56.46; H, 7.46; N, 12.36. C₁₆H₂₅N₃O₃S requires C, 56.62; H, 7.44; N, 12.37%. |
| 5² | Prep 38 | 1 | 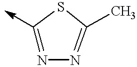 | ¹H NMR (CDCl₃, 400 MHz) δ: 0.80 (t, 3H), 1.20-1.70 (m, 11H), 1.90-2.20 (m, 3H), 2.25 (m, 1H), 2.70 (s, 3H), 4.75 (m, 2H), 7.10 (bs, 1H). LRMS: m/z 340.6 (MH⁺) |
| 6² | Prep 39 | 2 | 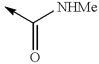 | ¹H NMR (CDCl₃, 400 MHz) δ: 0.88 (t, 3H), 1.25-1.40 (m, 3H), 1.41-1.70 (m, 8H), 1.92 (m, 1H), 2.00-2.18 (m, 2H), 2.38 (m, 1H), 2.42 (t, 2H), 2.80 (d, 3H), 3.40-3.60 (m, 2H), 6.50 (bs, 1H), 6.74 (bs, 1H). LRMS: m/z 313.2 (MH⁺) |
| 7 | Prep 40 | 0 | 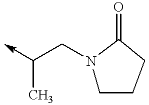 | ¹H NMR (CDCl₃, 400 MHz) δ: 0.85 (t, 3H), 1.19 (d, 3H), 1.21-1.69 (m, 11H), 1.89-2.10 (m, 5H), 2.30 (m, 1H), 2.41 (m, 2H), 2.95 (m, 1H), 3.35 (m, 1H), 3.63 (m, 2H), 4.20 (m, 1H), 6.58-6.70 (m, 1H). LRMS: m/z 353.1 (MH⁺) |
| 8 | Prep 41 | 0 | 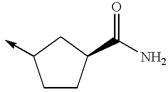 | ¹H NMR (CDCl₃, 400 MHz) δ: 0.81 (t, 3H), 1.20-1.39 (m, 3H), 1.41-2.10 (m, 1H), 2.80 (m, 1H), 4.35 (m, 17H), 5.81 (d, 1H), 6.30 (bs, 0.5H), 6.43 (bs, 0.5H), 7.40 (bd, 0.5H), 7.61 (bd, 0.5H). LRMS: m/z 339.8 (MH⁺) |
| 9 | Prep 32 | 0 | 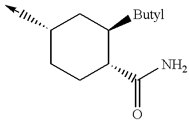 | ¹H NMR (CDCl₃, 400 MHz) δ: 0.84 (m, 6H), 1.08-2.08 (m, 29H), 4.29 (m, 1H), 5.95 (d, 1H), 6.43 (s, 1H), 7.80 (d, 1H). LRMS: m/z 409.5 (MH⁺) |

¹= additionally purified by column chromatography on silica gel using ethyl acetate:pentane as eluant.
²= additionally purified by column chromatography on silica gel using dichloromethane:methanol as eluant.
³= recrystallised from ether

### Example 10

### 2-{[1-({[2-(1H-Indol-3-yl)ethyl]amino}carbonyl)cyclopentyl]methyl}pentanoic acid

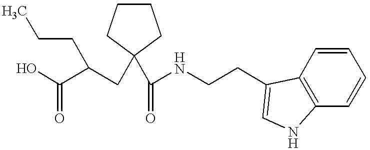

Trifluoroacetic acid (2.61 ml, 33.9 mmol) was added to a solution of the tert-butyl ester from preparation 44 (482 mg, 1.13 mmol) and anisole (1.23 ml, 11.3 mmol) in dichloromethane (4 ml), and the reaction stirred at room temperature for 4 hours. The mixture was washed with water, then brine, dried (MgSO₄), concentrated under reduced pressure and the residue azeotroped with toluene. The residual brown oil was purified by column chromatography on silica gel using dichloromethane:methanol (95:5) as eluant, and re-columned using an elution gradient of ethyl acetate:pentane (30:70 to 50:50) to afford the title compound as a clear foam, 136 mg, 32%; ¹HNMR (CDCl₃, 400 MHz) δ: 0.82 (s, 3H), 1.16-1.77 (m, 12H), 1.78-2.03 (m, 2H), 2.36 (m, 1H), 2.97 (m, 2H), 3.61 (m, 2H), 5.83 (m, 1H), 7.04 (s, 1H), 7.09-7.23 (m, 2H), 7.39 (d, 1H), 7.61 (d, 1H), 8.15 (m, 1H); LRMS: m/z 371.8 (MH⁺).

### Example 11

### 2-{[1-({[3S)-1-Benzylpyrrolidinyl]amino}carbonyl)cyclopentyl]methyl}pentanoic acid

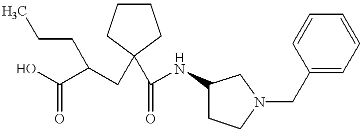

A solution of the tert-butyl ester from preparation 45 (70 mg, 0.16 mmol) in trifluoroacetic acid (1 ml) and dichloromethane (1 ml) was stirred at room temperature for 2 hours. The reaction was concentrated under reduced pressure and the residue azeotroped with dichloromethane. The residue was partitioned between water (1 ml) and ethyl acetate (5 ml), and the pH of the aqueous layer adjusted to 6 using sodium bicarbonate solution. The layers were separated, the organic phase dried (Na₂SO₄), evaporated under reduced pressure and the residue azeotroped with dichloromethane to give the title compound as a beige foam, 45 mg, 73%; ¹H NMR (CDCl₃, 400 MHz) δ: 0.84 (t, 3H), 1.20-2.95 (m, 19H), 3.52 (m, 1H), 3.75 (m, 1H), 3.95 (m, 1H), 4.25 (m, 1H), 4.45 (m, 1H), 6.96 (bs, 1H), 7.39 (m, 5H); LRMS: m/z 387 (MH⁺);

Anal. Found: C, 61.11; H, 7.69; N, 6.00. C₂₃H₃₄N₂O₃; CH₂Cl₂ requires C, 61.14; H, 7.70; N, 5.94%.

### Example 12

### 2-{[1-(}[1-(Hydroxymethyl)cyclopentyl]amino}carbonyl)cyclopentyl]-methyl}pentanoic acid

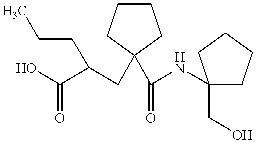

A solution of the tert-butyl ester from preparation 33 (38 mg, 0.1 mmol) in trifluoroacetic acid (2 ml) and dichloromethane (2 ml) was stirred at room temperature for 2 hours. The reaction was concentrated under reduced pressure and the residue azeotroped with toluene and then dichloromethane to give a colourless gum. This was suspended in a solution of potassium carbonate (50 mg, 0.3 mmol) in methanol, and the mixture stirred for 2 hours at room temperature. The methanol was removed under reduced pressure, the residual aqueous mixture diluted with water (20 ml), and acidifed to pH 2 using 2N hydrochloric acid. This solution was extracted with ethyl acetate (2×20 ml), and the combined organic solutions dried (MgSO₄), and evaporated under reduced pressure to give a clear oil, 32 mg, 97%; ¹H NMR (CDCl₃, 400 MHz) δ: 0.88 (t, 3H), 1.20-1.40 (m, 3H), 1.41-1.90 (m, 17H), 2.01-2.20 (m, 2H), 2.40 (m, 1H), 3.71 (dd, 2H), 5.80 (bs, 1H); LRMS m/z 326.1 (MH⁺).

### Example 13

### Cis-2-{[1-({[4-(Hydroxymethyl)cyclohexyl]amino}carbonyl)cyclopentyl]methyl}pentanoic acid

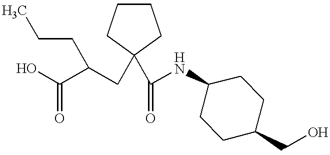

The title compound was obtained as a colourless gum in 68%, from the tert-butyl ester from preparation 43, following the procedure described in example 12, except the product was additionally purified by column chromatography on silica gel using dichloromethane:methanol (95:5) as the eluant; ¹H NMR (CDCl₃, 400 MHz) δ: 0.87 (t, 3H), 1.21-1.40 (m, 6H), 1.52-1.70 (m, 15H), 1.92-2.11 (m, 3H), 2.39 (m, 1H), 3.55 (d, 2H), 4.01 (m, 1H), 5.90 (m, 1H); LRMS: m/z 340.3 ( MH⁺).

### Example 14

### 2-{[1-({[2-(2-Oxo-1-piperidinyl)ethyl]amino}carbonyl)cyclopentyl]methyl}pentanoic acid

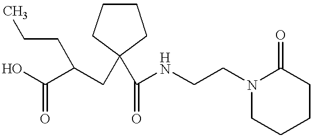

Hydrogen chloride gas was bubbled through an ice-cold solution of the tert-butyl ester from preparation 47 (43 mg, 0.105 mmol) in dichloromethane (10 ml) for 20 minutes. The solution was then stirred at room temperature for 3 hours. The mixture was concentrated under reduced pressure and the residue azeotroped with dichloromethane (3×), to give a glass-like solid. The crude product was purified by column chromatography on silica gel using an elution gradient of dichloromethane:methanol (95:5 to 90:10) to afford the title compound, 6 mg; ¹H NMR (CDCl₃, 400 MHz) δ: 0.81 (t, 3H), 1.20-1.36 (m, 4H), 1.41-1.69 (m, 7H), 1.79 (m, 4H), 1.90-2.10 (m, 3H), 2.30 (m, 1H), 2.38 (t, 2H), 3.30-3.60 (m, 6H), 7.00 (bs, 1H); LRMS: m/z 351 (M−H)⁻.

### Example 15

### 2-({1-[({3-[(Dimethylamino)carbonyl]cyclohexyl}amino)carbonyl]-cyclopentyl}methyl) pentanoic acid

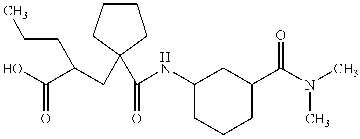

The title compound was obtained as a solid in 85% yield from the tert-butyl ester from preparation 42, following a similar method to that described in example 14, except that dichloromethane:methanol:acetic acid (95:3:2) was used as the chromatographic eluant; ¹H NMR (CDCl₃, 400 MHz) δ: 0.89 (t, 3H), 1.09-1.76 (m, 12H), 1.80-2.17 (m, 10H), 2.37 (m,1H), 2.68 (m,1H), 2.95 (s, 3H), 3.04 (s, 3H), 3.83 (m, 1H), 6.06 (m,1H); LRMS: m/z 381 (MH⁺);

Anal. Found: C, 63.31; H, 9.17; N, 6.53. C₂₁H₃₆N₂O₄; H₂O requires C, 63.29; H, 9.61; N, 7.03%.

### Example 16

### 2-{[1-({[(1R,2R)-2-Phenylcyclopropyl]amino}carbonyl)cyclopentyl]-methyl}pentanoic acid

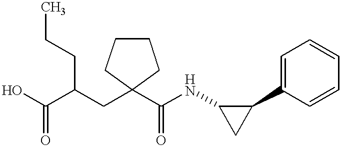

The title compound was obtained quantitatively as an orange gum from the tert-butyl ester from preparation 46, following a similar procedure to that described in example 14; ¹H NMR (CDCl₃, 400 MHz) δ: 0.90 (t, 3H), 1.12-2.14 (m, 17H), 2.38 (m, 1H), 2.87 (m, 1H), 6.10 (s, 1H), 7.13 (m, 3H), 7.25 (m, 2H); LRMS: m/z 344.3 (MH⁺).

### Example 17

### (2R)-2-{[1-({[5-(Cyclopropylmethyl)-1,3,4-thiadiazol-2-yl]amino}carbonyl)-cyclopentyl]methyl}pentanoic acid

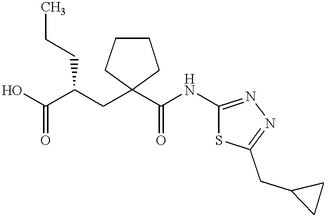

A solution of the tert-butyl ester from preparation 50 (63 mg, 0.15 mmol) in trifluoroacetic acid (2 ml) and dichloromethane (2 ml), was stirred at room temperature for 2 hours. The mixture was concentrated under reduced pressure and the residue purified by column chromatography on silica gel using dichloromethane:methanol (95:5) as eluant to give the title compound as a white foam, 46 mg, 83%; ¹H NMR (CD₃OD, 400 MHz) δ: 0.38 (m, 2H), 0.62 (m, 2H), 0.82 (t, 3H), 1.12 (m, 1H), 1.26 (m, 2H), 1.38 (m, 1H), 1.52 (m, 1H), 1.78-1.78 (m, 6H), 1.90 (m, 1H), 2.23 (m, 4H), 2.92 (d, 2H); LRMS: m/z 366.0 (MH⁺); [α]_{D}=−7.75° (c=0.08, methanol).

### Example 18

### (2R)-2-{[1-({[5-(Ethoxymethyl)-1,3,4-thiadiazol-2-yl]amino}carbonyl)cyclopentyl]-methyl}pentanoic acid

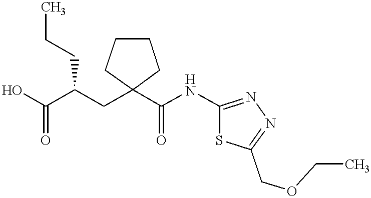

The title compound was obtained as a white foam in 62% yield from the tert-butyl ester from preparation 51, following a similar procedure to that described in example 17; ¹H NMR (CD₃OD, 400 MHz) δ: 0.82 (t, 3H), 1.21-1.40 (m, 7H), 1.50 (m, 1H), 1.60-1.77 (m, 7H), 1.88 (m, 1H), 2.23 (m, 4H), 3.62 (q, 2H); [α]_{D}=−6.08°(c=0.25, methanol).

### Example 19

### 2-({1-[(3-Pyridinylamino)carbonyl]cyclopentyl}methyl)pentanoic acid

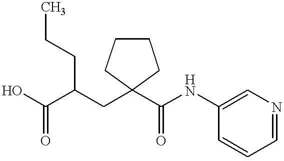

A mixture of the benzyl ester from preparation 52 (130 mg, 0.33 mmol) and 10% palladium on charcoal (20 mg) in 95% aqueous ethanol (3 ml) was hydrogenated at 15 psi and room temperature for 2 hours. The reaction was filtered through Arbocel®, washing through with ethanol, and the filtrate evaporated under reduced pressure. The residual gum was purified by column chromatography on silica gel using dichloromethane:methanol (95:5) as eluant to afford the title compound, 103 mg, 83%; ¹H NMR (CDCl₃, 400 MHz) δ: 0.90 (t, 3H), 1.38 (m, 2H), 1.44 (m, 1H), 1.58-1.82 (m, 8H), 2.19 (m, 1H), 2.39 (m, 2H), 2.52 (m, 1H), 6.88 (m, 1H), 7.67 (m, 1H), 7.82 (d, 1H), 8.38 (d, 1H), 9.78 (s, 1H); LRMS: m/z 305 (MH⁺).

### Example 20

### 2-[(1-{[(4-Butyl-2-pyridinyl)amino]carbonyl}cyclopentyl)methyl]pentanoic acid

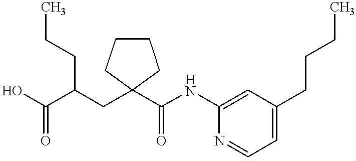

The title compound was obtained in 92% yield from the benzyl ester from preparation 55, following a similar procedure to that described in example 19; ¹H NMR (CDCl₃, 400 MHz) δ: 0.90 (m, 6H), 1.28-1.50 (m, 5H), 1.58-1.81 (m, 1OH), 2.20 (m, 1H), 2.40 (m, 2H), 2.58 (m, 3H), 6.70 (d, 1H), 7.68 (d, 1H), 8.22 (s, 1H), 9.90 (bs, 1H).

### Example 21

### 2-({1-[(3-Benzylanilino)carbonyl]cyclopentyl}methyl)pentanoic acid

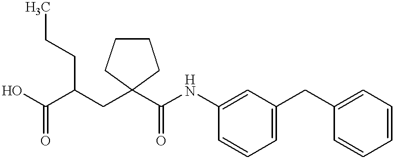

A mixture of the benzyl ester from preparation 53 (1.3 mg, 2.47 mmol) and 5% palladium on charcoal (130 mg) in water (10 ml) and ethanol (40 ml) was hydrogenated at 30 psi and room temperature for 2 hours. The reaction mixture was filtered through Arbocel®, the filtrate concentrated under reduced pressure, and the residue triturated with dichloromethane. The residual gum was triturated with ether, then hexane, and dried at 50° C., to give the title compound as a solid, 0.79 g, 81%; ¹H NMR (CDCl₃, 300 MHz) δ: 0.95 (t, 3H), 1.24-1.51 (m, 3H), 1.58-1.80 (m, 7H), 1.88 (dd, 1H), 2.15 (m, 2H), 2.24 (m, 1H), 2.48 (m, 1H), 4.00 (s, 2H), 6.98 (d, 1H), 7.24 (m, 6H), 7.40 (m, 3H);

Anal. Found: C, 75.48; H, 7.76; N, 3.59. C₂₅H₃₁NO₃; 0.25H₂O requires C, 75.44; H, 7.98; N, 3.51%.

### Example 22

### 2-[(1-[{(1-Benzyl-6-oxo-1,6-dihydro-3-pyridinyl)amino]carbonyl}-cyclopentyl)-methyl]pentanoic acid

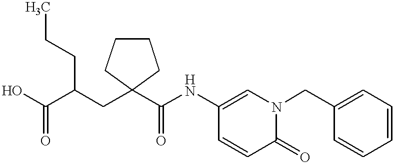

The title compound was obtained as a white foam in 51% yield from the benzyl ester from preparation 56, following a similar procedure to that described in example 21, except the product was purified by column chromatography on silica gel, using ethyl acetate as eluant; ¹H NMR (CDCl₃, 300 MHz) δ: 0.96 (t, 3H), 1.28-1.80 (m, 12H), 2.01 (m, 1H), 2.30-2.52 (m, 2H), 5.02 (dd, 2H), 6.60 (d, 1H), 7.27 (m, 5H), 7.70 (s, 1H), 8.34 (s, 1H);

Anal. Found: C, 69.52; H, 7.41; N, 6.51. C₂₄H₃₀N₂O₄; 0.25H₂O requires C, 69.45; H, 7.41; N, 6.75.

### Example 23

### cis-2-({1-[({4-[(Dimethylamino)carbonyl]cyclohexyl}amino)carbonyl]-cyclopentyl}methyl)pentanoic acid

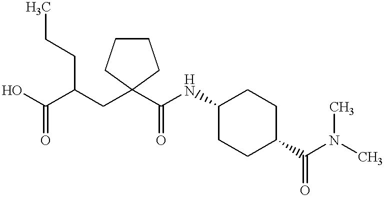

A mixture of the benzyl ester from preparation 58 (150 mg, 0.33 mmol) and 10% palladium on charcoal (20 mg) in water (0.3 ml) and ethanol (3.5 ml) was hydrogenated at 15 psi and room temperature for 3 days. The reaction mixture was filtered through Arbocel®, and the filtrate concentrated under reduced pressure. The residual gum was purified by column chromatography on silica gel using dichloromethane:methanol (95:5) as eluant to afford the title compound, 85 mg, 65%; ¹H NMR (CDCl₃, 400 MHz) δ: 0.84 (t, 3H), 1.29-1.96 (m, 18H), 2.01-2.23 (m, 4H), 2.37 (m, 1H), 2.62 (m, 1H), 2.96 (s, 3H), 3.03 (s, 3H), 3.96 (m, 1H), 5.98 (m, 1H); LRMS: m/z 381.8 (MH⁺);

Anal. Found: C, 63.81; H, 9.58; N, 6.99. C₂₁H₃₆N₂O₄; 0.2CH₂Cl₂ requires C, 64.06; H, 9.23; N, 7.05%.

### Example 24

### cis-2-({1-[({4-[(Methylamino)carbonyl]cyclohexyl}amino)carbonyl]cyclopentyl}-methyl)pentanoic acid

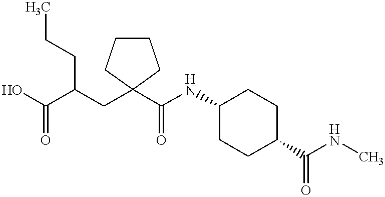

The title compound was obtained as a white solid in 34% yield from the benzyl ester from preparation 59, following the procedure described in example 23; ¹H NMR (CDCl₃, 300 MHz) δ: 0.90 (t, 3H), 1.26-2.02 (m, 20H), 2.19 (m, 3H), 2.39 (m, 1H), 2.82 (d, 3H), 4.00 (m, 1H), 5.69 (m, 1H), 6.00 (d, 1H); LRMS: m/z 365 (M−H⁻).

### Example 25

### 2-[(1-{[(5-Benzyl-3-pyridinyl)amino]carbonyl}cyclopentyl)methyl]pentanoic acid

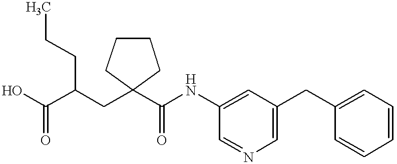

A mixture of the benzyl ester from preparation 54 (850 mg, 1.76 mmol) and 5% palladium on charcoal (100 mg) in 20% aqueous ethanol (30 ml) was hydrogenated at 30 psi and room temperature for 2 hours. The mixture was filtered through Arbocel®, the filtrate evaporated under reduced pressure, and the residue azeotroped with dichloromethane to give the title compound as a foam, 0.63 g; ¹H NMR (CDCl₃, 300 MHz) δ: 0.92 (t, 3H), 1.30-1.83 (m, 11H), 2.07 (m, 1H), 2.42 (m, 3H), 3.82 (s, 2H), 7.15-7.38 (5H), 7.80 (s, 1H), 8.48 (s, 1H), 8.59 (s, 1H), 8.62 (s, 1H);

Anal. Found: C, 72.29; H, 7.70; N, 6.90. C₂₄H₃₀N₂O₃; 0.25H₂O requires C, 72.24; H, 7.70; N, 7.02%.

### Example 26

### 2-({1-[({1-Benzyl-2-oxo-2-[(3-pyridinylsulfonyl)amino]ethyl}amino)-carbonyl]cyclopentyl}methyl)pentanoic acid

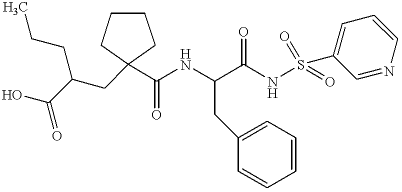

A mixture of the benzyl ester from preparation 57 (918 mg, 1.52 mmol) and 10% palladium on charcoal (90 mg) in water (10 ml) and ethanol (50 ml) was hydrogenated at 50 psi and room temperature for 4Y/2 hours. Tlc analysis showed starting material remaining, so additional catalyst (70 mg) was added, and the mixture hydrogenated for a further 18 hours. Tlc analysis, again showed starting material remaining, so further catalyst (70 mg) was added, and hydrogenation continued for an additional 6 hours. The reaction mixture was filtered through Arbocel®, the filtrate evaporated under reduced pressure and the residue azeotroped with dichloromethane. The crude product was purified by column chromatography on silica gel using an elution gradient of dichloromethane:acetic acid:ethanol (99:1:0 to 79.1:0.9:20) to afford the title compound as a white foam, 271 mg, 35%; ¹H NMR (DMSOd₆, 300 MHz) δ: 0.75 (m, 3H), 0.96-1.42 (m, 11H), 1.61-1.99 (m, 4H), 2.75-3.02 (m, 2H), 4.45 (m, 1H), 7.20 (m, 6H), 7.62 (m, 1H), 8.24 (m, 1H), 8.83 (s, 1H), 9.01 (s, 1H), 11.98 (bs, 1H), 12.70 (bs, 1H); IR (KBr disc) 1185, 1195 (m), 1455, 1515, 1640, 1704, 2870, 2930, 2960 (s).

### Example 27

### 2-({1-[({2-[(Phenylsulfonyl)amino]ethyl}amino)carbonyl]cyclopentyl}-methyl)pentanoic acid

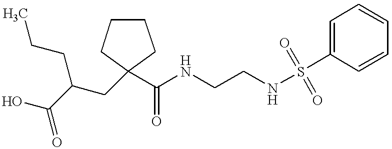

A mixture of the amine from preparation 61 (235 mg, 0.72 mmol), benzenesulphonyl chloride (127 mg, 0.72 mmol) and triethylamine (150 μl, 1.08 mmol) in dichloromethane (6 ml) was stirred at room temperature for 2 days. The mixture was concentrated under reduced pressure and the residue purified by column chromatography on silica gel using ethyl acetate:pentane (30:70) as eluant to give a clear oil. This was then dissolved in trifluoroacetic acid (3 ml) and dichloromethane (3 ml) and the solution stirred at room temperature for 6 hours. The mixture was concentrated under reduced pressure and the residue azeotroped twice with toluene. The crude product was purified by column chromatography on silica gel using ethyl acetate:pentane (30:70) to afford the title compound as a clear oil, 204 mg, 69%; ¹H NMR (CDCl₃, 400 MHz) δ: 0.84 (t, 3H), 1.22-1.43 (m, 4H), 1.43-2.18 (m, 1OH), 2.36 (m, 1H), 3.11 (m, 2H), 3.20-3.31 (m, 1H), 3.42-3.53 (m, 1H), 6.13-6.24 (m, 1H), 7.42-7.59 (m, 3H), 7.84 (m, 2H); LRMS: m/z 411.8 (MH⁺);

Anal. Found: C, 57.26; H, 7.40; N, 6.61. C₂₀H₃₀N₂O₅S requires C, 57.18; H, 7.22; N, 6.62%.

### Example 28

### 2-({1-[({2-[(Benzylsulfonyl)amino]ethyl}amino)carbonyl]cyclopentyl}-methyl)pentanoic acid

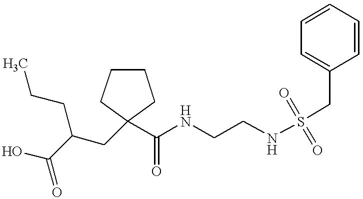

The title compound was obtained as a clear oil in 97% yield, from the amine from preparation 61, following the procedure described in example 27, ¹H NMR (CDCl₃, 300 MHz) δ: 0.87 (t, 3H), 1.19-1.72 (m, 11H), 1.80-1.96 (m, 1H), 2.00-2.16 (m, 2H), 2.27-2.38 (m, 1H), 2.92-3.21 (m, 3H), 3.23-3.39 (m, 1H), 4.25 (s, 2H), 5.80-6.06 (m, 1H), 6.38 (m, 1H), 7.29-7.43 (m, 5H); LRMS: m/z 425.8 (MH⁺).

### Example 29

### (2R)-2-[(1-{[(5-Ethyl-1,3,4-thiadiazol-2-yl)amino]carbonyl}cyclopentyl) methyl]pentanoic acid

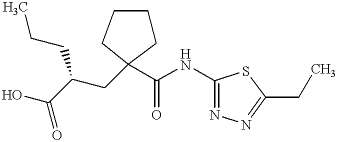

and

### Example 30

### (2S)-2-[(1-{[(5-Ethyl-1,3,4-thiadiazol-2-yl)amino]carbonyl}cyclopentyl) methyl]pentanoic acid

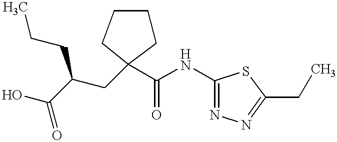

The acid from Example 4 (824 mg) was further purified by HPLC using an AD column and using hexane:iso-propanol:trifluoroacetic acid (85:15:0.2) as eluant to give the title compound of example 29 as a white foam, 400 mg, 99.5% ee, ¹H NMR (CDCl₃, 400 MHz) δ: 0.90 (t, 3H), 1.36 (m, 6H), 1.50-1.80 (m, 9H), 2.19 (m, 1H), 2.30 (m, 1H), 2.44 (m, 1H), 2.60 (m, 1H), 2.98 (q, 2H), 12.10-12.30 (bs, 1H), LRMS: m/z 338 (MH⁻), [α]_{D}=−9.0°(c=0.1, methanol), and the title compound of example 30 as a white foam, 386 mg, 99% ee, ¹H NMR (CDCl₃, 400 MHz) δ: 0.90 (t, 3H), 1.38 (m, 6H), 1.50-1.79 (m, 9H), 2.19 (m, 1H), 2.30 (m, 1H), 2.44 (m, 1H), 2.60 (m, 1H), 2.98 (q, 2H), 12.10-12.27 (bs, 1H);

LRMS: m/z 338 (MH⁻); and [α]_{D}=+3.8°(c=0.1, methanol).

Alternatively, Example 29 may be prepared as follows:

To a solution of the product from Preparation 51a (574 g, 1.45 mol) in dichloromethane (2.87 L) was added trifluoroacetic acid (1.15 L) over a period of 50 minutes with cooling at 10° C. After addition was complete, the reaction was allowed to warm to ambient temperature with stirring under a nitrogen atmosphere for 24 hours. Deionised water (2.6 L) was then added. The reaction mixture was then washed with deionised water (3×2.6 L). The dichloromethane layer was concentrated to a volume of approximately 1 L to give the crude title compound (439 g, 1.29 mol, 96% yield) as a solution in dichloromethane. A purified sample of the title compound was obtained using the following procedure. To a dichloromethane solution (2.34 L) of the crude product, that had been filtered to remove any particulate contamination, was added isopropyl acetate (1.38 L). The resultant mixture was distilled at atmospheric pressure whilst being simultaneously replaced with isopropyl acetate until the solution temperature reached 87° C. The heating was stopped and the solution was allowed to cool to ambient temperature with stirring for 14 hours to give a cloudy brown solution. The agitation rate was then increased and crystallisation commenced. The suspension was then allowed to granulate for 12 hours at ambient temperature. The resultant suspension was then cooled to 0° C. for 3.5 hours and the solid was then collected by filtration. The filter cake was then washed with isopropyl acetate (2×185 ml, then 2×90 ml) and the solid was dried under vacuum at 40-45° C. for 18 hours to give the title compound (602 g, 0.18 mol, 70% yield) as a cream coloured, crystalline solid; m.p.: 130-136° C.; LRMS (negative APCI): m/z [M−H]⁻ 338; ¹H-NMR (CDCl₃, 300 MHz) δ: 0.92 (t, 3H), 1.27-1.52 (m, 7H), 1.52-1.89 (m, 8H), 2.11-2.27 (m, 1H), 2.27-2.37 (m, 1H), 2.42-2.55 (m, 1H), 2.65 (dd, 2H), 3.00 (q, 2H), 12.25 (bs, 1H).

Example 29 may be purified as follows:

The title product from Example 29 was disolved in methanol. To this solution was added sodium methoxide (1 equivalent) in methanol (1 ml/g of Example 29) and the mixture was stirred at room temperature for 20 minutes. The solvent was removed in vacuo and the residue was azeotoped with ethyl acetate to give a brown residue. Ethyl acetate was added and the solution filtered to give a brown solid which was washed with tert-butylmethyl ether to give the crude sodium salt of Example 29. This crude product (35 g) was partitioned between water (200 ml) and ethyl acetate (350 ml). Concentrated hydrochloric acid (˜7 ml) was added until the pH of the aqueous layer was pH2. The aqueous phase was washed with ethyl acetate (2×100 ml). The combined layers were dried using magnesium sulphate. The solvent was removed in vacuo to give a light brown solid (31 g). Ethyl acetate (64 ml, 2 ml/g) and diisopropyl ether (155 ml, 5 ml/g) were added and the mixture heated to 68° C. until a clear solution was obtained (˜30 min). Upon cooling to room temperature, crystallisation of the free acid occurred. After 30 minutes stirring at room temperature the product was collected by filtration and washed with diisopropyl ether. The product was dried in a vacuum oven at 50° C. overnight. (20.2 g, 61% recovery from the sodium salt.); m.p. 135 degC (determined using a Perkin Elmer DSC7 at a heating rate of 20° C./minute).

The main peaks (from 2 to 40 degrees 2θ) in the PXRD pattern are as follows. The PXRD was performed without reference to an internal standard (e.g. silicon powder). The peak positions are therefore subject to possible instrumental zero offset and sample height errors.

| Angle | Intensity | Angle | Intensity | Angle | Intensity |
|---|---|---|---|---|---|
| °2-Theta | % | °2-Theta | % | °2-Theta | % |
| 6.701 | 59.8 | 20.121 | 36.1 | 28.539 | 17.3 |
| 8.908 | 11.2 | 20.425 | 7.1 | 30.092 | 56.0 |
| 9.305 | 8.6 | 21.104 | 8.9 | 31.236 | 8.8 |
| 9.590 | 4.5 | 21.326 | 5.7 | 32.010 | 12.8 |
| 10.039 | 25.3 | 21.733 | 100.0 | 32.810 | 8.0 |
| 10.206 | 59.3 | 22.150 | 18.7 | 33.106 | 6.6 |
| 10.578 | 11.3 | 22.816 | 10.2 | 33.720 | 8.0 |
| 10.984 | 12.2 | 23.327 | 46.2 | 34.033 | 9.2 |
| 12.075 | 10.3 | 23.681 | 6.4 | 34.388 | 12.4 |
| 13.054 | 5.5 | 23.960 | 9.5 | 34.646 | 8.5 |
| 14.256 | 8.2 | 24.248 | 14.0 | 34.821 | 8.2 |
| 15.695 | 45.2 | 24.722 | 9.4 | 35.806 | 7.3 |
| 16.316 | 17.1 | 25.505 | 15.5 | 36.272 | 13.5 |
| 16.418 | 8.5 | 25.779 | 10.5 | 36.633 | 12.1 |
| 16.698 | 24.1 | 26.267 | 7.0 | 37.500 | 7.8 |
| 17.129 | 16.5 | 26.724 | 20.4 | 37.862 | 11.7 |
| 17.605 | 20.9 | 26.890 | 70.4 | 38.281 | 6.5 |
| 18.575 | 23.6 | 27.663 | 7.2 | 39.975 | 11.8 |
| 19.296 | 14.0 | 28.158 | 16.8 | | |

Salts of Example 29

Sodium Salts

a) Lower Melting Form

The title product from Example 29 was dissolved in methanol (80 mg in 2.5 mls-33 ml/g). Sodium hydroxide (0.024 mls as 10N solution) was added. On evaporation of the methanol crystallisation of the sodium salt occurred, which was used with no further purification. The salt was dried on high vacuum for 5 hrs to give 85 mg, quantitative yield; m.p. 214 degC (determined using a TA instruments DSC2910 at a heating rate of 10° C./minute).

The main peaks (from 2 to 40 degrees 20) in the PXRD pattern are as follows. The PXRD was performed without reference to an internal standard (e.g. silicon powder). The peak positions are therefore subject to possible instrumental zero offset and sample height errors.

| Angle | Intensity | Angle | Intensity | Angle | Intensity |
|---|---|---|---|---|---|
| °2-Theta | % | °2-Theta | % | °2-Theta | % |
| 7.748 | 92.8 | 15.814 | 14.0 | 25.995 | 15.4 |
| 8.380 | 17.8 | 16.496 | 43.9 | 26.753 | 18.4 |
| 8.950 | 14.3 | 17.349 | 40.9 | 28.085 | 20.4 |
| 9.515 | 8.9 | 17.687 | 100.0 | 28.883 | 19.9 |
| 10.043 | 11.7 | 18.229 | 35.3 | 29.737 | 19.7 |
| 11.000 | 13.2 | 18.736 | 40.9 | 30.227 | 21.6 |
| 11.829 | 17.5 | 19.754 | 37.5 | 31.204 | 28.1 |
| 12.258 | 17.3 | 20.258 | 50.3 | 31.729 | 20.6 |
| 13.087 | 19.0 | 20.498 | 93.1 | 34.825 | 19.7 |
| 13.464 | 22.3 | 21.719 | 47.4 | 35.394 | 24.2 |
| 13.808 | 13.4 | 22.499 | 35.5 | 36.018 | 22.9 |
| 14.840 | 14.3 | 23.310 | 29.9 | 37.722 | 24.5 |
| 15.197 | 27.1 | 24.111 | 19.5 | 39.304 | 20.6 |
| 15.489 | 21.4 | 25.165 | 19.1 | | |

b) Higher Melting Form

The title product from Example 29 was dissolved in methanol (450 mg in 15 ml-33 ml/g). Sodium hydroxide (1.33 mls as 1N solution) was added. The methanol was stripped using a rotavap in vacuo to give a gum. The gum was azeotroped once with isopropyl alcohol (8 mls) to remove residual methanol and water before a further portion of isopropyl alcohol (15 mls) was added. The mixture was heated until a homogenous mixture was obtained. On cooling crystallisation of the sodium salt occurred. The mixture was held at room temperature for 10 minutes. The salt was filtered off, washed with isopropyl alcohol and dried in a vacuum oven at 60° C. for 30 min; 200 mg of sodium salt was recovered; m.p. 252 degC (determined using a TA instruments DSC2910 at a heating rate of 10° C./minute).

The main peaks (from 2 to 40 degrees 20) in the PXRD pattern are as follows. The PXRD was performed without reference to an internal standard (e.g. silicon powder). The peak positions are therefore subject to possible instrumental zero offset and sample height errors.

| Angle | Intensity | Angle | Intensity | Angle | Intensity |
|---|---|---|---|---|---|
| °2-Theta | % | °2-Theta | % | °2-Theta | % |
| 3.419 | 0.8 | 20.682 | 1.5 | 30.090 | 1.1 |
| 6.674 | 100.0 | 20.758 | 1.7 | 30.735 | 0.9 |
| 7.394 | 0.4 | 20.841 | 1.4 | 31.360 | 1.4 |
| 8.200 | 1.7 | 21.058 | 1.0 | 32.025 | 3.5 |
| 11.465 | 0.4 | 21.462 | 0.5 | 32.742 | 0.7 |
| 13.010 | 1.5 | 21.935 | 2.0 | 33.072 | 0.7 |
| 13.337 | 0.8 | 22.965 | 0.6 | 33.723 | 1.2 |
| 16.414 | 0.7 | 23.585 | 1.1 | 34.261 | 0.7 |
| 17.288 | 2.8 | 24.868 | 1.4 | 35.946 | 1.8 |
| 18.165 | 0.9 | 25.387 | 1.1 | 36.839 | 0.7 |
| 18.987 | 1.4 | 25.733 | 1.8 | 37.484 | 0.7 |
| 19.330 | 0.5 | 26.124 | 0.9 | 38.519 | 1.4 |
| 19.687 | 0.8 | 26.826 | 0.9 | 38.801 | 1.3 |
| 20.069 | 1.8 | 28.719 | 1.8 | | |

The title compound of Example 29 metabolysed to form (2R)-1-(2-{[(5-ethyl-1,3,4-thiadiazol-2-yl)amino]carbonyl}pentyl)cyclopentanecarboxylic acid.

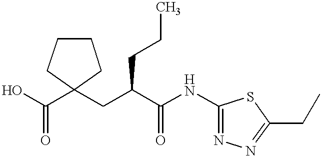

This compound was prepared as follows:

The product from Preparation 102 (430 mg, 1 mmol) was taken up in ethanol (5 mls) and methanol (1 ml) and hydrogenated at 30 psi hydrogen pressure at room temperature for 2 h. The mixture was then filtered through a plug of Arbocel®) and evaporated to a yellow oil. This oil was purified by column chromatography using firstly 19:1, then 9:1 DCM:MeOH as eluant to provide the product as a clear oil (120 mg, 35%); ¹HNMR (400 MHz, CDCl₃) 0.88 (t, 3H), 1.20-1.88 (m, 13H), 1.90-2.03 (m, 1H), 2.24-2.38 (m, 1H), 2.43-2.72 (m, 2H), 2.95 (q, 2H); LRMS m/z 340.2 (M+H).

### Example 31

### (R)-2-{[1-({[2-(Hydroxymethyl)-2,3-dihydro-1H-inden-2-yl]amino}carbonyl)-cyclopentyl]methyl}pentanoic acid

and

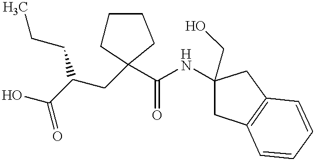

### Example 32

### (S)-2-{[1-({[2-(Hydroxymethyl)-2.3-dihydro-1H-inden-2-yl]amino}carbonyl)-cyclopentyl]methyl}pentanoic acid

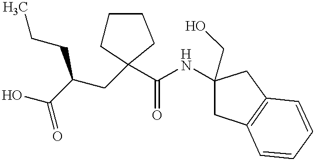

2-{[1-({[2-(Hydroxymethyl)-2,3-dihydro-1H-inden-2-yl]amino}carbonyl)-cyclopentyl]methyl}pentanoic acid (WO 9110644, Example 8) was further purified by HPLC using an AD column and hexane:isopropanol:trifluoroacetic acid (90:10:0.1) as eluant, to give the title compound of Example 31, 99% ee, [α]_{D}=+10.40 (c=0.067, ethanol) and the title compound of Example 32, 99% ee, [α]_{D}=−10.9° (c=0.046, ethanol).

### Example 33

### (2R)-2-[(1-{[(1-Benzyl-6-oxo-1,6-dihydro-3-pyridinyl)amino]carbonyl}-cyclopentyl)methyl]-pentanoic acid

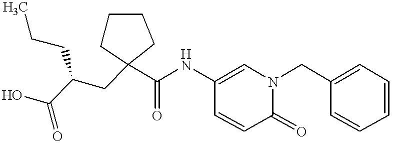

1-(3-Dimethylaminopropyl)-3-ethylcarbodiimide hydrochloride (191 mg, 1.0 mmol), 1-hydroxybenzotriazole hydrate (135 mg, 01.0 mmol), N-methylmorpholine (165 μl, 1.5 mmol) and finally the amine from preparation 28 (150 mg, 0.69 mmol) were added to a solution of the acid from preparation 2 (284 mg, 1.0 mmol) in N,N-dimethylformamide (8 ml), and the reaction stirred at 90° C. for 18 hours. The cooled solution was diluted with ethyl acetate (90 ml), washed with water (4×50 ml) and brine (50 ml), then dried (MgSO₄) and evaporated under reduced pressure. The crude product was purified by chromatography on silica gel, using ethyl acetate:pentane (30:70) to give a yellow oil, 191 mg. This intermediate was dissolved in dichloromethane (3 ml) and trifluoroacetic acid (3 ml) and the solution stirred at room temperature for 5 hours. The mixture was concentrated under reduced pressure and the residue purified by column chromatography on silica gel using dichloromethane:methanol (95:5) as eluant to give the title compound as a foam, 77 mg, ¹H NMR (CDCl₃, 300 MHz) δ: 0.86 (t, 3H), 1.20-1.76 (m, 12H), 1.93-2.02 (m, 1H), 2.20-2.46 (m, 3H), 4.95 (d, 1H), 5.04 (d, 1H), 6.61 (d, 1H), 7.21 (m, 1H), 7.50 (s, 1H), 8.23 (s, 1H); LRMS: m/z 411.6 (MH)⁺; [α]_{D}=−3.80 (c=0.052, ethanol).

### Example 34

### (2R)-2-[(1-{[(4-Butyl-2-pyridinyl)amino]carbonyl}cyclopentyl)methyl]pentanoic acid

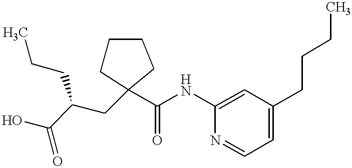

The title compound was obtained in 43% yield from the acid from preparation 2 and the amine from preparation 30, following a similar procedure to that described in Example 33, ¹H NMR (CDCl₃, 400 MHz) δ: 0.80-1.00 (m, 6H), 1.22-1.84 (m, 18H), 2.03-2.56 (m, 3H), 2.77 (m, 1H), 7.14 (d, 1H), 8.08 (d, 1H), 8.23 (s, 1H), 11.71 (brs, 1H); LRMS: m/z 361.7 (MH)⁺, [α]_{D}=−1.40 (c=0.14, ethanol).

### Example 35

### 2-[(1-{[(1-Benzyl-6-oxo-1,6-dihydro-3-pyridinyl)amino]carbonyl}cyclopentyl)methyl]-4-methoxybutanoic acid

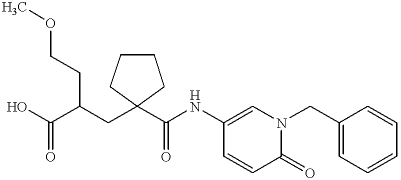

A mixture of the benzyl ester from preparation 62 (850 mg, 1.64 mmol), and 5% palladium on charcoal (250 mg) in 40% aqueous ethanol (21 ml), was hydrogenated at 30 psi and room temperature for 30 minutes. The reaction mixture was filtered through Hyflo®, and the filtrate evaporated under reduced pressure. The residual foam was purified by column chromatography on silica gel using dichloromethane:methanol (97:3) as eluant to give the title compound as a white foam, 550 mg, 79%; ¹H NMR (DMSO-d₆, 300 MHz) δ: 1.24-2.17 (m, 12H), 2.18-2.31 (m, 1H), 3.07 (s, 3H), 3.21 (t, 2H), 5.08 (s, 2H), 6.63 (d, 1H), 7.23-7.41 (m, 5H), 7.72 (d, 1H), 8.24 (s, 1H);

Anal. Found: C, 67.46; H, 7.18; N, 6.24. C₂₄H₃₀N₂O₅ requires C, 67.58; H, 7.09; N, 6.57%.

### Example 36

### 3-{1-[(Cyclopentylamino)carbonyl]cyclopentyl}-2-[(2-methoxyethoxy)methyl]-propanoic acid

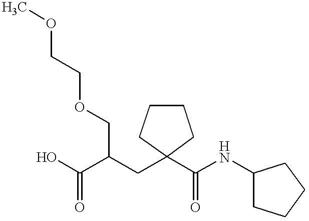

A solution of the tert-butyl ester from preparation 64 (320 mg, 0.80 mmol) in trifluoroacetic acid (2 ml) and dichloromethane (2 ml) was stirred at room temperature for 8 hours. The mixture was concentrated under reduced pressure and the residue azeotroped twice with toluene. The crude product was purified by column chromatography on silica gel using dichloromethane:methanol (95:5) to give the title compound as a clear oil, 171 mg, 62%; ¹H NMR (CDCl₃, 400 MHz) δ: 1.29-1.40 (m, 2H), 1.42-1.69 (m, 10H), 1.75 (dd, 1H), 1.87-2.03 (m, 5H), 2.64 (m, 1H), 3.34 (s, 3H), 3.43-3.52 (m, 3H), 3.57 (m, 2H), 3.61 (m, 1H), 4.08-4.20 (m, 1H), 5.89 (d, 1H); LRMS: m/z 340 (MH⁻).

### Example 37

### 3-(2-Methoxyethoxy)-2-{[1-({[3-(2-oxo-1-pyrrolidinyl)propyl]amino}carbonyl)-cyclopentyl]methyl}propanoic acid

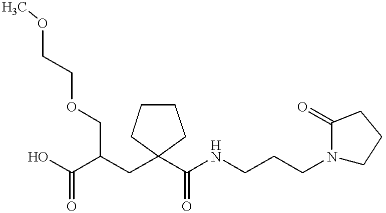

The title compound was obtained as a clear oil in 57% yield from the tert-butyl ester of preparation 65, following the procedure described in example 36, ¹H NMR (CDCl₃, 300 MHz) δ: 1.56-1.78 (m, 8H), 1.94-2.17 (m, 6H), 2.44 (m, 2H), 2.68-2.76 (m, 1H), 3.10-3.21 (m, 1H), 3.22-3.31 (m, 1H), 3.37 (s, 3H), 3.40 (m, 2H), 3.44-3.56 (m, 5H), 3.60 (m, 2H), 3.68 (m, 1H), 6.91-7.01 (m, 1H); LRMS: m/z 398.7 (M⁺).

### Example 38

### cis-3-(2-Methoxyethoxy)-2-[(1-{[(4-{[(phenylsulfonyl)amino]carbonyl}cyclohexyl)-amino]carbonyl}cyclopentyl)methyl]propanoic acid

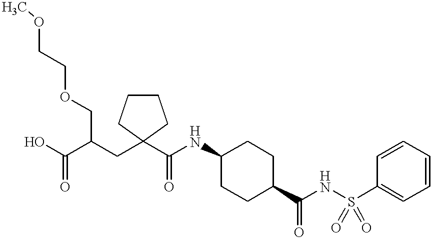

A solution of the tert-butyl ester from preparation 66 (446 mg, 0.75 mmol) in dichloromethane (5 ml) and trifluoroacetic acid (5 ml) was stirred at room temperature for 18 hours. The reaction mixture was concentrated under reduced pressure, and the residue azeotroped with dichloromethane, then toluene, and finally ether, to afford the title compound as a white foam, 385 mg, 95%; ¹H NMR (CDCl₃, 400 MHz) δ: 1.48-2.17 (m, 18H), 2.40 (s, 1H), 2.66 (s, 1H), 3.37 (s, 3H), 3.50-3.70 (m, 6H), 3.94 (s, 1H), 6.10 (d, 1H), 6.59 (s, 1H), 7.55 (t, 2H), 7.61 (m, 1H), 8.02 (d, 2H), 9.11 (s, 1H);

Anal. Found: C, 54.88; H, 6.90; N, 5.04. C₂₆H₃₈N₂O₈S;1.7H₂O requires C, 57.97; H, 7.11; N, 5.20%.

### Example 39

### 2-{[1-({[3-(Methylamino)-3-oxopropyl]amino}carbonyl)cyclopentyl]methyl}-4-phenylbutanoic acid

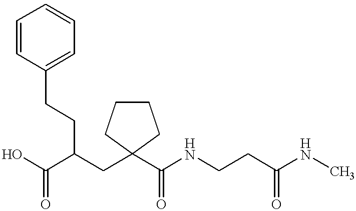

A mixture of the benzyl ester from preparation 68 (160 mg, 0.34 mmol) and 10% palladium on charcoal (100 mg) in ethanol (30 ml) was hydrogenated at room temperature and 60 psi for 18 hours. The mixture was filtered through Arbocel® and the filtrate concentrated under reduced pressure, and azeotroped with dichloromethane. The crude product was purified by column chromatography on silica gel using an elution gradient of dichloromethane:methanol:acetic acid (95:5:0 to 95:5:0.5) to afford the title compound as a white foam, 100 mg, 79%; ¹H NMR (CDCl₃, 400 MHz) δ: 1.40-1.70 (m, 8H), 1.95 (m, 3H), 2.10 (m, 1H), 2.35 (d, 3H), 2.59 (m, 2H), 2.75 (t, 3H), 3.42 (m, 2H), 6.25 (bs, 1H), 6.70 (bs, 1H), 7.13-7.25 (m, 5H); and LRMS: m/z 375.0 (MH⁺).

### Example 40

### 2-{[1-({[3-(2-Oxo-1-pyrrolidinyl)propyl]amino}carbonylcyclopentyl]-methyl}-4-phenylbutanoic acid

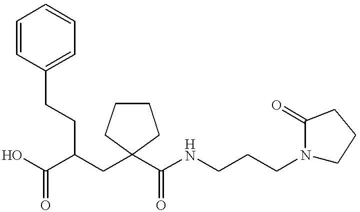

A mixture of the benzyl ester from preparation 67 (780 mg, 1.55 mmol) and 10% palladium on charcoal (100 mg) in ethanol:water (90:10 by volume; 30 ml) was hydrogenated at room temperature under 60 psi H₂ pressure for 1.5 hours. The catalyst was filtered off, and the filtrate evaporated under reduced pressure to provide the title compound as a white foam, 473 mg, 74%;

¹H NMR (CDCl₃, 300 MHz) δ: 1.26-1.77 (m, 10H), 1.78-2.46 (m, 1H), 2.49-2.70 (m, 2H), 2.95-3.36 (m, 4H), 6.92-7.38 (m, 5H);

Anal. Found: C, 64.05; H, 7.73; N, 6.22. C₂₄H₃₄N₂O₄; 0.75H₂O requires C, 65.88; H, 7.83; N, 6.40%.

### Example 41

### 4-Phenyl-2-({1-[(3-pyridinylamino)carbonyl]cyclopentyl}methyl)butanoic acid

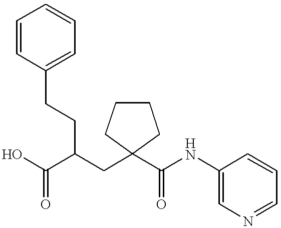

A mixture of the benzyl ester from preparation 71 (700 mg, 1.53 mmol) and 5% palladium on charcoal (70 mg) in ethanol:water (90:10 by volume, 50 ml) was hydrogenated at room temperature under 30 psi H₂ pressure for 5 hours. The catalyst was filtered through Arbocel®, washing well with ethanol, and the filtrate evaporated under reduced pressure. The crude product was purified by column chromatography on silica gel using dichloromethane:methanol (95:5) as the eluant to provide the title compound as a white foam, 510 mg, 91%; m.p. 80-85° C. (collapses to a gum); ¹H NMR (CDCl₃, 300 MHz) δ: 1.40-2.78 (m, 15H), 6.93-7.39 (m, 5H), 7.93 (m, 1H), 8.59 (d, 1H), 9.17 (d, 1H), 9.41 (s, 1H);

Anal. Found: C, 70.83; H, 7.10; N, 7.64. C₂₂H₂₆N₂O₃; 0.3H₂O requires C, 70.94; H, 7.22; N, 7.52%.

### Example 42

### 2-{[1-({[1-(Hydroxymethyl)cyclopentyl]amino}carbonyl)cyclopentyl]methyl}-4-phenylbutanoic acid

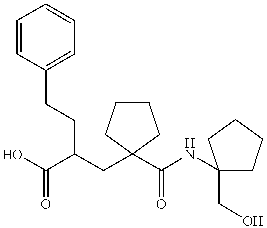

A mixture of the benzyl ester from preparation 69 (118 mg, 0.25 mmol) and 10% palladium on charcoal (100 mg) in ethanol (20 ml) was hydrogenated at room temperature and 60 psi for 18 hours. The mixture was filtered through Arbocel®, the filtrate concentrated under reduced pressure, and azeotroped with dichloromethane to give the title compound as a colourless gum, 95 mg, 98%; ¹H NMR (CDCl₃, 300 MHz) δ: 1.41-1.80 (m, 17H), 1.90 (m, 1H), 1.92-2.20 (m, 3H), 2.40 (m, 1H), 2.60 (m, 2H), 3.60 (d, 1H), 3.71 (d, 1H), 5.80 (bs, 1H), 7.15-7.30 (m, 5H); LRMS: m/z 388.1 (MH⁺).

### Example 43

### 2-[(1-{[(5-Methyl-1,3,4-thiadiazol-2-yl)amino]carbonyl}cyclopentyl)methyl]-4-phenylbutanoic acid

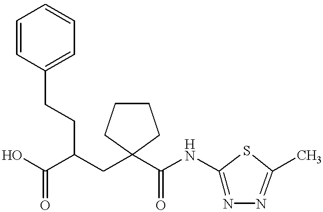

A mixture of the benzyl ester from preparation 70 (187 mg, 0.39 mmol) and 10% palladium on charcoal (80 mg) in ethanol (20 ml) was hydrogenated at 60 psi for 18 hours. Tlc analysis showed starting material remaining, so additional 10% palladium on charcoal (100 mg) was added, and the reaction continued for a further 5 hours. Tic analysis again showed starting material remaining, so additional catalyst (100 mg) was added, and hydrogenation continued for 18 hours. The mixture was filtered through Arbocel®, and the filtrate concentrated under reduced pressure, and azeotroped with dichloromethane. The crude product was purified by chromatography on silica gel using a Biotage® column, and dichloromethane:methanol (95:5) as eluant to afford the title compound as a clear oil, 80 mg, 53%; ¹H NMR (CDCl₃, 300 MHz) δ: 1.51-1.89 (m, 9H), 2.03 (m, 1H), 2.20 (m, 1H), 2.40 (m, 2H), 2.60 (m, 5H), 7.15-7.30 (m, 5H); LRMS: m/z 387.8 (MH⁺).

### Example 44

### (R)-2-{[1-({[2-(Hydroxymethyl)-2,3-dihydro-1H-inden-2-yl]amino}carbonyl)-cyclopentyl]-methyl}-4-phenylbutanoic acid

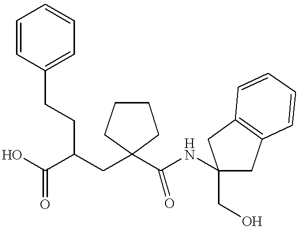

and

### Example 45

### (S)-2-{[1-({[2-(Hydroxymethyl)-2,3-dihydro-1H-inden-2-yl]amino}carbonyl)-cyclopentyl]methyl}-4-Phenylbutanoic acid

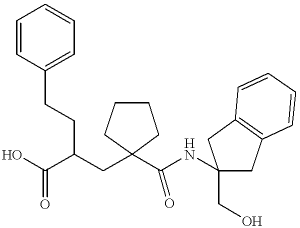

2-{[1-({[2-(Hydroxymethyl)-2,3-dihydro-1H-inden-2-yl]amino}carbonyl)-cyclopentyl]methyl}-4-phenylbutanoic acid (WO 9110644, Example 9) was purified by standard HPLC procedures using an AD column and hexane:isopropanol: trifluoroacetic acid (70:30:0.2) as eluant, to give the title compound of Example 44, 99.5% ee; [α][_{D}=+9.1° (c=1.76 in ethanol); and the title compound of Example 45, 99.5% ee; [α]_{D}=−10.50 (c=2.2 in ethanol).

### Example 46

### trans-3-[1-({[2-(4-Chlorophenyl)cyclopropyl]amino}carbonyl)cyclopentyl]-2-(methoxymethyl)propanoic acid

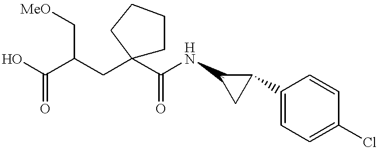

The product from preparation 72 (160 mg, 0.39 mmol) was taken up in 50 ml DCM and cooled to 0° C. Hydrogen chloride gas was then bubbled through the solution for 15 mins and then allowed to stir at room temperature for 16 h. The reaction mixture was concentrated in vacuo and then purified by column chromatography using 5:95 MeOH:DCM as eluant to provide the title product (18 mg, 12%); R_{f} 5:95 (DCM:MeOH) 0.2; ¹HNMR (400 MHz, CDCl₃) 1.04-1.18 (m, 2H), 1.20-1.36 (m, 2H), 1.36-1.79 (m, 7H), 1.83-2.08 (m, 4H), 2.57-2.66 (m, 1H), 2.73-2.83 (m, 1H), 3.27 (s, 3H, OMe), 3.32-3.41 (m, 1H), 3.48 (app. dd, 1H, CHOMe), 6.21 (s, NH), 7.03 (d, 2H, Ar), 7.18 (d, 2H, Ar); LRMS: m/z, M−H 378; HRMS Found MH+380.1622. Calculated MH+380.1623.

### Example 47

### trans-3-[1-({[2-(4-Methoxyphenyl)cyclopropyl]amino}carbonyl)cyclopentyl]-2-(methoxyethyl)propanoic acid

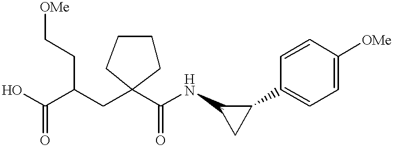

The product from preparation 81 (113 mg, 0.25 mmol) was taken up in a 4M solution of hydrogen chloride in dioxane (10 mls) and stirred for 3 h. The mixture was concentrated in vacuo and purified by column chromatography using 5:95 (MeOH:DCM) as eluant to provide the acid as a colourless film (45 mg, 44%); R_{f} 95:5 (DCM:MeOH) 0.2; LRMS: m/z, M−H, 388; ¹HNMR (400 MHz, CDCl₃) 1.01-1.22 (m, 2H), 1.40-2.22 (m, 15H), 2.42-2.57 (m, 1H), 2.73-2.82 (m, 1H), 3.23 (s, 3H, OMe), 3.27-3.44 (m, 2H), 3.72 (s, 3H, OMe), 6.12 (s, 1H, NH), 6.78 (d, 2H, Ar), 7.06 (d, 2H, Ar).

Compounds of formula Id, i.e. compounds of formula I where R¹ is methoxyethyl were prepared either from a) the indicated tert-butyl ester following a similar procedure to that described in Example 47, or b) the indicated benzyl ester following a similar procedure to described in Example 42.

| | |
|---|---|
| | (Id) |
| 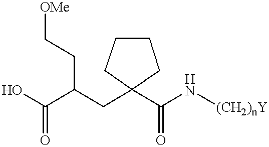 | |

| Ex | Prec | —(CH₂)ₙY | Data |
|---|---|---|---|
| 48 | Prep 89 | 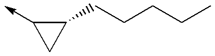 | ¹HNMR (CDCl₃, 400 MHz) δ: 0.50-0.63 (m, 3H), 0.82 (t, 3H, Me), 0.77-0.84 (m, 1H), 1.01-1.18 (m, 1H), 1.20-1.78 (m,), 1.82-2.08 (m, 2H), 3.27 (s, 3H, OMe), 3.33-3.41 (m, 2H), 5.92 (s, 1H, NH). LRMS: m/z 352 (M− H) |
| 49 | Prep 95 | 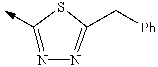 | ¹HNMR (CDCl₃, 400 MHz) δ: 0.62-2.57 (m, 13H), 3.07 (s, 3H, OMe), 2.96-3.44 (m, 2H), 4.09 (s, 2H), 7.20 (brs, 5H). HRMS: Found m/z 418.1796. C₂₁H₂₇N₃O₄S requires m/z 418.1795. |
| 50 | Prep 91 | 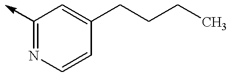 | ¹HNMR (CDCl₃, 400 MHz) δ: 0.92 (t, 3H, Me), 1.24-2.60 (m, 19H), 3.25 (s, 3H, OMe), 3.39 (t, 2H, CH₂OMe), 6.68-6.71 (m, 1H, Ar), 7.63-7.70 (m, 1H, Ar), 8.21 (s, 1H, Ar), 9.77 (brs, NH). LRMS: m/z 376 (M⁺). |
| 51 | Prep 92 | 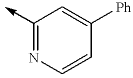 | ¹HNMR (CDCl₃, 400 MHz) δ: 1.24-2.34 (m, 10H), 2.37-2.54 (m, 1H), 2.54-2.73 (m, 2H), 3.33 (s, 3H, OMe), 3.38-4.49 (m, 2H), 7.00 (d, 1H. Ar), 7.38-7.56 (m, 3H), 7.59-7.69 (m, 2H), 7.80 (t, 1H, Ar), 8.66 (s, 1H, Ar), 9.77-9.93 (m, NH). LRMS: m/z 395 (M− H). |
| 52 | Prep 88 | 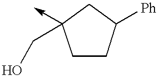 | ¹HNMR (CDCl₃, 400 MHz) δ: 0.84 (br.t, 3H), 1.20-2.20 (m, 19H), 2.24-2.58 (m, 2H), 3.07-3.33 (m, 1H), 3.60-3.96 (m, 2H), 5.82-5.98 (m, 1H), 7.14-7.36 |
| | | | (m, 5H). LRMS m/z M − H 400 |
| 53 | Prep 93 | 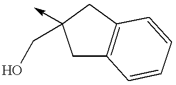 | ¹HNMR (CDCl₃, 400 MHz) δ: 1.43-1.76 (m, 7H), 1.80-2.24 (m, 4H), 2.57-2.68 (m, 2H), 3.06 (d, 1H), 3.12 (d, 1H), 3.27 (d, 1H), 3.32 (s, 3H), 3.36-3.48 (m, 2H), 3.80 (d, 1H), 3.87 (d, 1H), 6.04 (s, 1H), 7.16-7.22 (m, 4H). |
| 54 | Prep 81a | 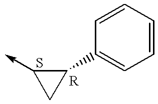 | ¹HNMR (CDCl₃, 400 MHz) δ: 1.02-1.26 (m, 2H), 1.37-1.84 (m, 7H), 1.85-2.16 (m, 4H), 2.62 (br.s, 1H), 2.80-2.93 (m, 1H), 3.29 (s, 3H, Me), 3.22-3.58 (m, 2H), 6.21 (br.s, 1H), 7.03-7.34 (m, 5H). LRMS m/z M + H 346 HRMS m/z M + H Found 346.2011. C₂₀H₂₇NO₄ requires 346.2013. |
| 55 | Prep 96 | 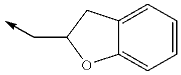 | ¹HNMR (CDCl₃, 400 MHz) δ: 1.45-1.67 (m, 8H), 1.85-2.00 (m, 4H), 2.05 (m, 1H), 2.46 (bs, 1H), 2.90 (dd, 1H), 3.30 (s, 3H), 3.35 (m, 2H), 3.40 (m, 2H), 3.70 (bm, 1H), 4.90 (bs, 1H), 6.25 (bs, 1H), 6.75 (d, 1H), 6.81 (t, 1H), 7.10 (d, 1H), 7.15 (t, 1H); |
| # LRMS m/z M− H 374; HRMS m/z M + H Found 376.2118. C₂₀H₂₇NO₄ requires 376.2123. | | | |

### Example 56

### (R)-2-[{1-({[2-(Hydroxymethyl)-2,3-dihydro-1H-inden-2-yl]amino}carbonyl)cyclopentyl]methyl}-4-methoxybutanoic acid

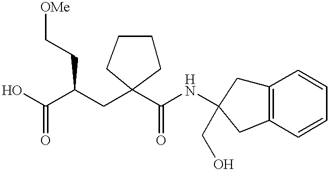

and

### Example 57

### (S)-2-{[1-({[2-(Hydroxymethyl)-2,3-dihydro-1H-inden-2-yl]amino}carbonyl)cyclopentyl]methyl}-4-methoxybutanoic acid

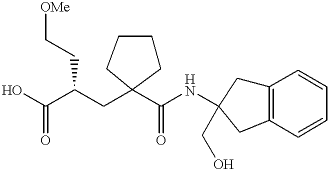

The product from Example 53 was purified by HPLC using a Chiralcel OD column (250*20 mm) at ambient temperature using a mixture of 70% hexane containing 0.3% TFA and 0.2% DEA and 30% IPA containing 0.3% TFA and 0.2% DEA at a flow rate of 10 ml/min. Example 55 is the R enantiomer which eluted first after 6 mins (α_{D} 11.00 c1 mg/ml in EtOH). Example 56 is the S enantiomer which eluted second after 7 mins (α_{D} −8.62 c1.07 mg/ml in EtOH).

### Example 58

### 3-Methoxy-2-[{1-({[(trans)-2-phenylcyclopropyl]amino}carbonyl)cyclopentyl]methyl}propanoic acid

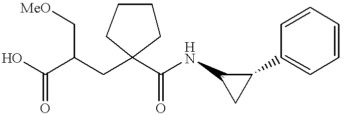

The title compound was prepared according to the procedure of Example 47 from the title product from preparation 82; ¹HNMR (CDCl₃, 400 MHz) δ: 1.2 (m, 2H), 1.5 (m, 3H), 1.6 (bs, 3H), 1.8 (d, 1H), 2.0 (m, 4H), 2.6 (bs, 1H), 2.9 (bs, 1H), 3.3 (s, 3H), 3.4 (t, 1H), 3.5 (t, 1H), 6.4 (s, 1H), 7.1 (m, 3H), 7.3 (t, 2H); LRMS 344 (M−H).

The following Preparations describe the preparation of certain intermediates used in the preceding Examples.

Preparation 1

1-[2-(tert-Butoxycarbonyl)-4-pentyl]-cyclopentane carboxylic acid

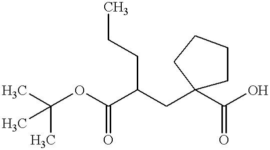

A mixture of 1-[2-(tert-butoxycarbonyl)-4-pentenyl]-cyclopentane carboxylic acid (EP 274234, Example 44) (23 g, 81.5 mmol) and 10% palladium on charcoal (2 g) in dry ethanol (200 ml) was hydrogenated at 30 psi and room temperature for 18 hours. The reaction mixture was filtered through Arbocel®, and the filtrate evaporated under reduced pressure to give a yellow oil. The crude product was purified by column chromatography on silica gel, using ethyl acetate:pentane (40:60) as the eluant, to provide the title product as a clear oil, 21 g, 91%; ¹H NMR (CDCl₃, 0.86 (t, 3H), 1.22-1.58 (m, 15H), 1.64 (m, 4H), 1.78 (dd, 1H), 2.00-2.18 (m, 3H), 2.24 (m, 1H); LRMS: m/z 283 (M−H)⁻.

Preparation 2

1-[(2R)-2-(tert-Butoxycarbonyl)-4-pentyl]-cyclopentane carboxylic acid

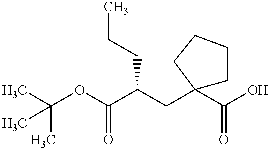

A mixture of (R)-1-[2-(tert-butoxycarbonyl)-4-pentenyl]-cyclopentane carboxylic acid (WO 9113054, Example 10) (10 g, 35.4 mmol) and 10% palladium on charcoal (600 mg) in dry ethanol (25 ml) was hydrogenated at 1 atm. and room temperature for 18 hours. The reaction mixture was filtered through Arbocel®, and the filtrate evaporated under reduced pressure to give the title compound as a yellow oil, 9.6 g, 95%; ¹H NMR (CDCl₃, 0.86 (t, 3H), 1.22-1.58 (m, 15H), 1.64 (m, 4H), 1.78 (dd, 1H), 2.00-2.18 (m, 3H), 2.24 (m, 1H); [α]_{D}=−3.3° (c=0.09, ethanol).

Alternatively the title product from Preparation 2 may be prepared as follows:

A solution of the product from stage f) below (1.12 kg, 4.0 mol) in ethanol (6.5 L) was split into two equal portions which were both subjected to the following reaction conditions. To a solution of the product from stage f) below (561 g, 2.0 mol) in ethanol (3.25 L) was added the hydrogenation catalyst (50.5 g of 5% Pd on carbon −50% wet) and the reaction vessel was pressurised with hydrogen gas (30 psi). The reaction was stirred for 18 hours at room temperature before recombining the two batches and removing the catalyst by filtration. The filter cake was washed with ethanol (2×450 ml) and the combined filtrates were then concentrated under vacuum. The resultant suspension was then filtered and n-heptane (1 L) was added. The solvent was removed by distillation under vacuum and n-heptane (1 L) was added. The solvent was removed by distillation under vacuum to give the title compound (1.1 kg, 3.86 mol, 97% yield) as a yellow oil that was used directly in the next step; LRMS (negative APCI): m/z [M−H]⁻ 283; ¹H-NMR (CDCl₃, 300 MHz) δ: 0.79-0.98 (t, 3H), 1.24-1.39 (m, 3H), 1.42-1.50 (s, 9H), 1.50-1.61, (m, 3H), 1.61-1.74 (m, 4H), 1.74-1.84 (m, 1H), 2.02-2.23 (m, 3H), 2.23-2.35 (m, 1H).

Preparation of Starting Materials

a) tert-Butyl-3-bromopropionate

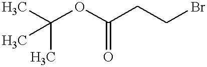

To a solution of 3-bromopropionic acid (6.0 kg, 39.2 mol) in dichloromethane (60 L) at 0° C. was added tert-butanol (0.6 L) and conc. sulfuric acid (0.33 L). The resultant solution was cooled to −15° C. and isobutylene was bubbled through (11 kg, 196 mol). The reaction was then stirred for 3 hours at −5° C. before warming to 20° C. over 4 hours and was then stirred at this temperature for 15 hours. The reaction was quenched by cautious addition into saturated aqueous sodium bicarbonate solution (0.6 M, 72 L, 43.2 mol). The layers were then separated and the organic layer was washed with saturated aqueous sodium bicarbonate solution (2×48 L) followed by deionised water (48 L). This washing cycle was repeated and the pH of the aqueous layer was measured and was shown to be above pH 7. Potassium carbonate (90 g, 1.5% w/w) was added to the organic layer before concentrating the solution to a volume of 9 L by distillation at atmospheric pressure. Tetrahydrofuran (40 L) was added and the remainder of the dichloromethane was removed by distillation at atmospheric pressure to give a solution (12 L) of the title compound (5.27 kg, 25.2 mol, 64% yield) in tetrahydrofuran that was used directly in the next step; ¹H-NMR (CDCl₃, 300 MHz,) δ: 1.45 (s, 9H), 2.80 (t, 2H), 3.53 (t, 2H); LRMS (El): m/z [MH⁺] 209 (⁷⁹Br).

b) 1-(3-tert-Butoxy-3-oxopropyl)cyclopentane carboxylic acid

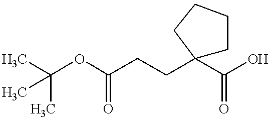

To a solution of commercially supplied lithium diisopropylamide (20.2 kg of a 2M solution in tetrahydrofuran/n-heptane/ethylbenzene, 51.0 mol) at −15° C. was added a solution of cyclopentane carboxylic acid (2.7 kg, 23.7 mol) in anhydrous tetrahydrofuran (8.1 L) cautiously over a period of 1 hour with stirring under a nitrogen atmosphere. The resultant solution was stirred at 0° C. for 3 hours during which time a precipitate formed. This suspension was then added to a solution of product from stage a) above (5.24 kg, 25.1 mol) in tetrahydrofuran (52 L) at −15° C. over a period of 1.25 hours. After the addition was complete, the reaction was stirred at 0° C. for 1 hour and then warmed to 20° C. over 4 hours and left to stir at this temperature for 13.5 hours. The reaction mixture was then cooled to −15° C. and to this was added n-heptane (27 L) and 5M aqueous hydrochloric acid (23.7 L, 118.5 mol) cautiously with stirring. The layers were then separated and the aqueous phase was extracted with n-heptane (13.5 L). The combined organic phases were extracted with 5% aqueous sodium bicarbonate solution (3×30 L) and then with 10% aqueous potassium carbonate solution (3×30 L). The aqueous extracts were kept separate and analysed for product content. The three aqueous potassium carbonate extracts were combined and n-heptane (27 L) was added before the pH of this mixture was then adjusted to pH 7-8 using 5 M aqueous hydrochloric acid (10.5 L) with stirring. The layers were then separated and more n-heptane (40.5 L) was added. The pH of the mixture was then adjusted further to pH 3 using 5 M aqueous hydrochloric acid (19.5 L). The layers were then separated and the organic phase was washed with deionised water (2×27 L). The organic phase then azeotropically dried by distillation under vacuum and the solvent volume was reduced to approximately 4 L. The solution was then cooled to 0° C. with stirring to allow crystallisation to occur. Stirring was continued at 0° C. for 5 hours after which the product was collected by filtration. The resultant solid was dried under vacuum at 50° C. for 22.5 hours to give the title compound (1.17 kg, 4.8 mol, 20% yield) as a white crystalline solid; m.p.: 89-92° C.; LRMS (negative APCl): m/z [M−H]⁻ 241; ¹H-NMR (CDCl₃, 300 MHz) δ: 1.47 (s, 9H), 1.50-1.60 (m, 2H), 1.60-1.82 (m, 4H), 1.69-2.0 (m, 2H), 2.08-2.23 (m, 2H), 2.23-2.34 (m, 2H).

c) 1-[2-(tert-Butoxycarbonyl)-4-pentenyl]cyclopentane carboxylic acid

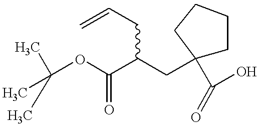

To a solution of commercially supplied lithium diisopropylamide (7.63 kg of a 2M solution in tetrahydrofuran/n-heptane/ethylbenzene, 19.3 mol) in anhydrous tetrahydrofuran (18.3 L) at −10° C. was added a solution of the product from stage b) above (2.0 kg, 8.25 mol) in anhydrous tetrahydrofuran (10 L) with stirring over a period of 4 hours whilst maintaining the reaction temperature at −10° C. To the resultant solution was added a solution of allyl bromide (1.2 kg, 9.9 mol) in tetrahydrofuran (10 L) over a period of 2 hours before warming the reaction to 20° C. over a period of 4 hours. After stirring at this temperature for 9.5 hours the reaction was quenched by the addition of water (40 L) and the two phases were separated. The organic phase was then extracted successively with water (20 L) and 0.3 M aqueous potassium hydroxide solution (12 L). To the combined aqueous phases was then added n-heptane (20 L), and 5 M aqueous hydrochloric acid (7.5 L) until the pH of the aqueous layer was pH 2. The layers were then separated and the aqueous phases were then extracted with n-heptane (20 L). The combined organic phases were then washed with saturated brine (2×8.0 L) and concentrated under vacuum to give the crude product (2.22 kg, 7.86 mol, 95% yield) as a solution in n-heptane (12.5 kg total solution weight) that was used directly in the next step; LRMS (El): [M⁺−C₄H₈] 226, [M⁺—HO^{t}Bu] 208, [208-CO] 180; ¹H-NMR (CDCl₃, 300 MHz), δ: 1.45 (s, 9H), 1.48-1.60 (m, 2H), 1.60-1.76 (m, 4H), 1.80 (dd, 1H), 2.03-2.27 (m, 4H), 2.27-2.45 (m, 2H), 5.06 (dd, 2H), 5.75 (ddt, 1H).

d) Cyclohexanaminium 1-[2-(tert-butoxycarbonyl)-4-pentenyl]cyclopentane carboxylate

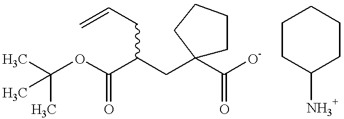

To a solution of crude product from stage c) above (3.83 kg, 13.6 mol) in n-heptane (23 L) was added cyclohexylamine (1.35 kg, 13.8 mol) in n-heptane (7.0 L) over a period of 0.5 hours. The delivery lines were washed with n-heptane (0.7 L, 0.2 mug) and this was added to the reaction mixture. The resultant slurry was granulated at 20° C. for 2 hours and the solid was then collected by filtration. The filter cake was washed with n-heptane (2×1.9 L) and was dried under vacuum at 50° C. for 23 hours. The resultant white solid (4.42 kg, 11.6 mol, 85% yield) was dissolved in ethyl acetate (24 L, 5.4 ml/g) and was heated to 70° C. to form a clear solution. The resultant solution was then cooled to 50° C. and was seeded with authentic compound (1 g). The suspension was then cooled from 50° C. to 20° C. over a period of 4 hours. The suspension was granulated at 20° C. for 0.5 hours and the solid was collected by filtration. The filter cake was washed with ethyl acetate (2×1.8 L), and the solid was dried under vacuum at 45° C. for 14.5 hours to give the title compound (3.76 kg, 9.85 mol, 85% recovery) as a white crystalline solid; m.p.: 129.5-131.0° C.;

Anal. Found: C, 69.28; H, 10.31; N, 3.60. C₂₂H₃₉NO₄ requires C, 69.25; H, 10.30; N, 3.67%; ¹H-NMR (CDCl₃, 300 MHz) δ: 1.05-1.35 (m, 6H), 1.35-1.53 (m, 10H), 1.53-1.69 (m, 5H), 1.69-1.89 (m, 3H), 1.89-2.02 (m, 3H), 2.02-2.18 (m, 2H), 2.18-2.31 (m, 2H), 2.31-2.44 (m, 1H), 2.71-2.94 (m, 1H), 5.03 (dd, 2H), 5.73 (ddt, 1H), 6.4 (bs, 3H).

e) (1S,2S)-1-Hydroxy-N-methyl-1-phenyl-2-propanaminium 1-[(2R)-2-(tert-butoxycarbonyl)-4-pentenyl]cyclopentane carboxylate

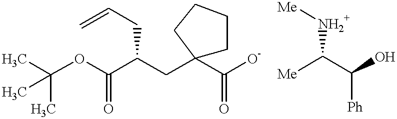

To a mixture of water (22.6 L) and n-heptane (22.6 L) was added the product from stage d) above (3.76 kg, 9.85 mol) with stirring. 5 M aqueous hydrochloric acid (2.2 L, 11.0 mol) was added until the pH of the aqueous phase was pH 3. The layers were separated and the aqueous phase was extracted further with n-heptane (2×22.6 L). The combined organic extracts were then washed with saturated brine (3.8 L, 10 ml/g) and were then concentrated under vacuum to a volume of 20.3 L total solution volume). To this solution was added (1S,2S)-(+)-pseudoephedrine (1.63 kg, 9.86 mol, 1.0 eq) with stirring and the suspension was heated to 80° C., whereupon complete dissolution occurred. The resultant solution was held at this temperature for 20 minutes before cooling to 45° C. A sample of seed crystals (0.2 g) was then added and the suspension was cooled to 20° C. over a period of 2 hours. The resultant slurry was granulated for a period of 4 hours and the solid was then collected by filtration. The filter cake was then washed with n-heptane (3×0.5 L) and was dried under vacuum at 40-45° C. for 23 hours to give a white solid (2.15 kg, 4.8 mol, 49% yield). A suspension of this material (2.15 kg, 4.8 mol) in n-heptane (10.8 L) was heated to 80° C. to give a clear solution. After holding this temperature for 10 minutes the solution was cooled to 60° C. and a sample of seed crystals (1 g) was added. The resultant suspension was then cooled to 20° C. over a period of 2 hours and was then granulated for 1.5 hours at this temperature. The solid was then collected by filtration, washed with n-heptane (2×0.59 L) and was dried under vacuum at 50° C. for 17.5 hours to give the title compound (1.80 kg, 4.0 mol, 84% recovery) as a white crystalline solid; m.p.: 109-110° C.;

Anal. Found: C, 69.48; H, 9.25; N, 3.17. C₂₆H₄₁NO₅ requires C, 69.77; H, 9.23; N, 3.17%; ¹H-NMR (300 MHz, CDCl₃) δ: 1.05 (d, 3H), 1.34-1.55 (m, 2H), 1.44 (s, 9H), 1.55-1.73 (m, 4H), 1.82-2.03 (m, 2H), 2.03-2.21 (m, 2H), 2.21-2.35 (m, 2H), 2.35-2.41 (m, 1H), 2.60 (s, 3H), 3.03 (pent, 1H), 4.52 (d, 1H), 5.03 (dd, 2H), 5.76 (ddt, 1H), 7.21-7.45 (m, 5H).

f) 1-[(2R)-2-(tert-Butoxycarbonyl)-4-pentenyl]cyclopentane carboxylic acid

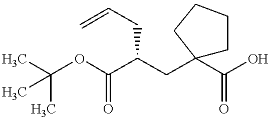

To a mixture of deionised water (10.8 L) and n-heptane (10.8 L) was added the product from stage e) above (1.80 kg, 4.0 mol) and 5 M aqueous hydrochloric acid (1.3 L, 6.5 mol) until the pH of the aqueous layer was pH 3. The layers were then separated and the aqueous layer was extracted with n-heptane (2×10.8 L). The combined organic layers were washed with brine (1.8 L) and were then concentrated by distillation at atmospheric pressure to a volume of 6.4 L. Ethanol (18.0 L) was then added and the solution was again concentrated by distillation at atmospheric pressure to give the title compound (1.14 kg, 4.0 mol, 100% recovery) as a solution in ethanol (6.4 L total solution volume) that was used directly in the next step (see above); LRMS (El): [M⁺—C₄H₈] 226, [M⁺—HO^{t}Bu] 208, [208-CO] 180; ¹H-NMR (300 MHz, CDCl₃)δ: 1.45 (s, 9H), 1.48-1.60 (m, 2H), 1.60-1.76 (m, 4H), 1.80 (dd, 1H), 2.03-2.27 (m, 4H), 2.27-2.45 (m, 2H), 5.06 (dd, 2H), 5.75 (ddt, 1H).

Preparation 3

Benzyl 2-{[1-(chlorocarbonyl)cyclopentyl]methyl}entanoate

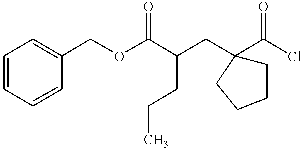

Oxalyl chloride (1.15 ml, 13.2 mmol) was added to an ice-cooled solution of 1-{2-[(benzyloxy)carbonyl]pentyl}cyclopentanecarboxylic acid (EP 274234, Example 16) (2.0 g, 6.3 mmol) in dry dichloromethane (20 ml), and the solution stirred at room temperature for 2 hours. The reaction mixture was concentrated under reduced pressure and the residue azeotroped with dichloromethane (3×), to give the title compound as a golden oil, 2.1 g; ¹H NMR (CDCl₃, 300 MHz) δ: 0.88 (t, 3H), 1.28 (m, 2H), 1.43 (m, 2H), 1.63 (m, 6H), 2.00 (m, 1H), 2.08-2.35 (m, 3H), 2.44 (m, 1H), 5.15 (s, 2H), 7.28 (m, 5H).

Preparation 4

1-(2-{[tert-Butyl(dimethyl)silyl]oxy}ethyl)-2-piperidinone

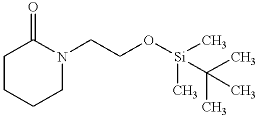

Sodium hydride (807 mg, 60% dispersion in mineral oil, 20.18 mmol) was added portionwise to a solution of d-valerolactam (2.0 g, 20.2 mmol) in tetrahydrofuran (100 ml) under nitrogen. (2-Bromoethoxy)(tert-butyl)dimethylsilane (ex Aldrich Chemical Co.) (4.33 ml, 20.2 mmol) was added portionwise, and the reaction heated at 70° C. for 18 hours. Water (50 ml) was added to the cooled reaction, the mixture concentrated in vacuo, to remove the tetrahydrofuran, and extracted with ethyl acetate (200 ml). The organic solution was dried (MgSO₄), and evaporated under reduced pressure to give a yellow oil. The crude product was purified by column chromatography on silica gel using an elution gradient of dichloromethane:methanol (98:2 to 97:3) to give the title compound, 3.25 g; ¹H NMR (CDCl₃, 400 MHz) δ: 0.00 (s, 6H), 0.83 (s, 9H), 1.75 (m, 4H), 2.35 (m, 2H), 3.39 (m, 4H), 3.75 (t, 2H); LRMS: m/z 257.9 (M⁺).

Preparation 5

1-(2-Hydroxyethyl)-2-piperidinone

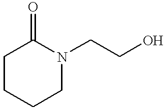

Tetra-n-butylammonium fluoride (14 ml,1M solution in tetrahydrofuran, 14 mmol) was added to a solution of the lactam from preparation 4 (3.3 g, 12.8 mmol) in tetrahydrofuran (50 ml), and the reaction stirred at room temperature for 2 hours. The mixture was concentrated under reduced pressure, the residue azeotroped with dichloromethane, and purified by column chromatography on silica gel using an elution gradient of dichloromethane:methanol (97:3 to 95:5) to give the title compound as an oil; ¹H NMR (CDCl₃, 400 MHz) δ: 1.80 (m, 4H), 2.40 (t, 2H), 3.38 (t, 2H), 3.42 (t, 1H), 3.56 (t, 2H), 3.80 (t, 2H).

Preparation 6

2-[2-(2-Oxo-1-piperidinyl)ethyl]-1H-isoindole-1,3(2H)-dione

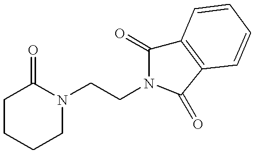

Pthalimide (952 mg, 6.47 mmol) was added to a solution of the product from preparation 5 (842 mg, 5.88 mmol) in tetrahydrofuran (30 ml), and the mixture sonicated until a solution was obtained. Polymer supported triphenyl phosphine (2.5 g, 7.5 mmol) and diethyl azodicarboxylate (1.15 ml, 7.31 mmol) were added, and the reaction stirred at room temperature for 18 hours. The mixture was filtered through Arbocel®, the filtrate concentrated under reduced pressure and the residue azeotroped with dichloromethane. The crude product was purified by column chromatography on silica gel using an elution gradient of ethyl acetate:pentane (70:30 to 100:0), to give the title compound as a white foam, 1.6 g (containing some impurities); ¹H NMR (CDCl₃, 400 MHz) δ: 1.60-1.80 (m, 4H), 2.17 (m, 2H), 3.30 (m, 2H), 3.60 (m, 2H), 3.83 (m, 2H), 7.62 (m, 2H), 7.79 (m, 2H); LRMS: m/z 273.2 (MH⁺).

Preparation 7

(1S,3R)-3-Aminocyclopentanecarboxylic acid

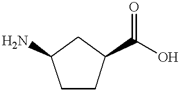

Platinum oxide (1 g) was added to a solution of (1R,4S)-4-amino-cyclopent-2-ene carboxylic acid (Taylor et al., J. Chem. Soc., Chem. Commun. (1990), (16), 1120-1) (5.3 g, 41.7 mmol) in water (70 ml), and the mixture was hydrogenated at 45 psi and room temperature for 18 hours. The mixture was filtered through Arbocel®, the filtrate evaporated under reduced pressure, and the residue azeotroped with toluene, to afford the title compound as an off-white solid; ¹H NMR (D₂O, 400 MHz) δ: 1.70-1.92 (m, 3H), 2.00 (m, 2H), 2.18 (m, 1H), 2.77 (m, 1H), 3.68 (m, 1H); LRMS: m/z 129.8 (MH⁺).

Preparation 8

(1S,3R)-3-[(tert-Butoxycarbonyl)amino]cyclopentanecarboxylic acid

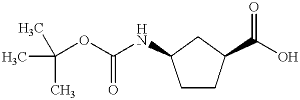

Di-tert-butyl dicarbonate (10 g, 45.8 mmol) was added to an ice-cooled solution of the product from preparation 7 (5.4 g, 41.8 mmol) in dioxan (42.5 ml) and sodium hydroxide solution (42.5 ml, 1N, 42.5 mmol), and the reaction stirred at room temperature for 18 hours. The reaction mixture was concentrated under reduced pressure to remove the dioxan, then acidifed to pH 2 using 2N hydrochloric acid. The aqueous solution was extracted with ethyl acetate (5×100 ml), the combined organic extracts dried (MgSO₄) and evaporated under reduced pressure to give a white solid. This was triturated with hexane, to give the title product, 8.0 g, 83%; ¹H NMR (CDCl₃, 400 MHz) δ: 1.41 (s, 9H), 1.58-2.06 (m, 5H), 2.21 (m, 1H), 2.84 (m, 1H), 4.01 (m, 1H), 4.84 (m,1H); LRMS: m/z 228 (M−H)⁻.

Preparation 9

3-[(tert-Butoxycarbonyl)amino]cyclohexanecarboxylic acid

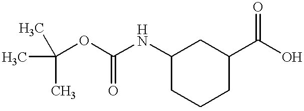

The title compound was obtained as a white solid in 81% yield, from 3-aminocyclohexanecarboxylic acid, following the procedure described in preparation 8; ¹H NMR (CDCl₃, 400 MHz) δ: 1.04 (m, 1H), 1.19-1.50 (m, 13H), 1.83 (m, 1H), 1.97 (m, 2H), 2.24 (m, 1H), 2.40 (m, 1H), 3.44 (bs, 1H), 4.42 (bs, 1H).

Preparation 10

tert-Butyl (1R,3S)-3-(aminocarbonyl)cyclopentylcarbamate

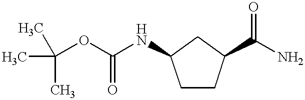

Benzotriazol-1-yloxytris(pyrrolidino)phosphonium hexafluorophosphate (3.4 g, 6.54 mmol), 1-hydroxybenzotriazole hydrate (883 mg, 6.54 mmol), ammonium chloride (467 mg, 8.72 mmol) and N-ethyldiisopropylamine (3.04 ml, 17.5 mmol) were added sequentially to a solution of the acid from preparation 8 (1.0 g, 4.37 mmol) in N,N-dimethylformamide (16 ml), and the reaction stirred at room temperature for 2 hours. The mixture was diluted with ethyl acetate (100 ml), washed with water (3×), and brine, then dried (MgSO₄) and evaporated under reduced pressure. The residual gum was purified by chromatography on silica gel using a Biotage(1 column, and an elution gradient of dichloromethane:methanol (98:2 to 95:5). The product was triturated with ether to afford the title compound as a white solid, 438 mg, 44%; ¹H NMR (DMSOd₆, 400 MHz) δ: 1.34 (s, 9H), 1.40 (m, 2H), 1.64 (m, 3H), 1.90 (m, 1H), 2.55 (m, 1H), 3.70 (m, 1H), 6.70 (bs, 1H), 6.80 (d, 1H), 7.22 (bs, 1H).

Preparation 11

tert-Butyl 3-[(dimethylamino)carbonyl]cyclohexylcarbamate

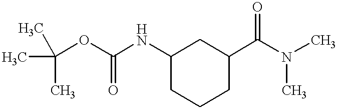

1-(3-Dimethylaminopropyl)-3-ethylcarbodiimide hydrochloride (1.19 g, 6.19 mmol), 1-hydroxybenzotriazole hydrate (840 mg, 6.19 mmol), N-methylmorpholine (1.1 ml, 10.1 mmol) and finally 33% ethanolic dimethylamine (1.5 ml) were added to a solution of the acid from preparation 9 (1.37 g, 5.6 mmol) in N,N-dimethylformamide (30 ml), and the reaction stirred at room temperature for 18 hours. The mixture was concentrated under reduced pressure, the residue diluted with ethyl acetate and washed with water (2×). The mixture was dried (MgSO₄) and evaporated under reduced pressure. The crude product was purified by column chromatography on silica gel using an elution gradient of methanol:dichloromethane (5:95 to 10:90), to give the title compound, 998 mg, 66%; ¹H NMR (CDCl₃, 300 MHz) δ: 1.12 (m, 1H), 1.40 (m, 11H), 1.70 (m, 2H), 1.85 (m, 1H), 2.00 (m, 2H), 2.62 (m, 1H), 2.96 (s, 3H), 3.05 (s, 3H), 3.50 (m,1H), 4.50 (m, 1H).

Preparation 12

tert-Butyl 2-(2-acetylhydrazino)-2-oxoethylcarbamate

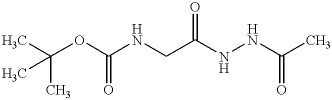

2-Ethoxy-1-ethoxycarbonyl-1,2-dihydroquinoline (7.06 g, 28.5 mmol) was added to a solution of N-tert-butoxycarbonylglycine (5.0 g, 28.6 mmol) in dichloromethane (75 ml), and the solution stirred for 15 minutes. Acetic hydrazide (2.6 g, 35.1 mmol) was added, and the reaction stirred at room temperature for 18 hours. The resulting precipitate was filtered, and dried in vacuo, to afford a white crystalline solid, 2.42 g. The filtrate was concentrated under reduced pressure, diluted with ether, and the resulting precipitate filtered and dried in vacuo, to afford additional product as a white solid, 4.4 g, 67% in total; ¹H NMR (CDCl₃, 400 MHz) δ: 1.41 (s, 9H), 2.02 (s, 3H), 3.87 (d, 2H), 5.22 (bs, 1H), 8.27 (bs, 1H), 8.84 (bs, 1H); LRMS: m/z 249.2 (MNH₄⁺);

Anal. Found: C, 46.41; H, 7.36; N, 17.98, C₉H17N₃O₄ requires C, 46.66; H, 7.41; N, 18.13%.

Preparation 13

Benzyl 3-(methylamino)-3-oxopropylcarbamate

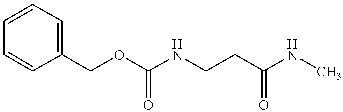

A mixture of N-[(benzyloxy)carbonyl]-β-alanine (10 g, 44.8 mmol), methylamine hydrochloride (3.33 g, 49.28 mmol), 1-hydroxybenzotriazole hydrate (6.05 g, 44.8 mmol), 1-(3-dimethylaminopropyl)-3-ethylcarbodiimide hydrochloride (1 0.3 g, 53.76 mmol) and N-methylmorpholine (11.33 ml, 103 mmol) in dichloromethane (200 ml) was stirred at room temperature for 18 hours. The resulting precipitate was filtered off to give the desired product as a colourless foam, and the filtrate evaporated under reduced pressure. The residue was purified by column chromatography on silica gel using an elution gradient of ethyl acetate:hexane (90:10 to 100:0) to give additional product, 7.96 g, 75% in total; ¹H NMR (CDCl₃, 300 MHz) δ: 2.42 (t, 2H), 2.80 (s, 3H), 3.50 (m, 2H), 5.21 (s, 2H), 5.49 (bs, 1H), 5.63 (bs, 1H), 7.36 (m, 5H);

Anal. Found: C, 60.68; H, 7.00; N, 11.95. C₁₂H₁₆N₂O₃ requires C, 61.00; H, 6.83; N, 11.86%.

Preparation 14

tert-Butyl (5-methyl-1 3,4-thiadiazol-2-yl)methylcarbamate

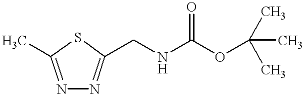

Lawesson's reagent (960 mg, 2.38 mmol) was added to a solution of the hydrazide from preparation 12 (500 mg, 2.16 mmol) in tetrahydrofuran (40 ml) and the reaction heated under reflux for 3 hours, then stirred at room temperature for 18 hours. The mixture was evaporated under reduced pressure and the residue purified by column chromatography on silica gel using an elution gradient of ethyl acetate:pentane (70:30 to 80:20) to give an oil. Ethyl acetate (100 ml) and charcoal (2 g) were added and the mixture was stirred for 10 minutes then filtered. The filtrate was concentrated under reduced pressure, and the residue azeotroped with dichloromethane to afford the title compound as a crystalline solid, 441 mg, 89%; ¹H NMR (CDCl₃, 400 MHz) δ: 1.45 (s, 9H), 2.77 (s, 3H), 4.66 (d, 2H), 5.22 (bs, 1H); LRMS: m/z 230.1 (MH⁺).

Preparation 15

N-Methoxy-N-methyl-2-(2-oxo-1-pyrrolidinyl)acetamide

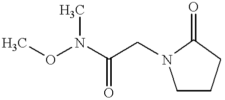

2-Chloro-N-methoxy-N-methylacetamide (ex Aldrich Chemical Co.) (3.2 g, 23.3 mmol) was added to a suspension of 2-pyrrolidinone (2.0 g, 23.5 mmol) and sodium hydride (940 mg, 60% dispersion in mineral oil, 23.5 mmol) in tetrahydrofuran (60 ml), and the reaction stirred at room temperature for 48 hours. The mixture was quenched with water (150 ml), and extracted with ethyl acetate (200 ml) and dichloromethane (200 ml). The combined organic extracts were dried (MgSO₄) and evaporated under reduced pressure. The residue was triturated with hexane, then ether to afford the title compound as white crystals, 1.8 g, 41%; ¹H NMR (CDCl₃, 400 MHz) δ: 2.02 (m, 2H), 2.40 (t, 2H), 3.17 (s, 3H), 3.48 (t, 2H), 3.72 (s, 3H), 4.19 (s, 2H); LRMS: m/z 186.9 (MH⁺).

Preparation 16

1-(2-Oxopropyl)-2-pyrrolidinone

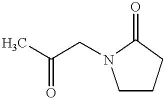

Methylmagnesium chloride (2.7 ml, 3M in tetrahydrofuran, 8.1 mmol) was added to a cooled (−20° C.) solution of the amide from preparation 15 (1.5 g, 8.1 mmol) in tetrahydrofuran (50 ml), and the reaction allowed to warm to room temperature, then stirred for an hour. The mixture was quenched by the addition of aqueous ammonium chloride solution, then extracted with ethyl acetate (3×50 ml). The combined organic solutions were dried (MgSO₄), and evaporated under reduced pressure to give the title compound as an oil, 645 mg, 56%; ¹H NMR (CDCl₃, 400 MHz) δ: 2.07 (m, 2H), 2.17 (s, 3H), 2.42 (t, 2H), 3.42 (t, 2H), 4.10 (s, 2H).

Preparation 17

1-[2-(Hydroxyimino)propyl]-2-pyrrolidinone

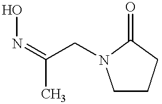

Hydroxylamine hydrochloride (316 mg, 4.55 mmol) and then pyridine (37011, 4.58 mmol) were added to a solution of the amide from preparation 16 (643 mg, 4.55 mmol) in ethanol (30 ml), and the reaction stirred at room temperature for 18 hours. The mixture was evaporated under reduced pressure and the residue purified by column chromatography on silica gel using an elution gradient of dichloromethane:methanol (97:3 to 90:10). The product was triturated with ether to give the title compound as a white solid, 375 mg, 53%; ¹H NMR (DMSOd₆, 400 MHz) δ: 1.60 (s, 3H), 1.87 (m, 2H), 2.20 (t, 2H), 3.19 (t, 2H), 3.78 (s, 2H), 10.77 (s, 1H); LRMS: m/z 157.4 (MH⁺).

Preparation 18

tert-Butyl 1-benzyl-2-oxo-2-[(3-Pyridinylsulfonyl)amino]ethylcarbamate

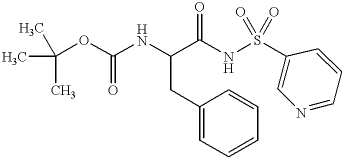

1-(3-Dimethylaminopropyl)-3-ethylcarbodiimide hydrochloride (939 mg, 4.9 mmol), 1-hydroxybenzotriazole hydrate (562 mg, 4.15 mmol), and N-methylmorpholine (952 mg, 9.42 mmol) were added to an ice-cold solution of N-tert-butoxycarbonyl-L-phenylalanine (1.0 g, 3.77 mmol) in dichloromethane (20 ml), and the mixture stirred for 15 minutes. 3-Pyridinesulphonamide (Mon. für Chemie; 72; 77; 1938) (596 mg, 3.77 mmol) was added, and the reaction stirred at room temperature for 24 hours. The mixture was evaporated under reduced pressure and the residue partitioned between ethyl acetate (50 ml) and water (50 ml), and the layers separated. The aqueous layer was extracted with ethyl acetate, then dichloromethane, the combined organic extracts dried (MgSO₄) and evaporated under reduced pressure. The crude product was purified twice by column chromatography on silica gel, using an elution gradient of ethyl acetate:ethanol (100:0 to 90:10) to give the desired product as a white foam, 1.01 g, 66; ¹H NMR (DMSOd₆, 300 MHz) δ: 1.30 (s, 9H), 2.77 (m, 1H), 2.97 (m, 1H), 3.84 (m, 1H), 5.95 (bs, 1H), 6.96 (m, 2H), 7.08 (m, 3H), 7.42 (m, 1H), 8.05 (d, 1H), 8.60 (d, 1H), 8.84 (m, 1H); [α]_{D}=−10° (0.1% solution in methanol).

Preparation 19

(5-Bromo-3-pyridinyl)(phenyl)methanol

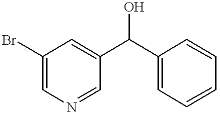

n-Butyl lithium (17 ml, 2.5M in hexanes, 42.5 mmol) was added dropwise to cooled (−78° C.) solution of 3,5-dibromopyridine (10 g, 42.2 mmol) in ether (200 ml), so as to maintain an internal temperature <−70° C. The mixture was then stirred for 15 minutes and a solution of benzaldehyde (4.5 g, 42.5 mmol) in ether (20 ml) was added dropwise, again maintaining the temperature <−70° C. The mixture was stirred for 15 minutes, then allowed to warm to room temperature over an hour. The reaction was quenched by the addition of 0.9M ammonium chloride solution (200 ml), the layers separated, and the aqueous phase extracted with ether. The combined organic extracts were dried (MgSO₄) and evaporated under reduced pressure. The residual yellow oil was purified by column chromatography on silica gel using an elution gradient of dichloromethane:ether (95:5 to 80:20) to give the title compound as a yellow oil, 7.6 g, 68%; ¹H NMR (D₂O, 300 MHz) δ: 5.80 (s, 1H), 7.37 (m, 5H), 7.90 (s, 1H), 8.40 (s, 1H), 8.44 (s, 1H).

Preparation 20

(1S,3R)-3-Aminocyclopentanecarboxamide hydrochloride

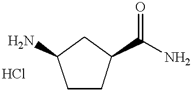

Hydrogen chloride gas was bubbled through an ice-cooled solution of the amide from preparation 10 (438 mg, 1.92 mmol) in dichloromethane (50 ml) for 10 minutes, and the resulting suspension stirred at room temperature for 2 hours. The mixture was purged with nitrogen, then evaporated under reduced pressure. The residue was triturated with ether, to afford the title compound as a solid; ¹H NMR (D₂O, 400 MHz) δ: 1.63-1.82 (m, 3H), 1.92-2.07 (m, 2H), 2.19 (m, 1H), 2.82 (m, 1H), 3.62 (m, 1H).

Preparation 21

3-Amino-N,N-dimethylcyclohexanecarboxamide

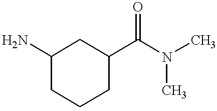

A solution of the amide from preparation 11 (997 mg, 3.69 mmol) in trifluoroacetic acid (8 ml) and dichloromethane (8 ml) was stirred at room temperature for 4 hours. The mixture was concentrated under reduced pressure and the residue partitioned between dichloromethane (25 ml) and sodium bicarbonate solution (25 ml). The pH was adjusted to 9 using sodium hydroxide solution, the layers separated, and the aqueous phase evaporated under reduced pressure. The resulting solid was triturated with hot ethyl acetate, the suspension filtered and the filtrate concentrated under reduced pressure. The crude product was purified by column chromatography on silica gel using dichloromethane:methanol:0.88 ammonia (84:14:2) to afford the title compound as a colourless oil, 346 mg, 55%; ¹H NMR (CDCl₃, 300 MHz) δ: 1.08 (m, 1H), 1.25-1.54 (m, 6H), 1.72 (m, 1H), 1.86 (m, 2H), 2.53-2.75 (m, 2H), 2.96 (s, 3H), 3.03 (s, 3H).

Preparation 22

(5-Methyl-1,3,4-thiadiazol-2-yl)methylamine hydrochloride

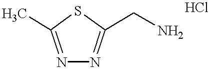

Hydrogen chloride gas was bubbled through an ice-cooled solution of the thiadiazole from preparation 14 (425 mg, 1.85 mmol) in dichloromethane (50 ml) for 15 minutes, and the reaction stirred at room temperature for 1 hour. The mixture was purged with nitrogen, then evaporated under reduced pressure to afford the title compound as a white solid; ¹H NMR (DMSOd₆, 400 MHz) δ: 2.75 (s, 3H), 4.48 (m, 2H), 8.80 (bs, 3H).

Preparation 23

3-Amino-N-methylpropanamide hydrochloride

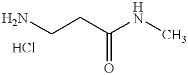

A mixture of the benzyl carbamate from preparation 13 (7.92 g, 33.5 mmol) and 5% palladium on charcoal (800 mg) in ethanol (300 ml) was hydrogenated at 50 psi and room temperature for 4 hours. The reaction mixture was filtered through Arbocel® washing through with ethanol, and 1N hydrochloric acid (36.9 ml, 36.9 mmol) was added to the combined filtrate. This solution was evaporated under reduced pressure and the residue azeotroped with dichloromethane to afford the title compound, 4.66 g, ¹H NMR (DMSOd₆, 300 MHz) δ: 2.46 (t, 2H), 2.60 (s, 3H), 2.95 (m, 2H), 7.98-8.16 (m, 2H).

Preparation 24

1-(2-Aminopropyl)-2-pyrrolidinone

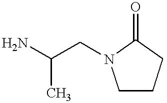

A mixture of the oxime from preparation 17 (375 mg, 2.40 mmol) and platinum oxide (300 mg) in ethanol (20 ml) was hydrogenated at 60 psi and room temperature for 18 hours. Tlc analysis showed starting material remaining, so additional platinum oxide (100 mg) was added and the reaction continued for a further 4 hours. The mixture was filtered through Arbocel®, and the filtrate evaporated under reduced pressure. The crude product was purified by column chromatography on silica gel using an elution gradient of dichloromethane:methanol:0.88 ammonia (95:5:0.5 to 90:10:1) to give the title compound as a clear oil, 170 mg, 50%; ¹H NMR (CDCl₃, 400 MHz) δ: 1.02 (d, 3H), 1.36 (bs, 2H), 2.00 (m, 2H), 2.38 (t, 2H), 3.00-3.16 (m, 2H), 3.21 (m, 1H), 3.35-3.45 (m, 2H); LRMS: m/z 143 (MH⁺).

Preparation 25

N-(2-Amino-3-phenylpropanoyl)-3-pyridinesulphonamide dihydrochloride

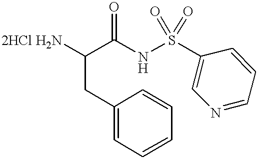

Saturated ethereal hydrochloric acid (40 ml) was added to an ice-cold solution of the sulphonamide from preparation 18 (959 mg, 2.37 mmol) in ethyl acetate (30 ml) and ether (10 ml), and the solution stirred at room temperature for 18 hours. The reaction mixture was concentrated under reduced pressure and the residue azeotroped with dichloromethane (3×) to afford the title compound as a white solid, 959 mg; ¹H NMR (DMSOd₆, 300 MHz) δ: 3.23-3.50 (m, 1H), 3.70-3.98 (m, 1H), 4.13 (m, 1H), 7.05 (m, 2H), 7.20 (m, 3H), 7.78 (m, 1H), 8.36 (d, 1H), 8.44 (bs, 2H), 8.95 (d, 1H), 9.02 (s, 1H); [α]_{D}=+138° (0.5% solution in methanol).

Preparation 26

(5-Amino-3-pyridinyl)(phenyl)methanol

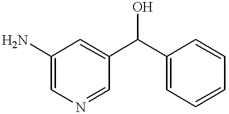

A mixture of the bromide from preparation 19 (2.0 g, 7.60 mmol) and copper (II) sulphate pentahydrate (350 mg, 1.40 mmol) in 0.88 ammonia (1 8 ml) was heated at 135° C. in a sealed vessel for 24 hours. Sodium hydroxide solution (1N, 10 ml) was added to the cooled solution, and the mixture was then extracted with ether (6×). The combined organic extracts were dried (MgSO₄), and concentrated under reduced pressure. The resulting precipitate was filtered, washed with ether and dried to give the title compound as a solid, 1.25 g, 83%; m. p. 92-94° C.; ¹H NMR (DMSOd₆, 300 MHz) δ: 5.22 (s, 2H), 5.59 (d, 1H), 5.86 (d, 1H), 6.83 (s, 1H), 7.20 (m, 1H), 7.34 (m, 4H), 7.78 (m, 2H).

Preparation 27

5-Benzyl-3-pyridinylamine

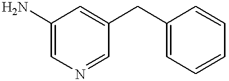

A mixture of the alcohol from preparation 26 (700 mg, 3.5 mmol) and 5% palladium on charcoal (70 mg) in hydrochloric acid (5 ml, 1N) and ethanol (20 ml) was hydrogenated at 30 psi and room temperature for 6 hours. The mixture was filtered through Arbocel®), and the filtrate concentrated under reduced pressure. The residue was basified using aqueous sodium bicarbonate solution, extracted with dichloromethane (3×), and the combined organic extracts dried (MgSO₄), and evaporated under reduced pressure. The crude product was purified by column chromatography on silica gel using dichloromethane:methanol:0.88 ammonia (92:8:0.4) as eluant, to give the title compound as a solid, 500 mg, 78%; mp 107-109° C.; ¹ H NMR (CDCl₃, 300 MHz) δ: 3.61 (bs, 2H), 3.94 (s, 2H), 6.78 (s, 1H), 7.24 (m, 5H), 7.98 (s, 2H).

Preparation 28

5-Amino-1-benzyl-2(1H)-pyridinone

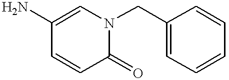

A mixture of 1-benzyl-5-nitro-1H-pyridin-2-one (Justus Liebigs Ann. Chem. 484; 1930; 52) (1.0 g, 4.35 mmol), and granulated tin (3.5 g, 29.5 mmol) in concentrated hydrochloric acid (14 ml) was heated at 90° C. for 1.5 hours. The cooled solution was diluted with water, neutralised using sodium carbonate solution, and extracted with ethyl acetate (250 ml in total). The combined organic extracts were filtered, dried (MgSO₄), and evaporated under reduced pressure to give the title compound as a pale green solid, (turned blue with time), 440 mg, 51%; ¹H NMR (CDCl₃, 250 MHz) δ: 4.12-4.47 (bs, 2H), 5.00 (s, 2H), 6.31 (d, 1H), 6.86 (s, 1H), 7.07 (m, 1H), 7.14-7.42 (m, 5H).

Preparation 29

Cis-(4-Aminocyclohexyl)methanol

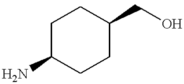

Lithium aluminium hydride (14 ml, 1M solution in tetrahydrofuran, 14 mmol) was added dropwise to an ice-cooled solution of cis-4-aminocyclohexanecarboxylic acid (1.33 g, 9.29 mmol) in tetrahydrofuran (50 ml), and once addition was complete, the reaction was heated under reflux for 6 hours. The resulting suspension was cooled to 5° C., and water (0.6 ml), aqueous sodium hydroxide solution (1.1 ml, 2M), then water (0.6 ml) were added sequentially. The resulting suspension was filtered, and the filtrate evaporated under reduced pressure to give an oil, which was used without further purification; ¹H NMR (CDCl₃, 300 MHz) δ: 1.40-1.80 (m, 12H), 3.00 (m, 1H), 3.55 (d, 2H); LRMS: m/z 130.2 (MH⁺).

Preparation 30

2-Amino-4-butylpyridine

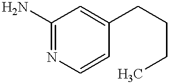

A mixture of 4-butylpyridine (5.0 g, 37.0 mmol) and 95% sodium amide (1.7 g, 40.7 mmol) in xylene (10 ml) was heated at 150° C. for 18 hours. The cooled mixture was diluted with ether (100 ml) and extracted with 2N hydrochloric acid (twice). The aqueous extracts were basified using sodium hydroxide solution, and re-extracted with ether. These combined organic extracts were dried (MgSO₄) and evaporated under reduced pressure. The residual oil was purified by column chromatography on silica gel using dichloromethane:methanol:0.88 ammonia (97:3:0.15) as eluant, to afford the title compound as a crystalline solid, 2.1 g, 38%; ¹H NMR (CDCl₃, 300 MHz) δ: 0.96 (t, 3H), 1.38 (m, 2H), 1.60 (m, 2H), 2.52 (t, 2H), 4.38 (bs, 2H), 6.38 (s, 1H), 6.55 (d, 1H), 7.98 (d, 1H);

Anal. Found: C, 72.01; H, 9.47; N, 18.53. C₉H₁₄N₂requires C, 71.96; H, 9.39; N, 18.65%.

Preparation 31

5-(Cyclopropylmethyl)-1,3,4-thiadiazol-2-amine

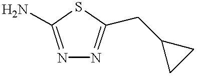

Oxalyl chloride (3.13 ml, 35.9 mmol) and N,N-dimethylformamide (1 drop) were added to a solution of cyclopropylacetic acid (3 g, 29.9 mmol) in dichloromethane (30 ml), and the reaction stirred at room temperature for 18 hours. The mixture was concentrated under reduced pressure and azeotroped with dichloromethane to give a brown oil. A mixture of this intermediate acid chloride (887 mg, 7.48 mmol) and thiosemicarbazide (455 mg, 4.99 mmol) were heated at 70° C. for 18 hours, then cooled. Water was added, the mixture basified to pH 9 using 50% aqueous sodium hydroxide solution, and the resulting precipitate filtered and dried, to give the title product, 410 mg, 53%; ¹H NMR (CD₃OD, 400 MHz) δ: 0.28 (m, 2H), 0.60 (m, 2H), 1.02 (m, 1H), 2.77 (d, 2H); LRMS: m/z 155.2 (MH⁺).

Preparation 32

(1R,2R,4S)-4-[({1-[2-(tert-Butoxycarbonyl)pentyl]cyclopentyl}carbonyl)amino]-2-butylcyclohexanecarboxamide

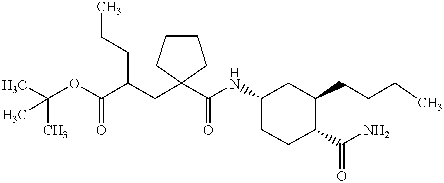

The product from Preparation 49 (65 mg, 0.14 mmol) was taken up in DCM (2 mls) and 1,1′-carbonyidiimidazole (23 mg, 0.15 mmol) added in one portion. 0.88 aqueous ammonia solution (0.5 mls) was added and the mixture stirred for 16 h. The volatiles were removed under reduced pressure, and the resulting suspension treated with saturated aqueous NaHCO₃ solution (5 mls). The organics were extracted with EtOAc (2×), dried (MgSO₄) and evaporated to give an oil, which was purified by column chromatography using 19:1 (DCM:MeOH) as eluant to provide the title product (43 mg); ¹H NMR (CDCl₃, 400 MHz) δ: 0.81 (t, 6H), 1.02-1.28 (m, 37H), 2.22 (m, 1H), 4.13 (m, 1H), 5.42-5.97 (m, 3H).

Preparation 33

tert-Butyl 2-{[1-({[1-(hydroxymethyl)cyclopentyl]amino}carbonyl)-cyclopentyl]methyl}pentanoate

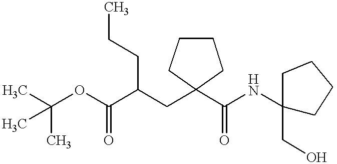

1-(3-Dimethylaminopropyl)-3-ethylcarbodiimide hydrochloride (41 mg, 0.21 mmol), 1-hydroxybenzotriazole hydrate (27 mg, 0.2 mmol), N-methylmorpholine (35 μl, 0.31 mmol) and finally 1-amino-1-cyclopentanemethanol (ex Aldrich Chemical Co.) (25 mg, 0.22 mmol) were added to a solution of the acid from preparation 1 (150 mg, 0.53 mmol) in N,N-dimethylformamide (3 m[), and the reaction stirred at 90° C. for 18 hours. The cooled solution was diluted with ethyl acetate (90 ml), washed with water (3×25 ml), and brine (25 ml), then dried (MgSO₄) and evaporated under reduced pressure. The crude product was purified by chromatography on silica gel, using ethyl acetate:pentane (30:70) as the eluant to afford the title compound, 38 mg, 57%; ¹H NMR (CDCl₃, 400 MHz) δ: 0.88 (t, 3H), 1.29 (m, 3H), 1.41-1.78 (m, 26H), 1.78-1.98 (m, 4H), 2.04 (m, 1H), 2.26 (m, 1H), 3.59 (dd, 1H), 3.70 (dd, 1H), 4.80 (t, 1H), 5.81 (s, 1H); LRMS: m/z 380 (MH⁻).

Preparations 34 to 43

Compounds of formula IVc, i.e. compounds of general formula IV where Prot is tert-butyl and R¹ is propyl, were prepared from the title product from Preparation 1 and the amine indicated, following a similar procedure to that described in Preparation 33.

| | |
|---|---|
| | (IVc) |
| 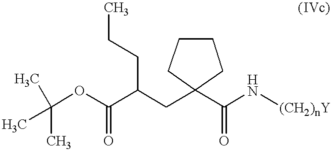 | |

| Prep | —(CH₂)ₙY | Prec. amine | Data |
|---|---|---|---|
| 34 | 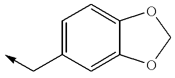 | piperonylamine (ex Aldrich Chemical Co.) | ¹H NMR (CDCl₃, 400 MHz) δ: 0.85 (t, 3H), 1.26 (m, 4H), 1.42 (s, 9H), 1.46 (m, 2H), 1.59-1.75 (m, 5H), 1.95 (m, 2H), 2.06 (m, 1H), 2.22 (m, 1H), 4.26 (dd, 1H), 4.39 (dd, 1H), 5.95 (m, 3H), 6.78 (m, 3H). LRMS: m/z 418.3 (MH⁺) |
| 35¹ | 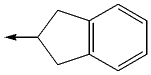 | 2-aminoindan hydrochloride (ex Aldrich Chemical Co.) | ¹H NMR (CDCl₃, 400 MHz) δ: 0.87 (t, 3H), 1.25 (m, 3H), 4H), 1.42 (m, 12H), 1.56-1.70 (m, 4H), 1.90 (m, 2H), 2.02 (m, 1H), 2.22 (m, 1H), 2.80 (m, 2H), 3.35 (m, 2H), 4.76 (m, 1H), 5.86 (d, 1H), 7.19 (m, 4H). LRMS: m/z 400.3 (MH⁺) |
| 36² | 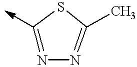 | 2-amino-5- methyl-1,3,4- thiadiazole (ex Lancaster) | ¹H NMR (CDCl₃, 400 MHz) δ: 0.82 (t, 3H), 1.20-1.85 (m, 20H), 2.18 (m, 4H), 2.67 (s, 3H), 9.80 (bs, 1H). LRMS: m/z 382.3 (MH⁺) |
| 37² | 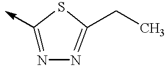 | 2-amino-5-ethyl- 1,3,4-thiadiazole (ex Lancaster) | ¹H NMR (CDCl₃, 300 MHz) δ: 0.82 (t, 3H), 1.20-1.80 (m, 22H), 1.84 (m, 1H), 2.20 (m, 4H), 3.04 (q, 2H), 9.10 (bs, 1H). LRMS: m/z 396.2 (MH⁺) |
| 38 | 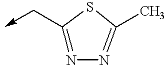 | Prep 22 | ¹H NMR (CDCl₃, 300 MHz) δ: 0.84 (t, 3H), 1.20-1.38 (m, 4H), 1.42 (s, 9H), 1.44-1.76 (m, 7H), 1.95-2.12 (m, 3H), 2.20 (m, 1H), 2.76 (s, 3H), 4.74 (dd, 1H), 4.82 (dd, 1H), 6.54 (bs, 1H). LRMS: m/z 396.2 (MH⁺) |
| 39^{1,2} | 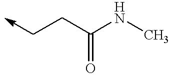 | Prep 23 | ¹H NMR (CDCl₃, 300 MHz) δ: 0.88 (t, 3H), 1.21-1.38 (m, 3H), 1.40-1.70 (m, 17H), 1.88-2.04 (m, 3H), 2.20 (m, 1H), 2.39 (t, 2H), 2.80 (d, 3H), 3.53 (m, 2H), 6.13 (bs, 1H), 6.40 (m, 1H). LRMS: m/z 369.5 (MH⁺) |
| 40² | 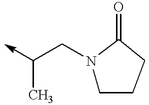 | Prep 24 | ¹H NMR (CDCl₃, 300 MHz) δ: 0.82 (m, 3H), 1.16 (2xd, 3H), 1.20-1.72 (m, 21H), 1.83 (m, 1H), 1.98 (m, 3H), 2.17 (m, 1H), 2.38 (m, 2H), 1.96 (m, 1H), 3.34 (m, 1H), 3.54-3.62 (m, 2H), 4.15-4.20 (m, 1H), 6.21-6.35 (2xbd, 1H). LRMS: m/z 409.3 (MH⁺). |
| 41² | 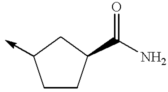 | Prep 20 | ¹H NMR (CDCl₃, 400 MHz) δ: 0.82 (t, 3H), 1.19-1.38 (m, 4H), 1.42 (m, 12H), 1.60 (m, 3H), 1.74-2.02 (m, 10H), 2.18 (m, 1H), 2.78 (m, 1H), 4.38 (m, 1H), 5.32 (bs, 1H), 5.57 (bs, 1H), 7.28 (bs, 1H). LRMS: m/z 395 (MH⁺) |
| 42² | 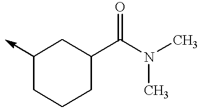 | Prep 21 | ¹H NMR (CDCl₃, 300 MHz) δ: 0.86 (t, 3H), 1.18-1.78 (m, 25H), 1.84-2.03 (m, 6H), 2.22 (m, 1H), 2.68 (m, 1H), 2.96 (s, 3H), 3.03 (s, 3H), 3.84 (m, 1H), 5.78 (m, 1H). LRMS: m/z 437.7 (MH⁺) |
| 43² | 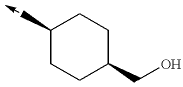 | Prep 29 | ¹H NMR (CDCl₃, 300 MHz) δ: 0.85 (t, 3H), 1.20-1.79 (m, 30H), 1.90 (m, 2H), 2.05 (m, 1H), 2.24 (m, 1H), 3.56 (m, 2H), 4.04 (m, 1H), 5.82 (bd, 1H). LRMS: m/z 396.4 (MH⁺) |

¹= reaction conducted at room temperature
²= Methanol:dichloromethane was used as the column eluant

Preparation 44

tert-Butyl 2-{[1-({[2-(1H-indol-3-yl)ethyl]amino}carbonyl)cyclopentyl]-methyl}pentanoate

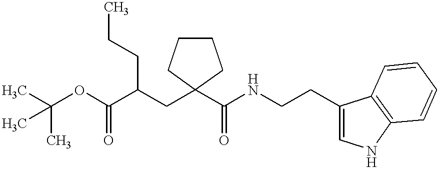

The title compound was obtained as a pale yellow oil in 80% yield from the acid from preparation 1 and tryptamine, following a similar procedure to that described in preparation 33, except the reaction was performed in dichloromethane at room temperature; ¹H NMR (CDCl₃, 400 MHz) δ: 0.86 (t, 3H), 1.26 (m, 3H), 1.42 (m, 11H), 1.50-1.69 (m, 6H), 1.83 (m, 1H), 1.90-2.05 (m, 2H), 2.22 (m, 1H), 2.99 (t, 3H), 3.60 (m, 2H), 5.78 (m, 1H), 7.06 (s, 1H), 7.14 (m, 1H), 7.20 (m, 1H), 7.38 (d, 1H), 7.63 (d, 1H), 8.02 (bs, 1H); LRMS: m/z427.5 (MH⁺).

Preparation 45

tert-Butyl 2-[(1-{[(3S)-1-benzylpyrrolidinyl]amino}cyclopentyl)methyl]pentanoate

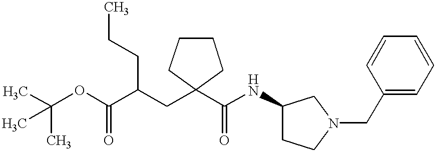

The title compound was obtained quantitatively from the acid from preparation 1 and (3S)-1-benzyl-3-aminopyrrolidine (ex. Aldrich Chemical Co.), following a similar procedure to that described in preparation 44; ¹H NMR (CDCl₃, 300 MHz) δ: 0.84 (t, 3H), 1.10-1.76 (m, 21H), 1.90-2.05 (m, 3H), 2.20-2.38 (m, 3H), 2.58 (m, 2H), 2.84 (m, 1H), 3.62 (s, 2H), 4.45 (m, 1H), 6.02 (m, 1H), 7.33 (m, 5H).

Preparation 46

tert-Butyl 2-{[1-({[cis-2-phenylcyclopropyl]amino}carbonyl)-cyclopentyl]methyl}pentanoate

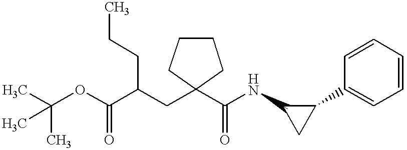

1-(3-Dimethylaminopropyl)-3-ethylcarbodiimide hydrochloride (81 mg, 0.42 mmol), N-methylmorpholine (0.15 ml, 1.06 mmol) and 1-S-amino-2-R-phenyl cyclopropane hydrochloride (J. Med. Chem., 1986, 29, 2044) (60 mg, 0.35 mmol) were added to a solution of the acid from preparation 1 (100 mg, 0.35 mmol) in dichloromethane (10 ml), and the reaction stirred at room temperature for 18 hours. The reaction mixture was evaporated under reduced pressure and the residue purified by column chromatography on silica gel using an elution gradient of dichloromethane:methanol (98:2 to 95:5) to afford the title compound as a yellow oil, 85 mg, 55%; ¹H NMR (CDCl₃, 300 MHz) δ: 0.88 (t, 3H), 1.16 (m, 1H), 1.20-1.58 (m, 16H), 1.63 (m, 5H), 1.90-2.14 (m, 4H), 2.23 (m, 1H), 2.90 (m, 1H), 6.00 (m, 1H), 7.19 (m, 3H), 7.24 (m, 2H); LRMS: m/z400 (MH⁺).

Preparation 47

tert-Butyl 2-{[1-({[2-(2-oxo-1-piperidinyl)ethyl]amino}carbonyl)cyclopentyl]-methyl}pentanoate

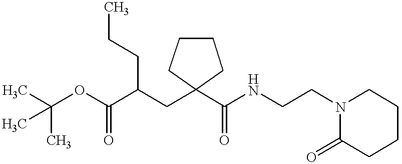

Hydrazine monohydrate (34 μl, 0.70 mmol) was added to a solution of the compound from preparation 6 (171 mg, 0.63 mmol) in ethanol (10 ml), and the reaction heated under reflux for 5 hours. The cooled mixture was filtered, the filtrate concentrated under reduced pressure, the residue suspended in dichloromethane, and the suspension re-filtered. The resulting filtrate was concentrated under reduced pressure, and the residue purified by column chromatography on silica gel using dichloromethane:methanol:0.88 ammonia (90:10:1) as eluant to give the amine, 16 mg. The acid from preparation 1 (32 mg, 0.11 mmol), 1-(3-dimethylaminopropyl)-3-ethylcarbodiimide hydrochloride (25 mg, 0.13 mmol), 1-hydroxybenzotriazole hydrate (17 mg, 0.13 mmol), and N-methylmorpholine (25 μl, 0.23 mmol) were added to a solution of this amine in N,N-dimethylformamide (2 ml), and the reaction stirred at room temperature for 18 hours. The mixture was partitioned between ethyl acetate and water, and the layers separated. The organc phase was washed with water (2×), dried (MgSO₄), and evaporated under reduced pressure. The residual oil was purified by column chromatography on silica gel using an elution gradient of dichloromethane:methanol (98.5:1.5 to 95:5) to afford the title compound as an oil, 43 mg, 17%; ¹H NMR (CDCl₃, 400 MHz) δ: 0.82 (t, 3H), 1.22 (m, 3H), 1.38-1.65 (m, 17H), 1.58 (m, 4H), 1.95 (m, 3H), 2.17 (m, 1H), 2.37 (m, 2H), 3.30 (m, 2H), 3.38 (m, 2H), 3.50 (m, 2H), 6.76 (m, 1H); LRMS: m/z 409.2 (MH⁺)

Preparation 48

Ethyl (1R,2R,4S)-4-[({1-[2-(tert-butoxycarbonyl)pentyl]cyclopentyl}carbonyl)-amino]-2-butylcyclohexanecarboxylate 72846

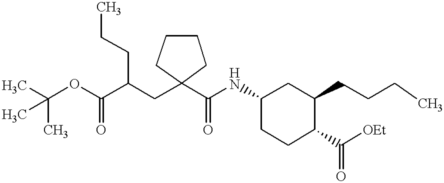

A mixture of the acid from preparation 1 (109 mg, 0.38 mmol), (1R,2R,4S)-4-amino-2-butyl-cyclohexanecarboxylic acid ethyl ester hydrochoride (WO, 9009374), (101 mg, 0.38 mmol), 1-(3-dimethylaminopropyl)-3-ethylcarbodiimide hydrochloride (95 mg, 0.50 mmol), 1-hydroxybenzotriazole hydrate (60 mg, 0.40 mmol) and triethylamine (0.12 ml, 0.87 mmol) in dichloromethane (3 ml), was stirred at room temperature for 16 hours. The mixture was evaporated under reduced pressure, the residue treated with sodium bicarbonate solution and extracted with ethyl acetate. The combined organic extracts were dried (MgSO₄) and evaporated under reduced pressure to give a gum. The crude product was purified by column chromatography on silica gel using ethyl acetate:pentane (50:50) as eluant, and azeotroped with dichloromethane to afford the title compound, 190 mg; ¹H NMR (CDCl₃, 300 MHz) δ: 0.88 (m, 6H), 1.20-1.40 (m, 13H), 1.40-2.10 (m, 25H), 2.16-2.30 (m, 2H), 4.18 (m, 3H), 5.83 (d, 1H).

Preparation 49

(1R, 2R,4S)-4-[({1-[2-(tert-Butoxycarbonyl)pentyl]cyclopentyl}carbonyl)amino]-2-butylcyclohexanecarboxylic acid

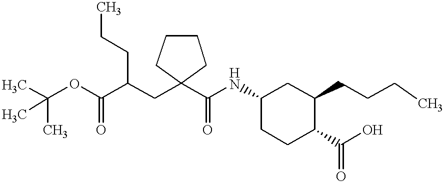

A mixture of the ethyl ester from preparation 48 (190 mg, 0.39 mmol) and 1N sodium hydroxide solution (0.85 ml, 0.85 mmol) in methanol (1.5 ml) was stirred at room temperature for 22 hours. The reaction mixture was acidifed to pH 1 using hydrochloric acid (2N), then partitioned between ethyl acetate and water. The layers were separated, and the organic phase was dried (MgSO₄) and evaporated under reduced pressure to afford the title compound, 130 mg, 72%; ¹H NMR (CDCl₃, 300 MHz) δ: 0.86 (m, 6H), 1.20-2.12 (m, 36H), 2.24 (m, 2H), 4.18 (m, 1H), 5.82 (d, 1H); LRMS: m/z464 (M−H)⁻.

Preparation 50

tert-Butyl (2R)-2-{[1-({[5-(cyclopropylmethyl)-1,3,4-thiadiazol-2-yl]amino}carbonyl)cyclopentyl]methyl}pentanoate

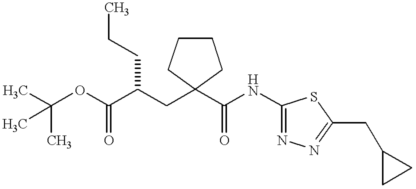

The title compound was prepared from the acid from preparation 2 and the amine from preparation 31, in 65% yield, following the procedure described in preparation 33; ¹H NMR (CDCl₃, 400 MHz) δ: 0.35 (m, 2H), 0.63 (m, 2H), 0.80 (m, 3H), 1.10 (m, 1H), 1.20-1.94 (m, 20H), 2.19 (m, 4H), 2.93 (t, 2H), 3.50 (s, 1H); LRMS: m/z 422.4 (MH⁺); [α]_{D}=−14.15° (c=0.082, methanol).

Preparation 51

tert-Butyl (2R)-2-{[1-({[5-(ethoxymethyl)-1,3,4-thiadiazol-2-yl]amino}carbonyl)-cyclopentyl]methyl}pentanoate

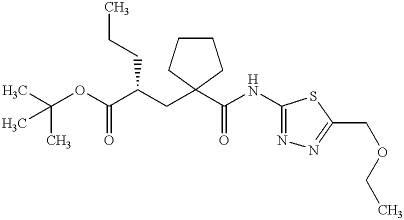

The title compound was prepared from the acid from Preparation 2 and the title product from Preparation 97 in 51% yield, following the procedure described in preparation 33; ¹H NMR (CDCl₃, 400 MHz) δ: 1.10-1.78 (m, 25H), 1.82 (m, 1H), 2.19 (m, 5H), 3.48 (s, 1H), 4.82 (s, 2H), 10.16 (brs, 1H); LRMS: m/z 426.4 (MH⁺); [α]_{D}=−12.50° (c=0.08, methanol).

Preparation 51a

tert-Butyl-(2R)-2-[(1-{[(5-ethyl- 1,3,4-thiadiazol-2-yl)amino]carbonyl}cyclopentyl) methyl]pentanoate

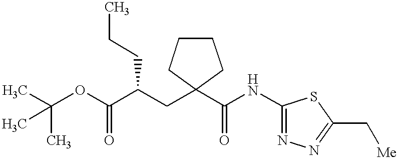

To a mixture of dichloromethane (2.39 L) and pyridine (2.39 L) at −14° C. was added thionyl chloride (135 ml, 1.85 mol, 1.1 eq) with stirring over a period of 1 hour under a nitrogen atmosphere. After stirring the resultant orange solution for 5 minutes, a solution of the product from Preparation 2 (477 g, 1.67 mol) in dichloromethane (477 ml) was added over a period of 45 minutes, causing the reaction mixture to become turbid. After stirring the reaction mixture for 3 hours, triethylamine (424 g, 4.19 mol) was added over a period of 20 minutes, followed by 4-dimethylaminopyridine (20.5 g, 168 mmol). To the resultant mixture was then added 2-amino-5-ethyl-1,3,4-thiadiazole (ex. Lancaster) (282 g, 2.18 mol) in 3 portions over a period of 10 minutes. The reaction was then allowed to warm to ambient temperature and was stirred for 43 hours. n-Heptane (2.4 L) and deionised water (1 L) were then added to the reaction mixture. Concentrated hydrochloric acid (3.56 L) was then added with stirring over a period of 0.5 hours whilst cooling in an ice-water bath. The layers were then separated and to the organic layer was added deionised water (2 L) and concentrated hydrochloric acid (250 ml) with stirring. The layers were separated again, and to the organic layer was added deionised water (2 L) and concentrated hydrochloric acid (250 ml) with stirring. To the organic layer was then added saturated aqueous potassium carbonate solution (1 L) with stirring. The organic phase was collected and was then washed with saturated brine (2×1 L). The resultant solution was then concentrated under vacuum to give the title compound (546 g, 1.38 mol, 85% yield) as a viscous, dark brown oil that was used in the next step without further purification; LRMS (negative APCI) : m/z (M−H]⁻ 394; ¹H-NMR (CDCl₃, 300 MHz) δ: 0.76 (t, 3H), 1.08-1.23 (m, 3H), 1.23-1.27 (m, 2H), 1.32 (s, 9H), 1.34-1.42 (m, 2H), 1.42-1.53 (m, 4H), 1.53-1.68 (m, 2H), 1.87 (dd, 1H), 1.95-2.06 (m, 1H), 2.06-2.24 (m, 3H), 2.98 (q, 2H), 12.15 (bs, 1H).

Preparation 52

Benzyl 2-({1-(3-pyridinylamino)carbonyl]cyclopentyl}methyl)pentanoate

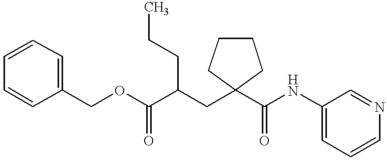

Triethylamine (0.11 ml, 0.78 mmol) was added to a mixture of the acid chloride from preparation 3 (200 mg, 0.60 mmol) and 2-aminopyridine (61 mg, 0.65 mmol) in dichloromethane (3 ml), and the reaction stirred at room temperature for 16 hours. The mixture was evaporated under reduced pressure, the residue partitioned between sodium bicarbonate solution (5 ml) and ethyl acetate (20 ml), and the layers separated. The organic phase was dried (MgSO₄), and evaporated under reduced pressure to give a gum. The crude product was purified by column chromatography on silica gel using ethyl acetate as eluant, to afford the title compound, 130 mg; ¹H NMR (CDCl₃, 400 MHz) δ: 0.82 (t, 3H), 1.21 (m, 3H), 1.40 (m, 1H), 1.43-1.72 (m, 6H), 1.81 (d, 1H), 1.98 (m, 1H), 2.18 (m, 1H), 2.24 (m, 1H), 2.46 (m, 1H), 4.98 (m, 2H), 7.20-7.38 (m, 6H), 7.42 (s, 1H), 8.06 (d, 1H), 8.35 (d, 1H), 8.56 (s, 1H).

Preparations 53 to 56

Compounds of formula IVd, i.e. compounds of general formula IV where Prot is benzyl and R¹ is propyl, were prepared from the acid chloride from Preparation 3 and the amine indicated, following a similar procedure to that described in preparation 52.

| | |
|---|---|
| | (IVd) |
| 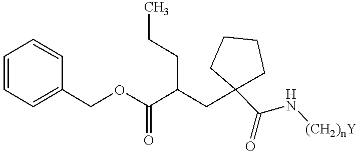 | |

| Prep | —(CH₂)ₙY | Prec. amine | Data |
|---|---|---|---|
| 53¹ | 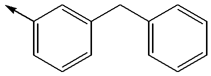 | ex Trans World Chemicals | ¹H NMR (CDCl₃, 300 MHz) δ: 0.84 (t, 3H), 1.24 (m, 2H), 1.40-1.76 (m, 7H), 1.84 (dd, 1H), 1.98 (m, 1H), 2.19 (dd, 1H), 2.28 (m, 1H), 2.56 (m, 1H), 3.98 (s, 2H), 4.99 (dd, 2H), 6.98 (d, 1H), 7.19-7.42 (m, 15H). |
| 54 | 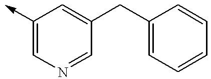 | Prep 27 | ¹H NMR (CDCl₃, 300 MHz) δ: 0.85 (t, 3H), 1.24 (m, 3H), 1.39-1.78 (m, 6H), 1.82 (dd, 1H), 1.98 (m, 2H), 2.20 (dd, 1H), 2.25 (m, 1H), 2.50 (m, 1H), 3.98 (s, 2H), 4.98 (dd, 2H), 7.18-7.40 (m, 10H), 7.45 (s, 1H), 7.98 (s, 1H), 8.23 (s, 1H), 8.42 (s, 1H). |
| 55 | 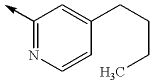 | Prep 30 | ¹H NMR (CDCl₃, 400 MHz) δ: 0.80 (t, 3H), 0.92 (t, 3H), 1.21 (m, 2H), 1.30-1.70 (m, 12H), 1.82 (dd, 1H), 2.04 (m, 1H), 2.20 (m, 2H), 2.50 (m, 1H), 2.58 (t, 2H), 4.98 (dd, 2H), 6.83 (d, 1H), 7.30 (m, 5H), 7.90 (s, 1H), 8.08 (s, 1H), 8.15 (d, 1H). |
| 56² | 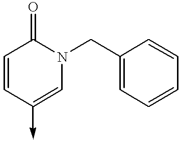 | Prep 28 | ¹H NMR (CDCl₃, 300 MHz) δ: 0.84 (t, 3H), 1.25 (m, 2H), 1.27-1.99 (m, 10H), 2.07-2.30 (m, 2H), 2.47 (m, 1H), 4.99 (s, 2H), 5.10 (dd, 2H), 6.59 (d, 1H), 7.15 (d, 1H), 7.34 (m, 11H), 8.10 (s, 1H). |

¹= dichloromethane used as the column eluant
²= N-methylmorpholine was used as the base

Preparation 57

Benzyl 2-({1-[({1-benzyl-2-oxo-2-[(3-pyridinylsulfonyl)amino]ethyl}amino)-carbonyl]cyclopentyl}methyl)pentanoate

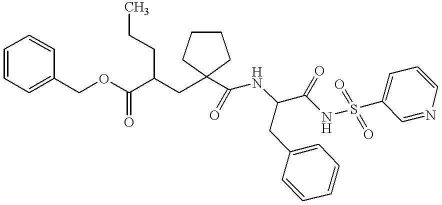

The amine hydrochloride from preparation 25 (828 mg, 2.19 mmol) and N-methylmorpholine (2.21 g, 21.9 mmol) were added to an ice-cold solution of the acid chloride from preparation 3 (737 mg, 2.19 mmol) in dichloromethane (50 ml), and the reaction stirred at room temperature for 24 hours. The reaction mixture was evaporated under reduced pressure, the residue partitioned between ethyl acetate (50 ml) and water (50 ml), and the layers separated. The organic phase was washed with brine (25 ml), dried (MgSO₄) and evaporated under reduced pressure. The crude product was purified by column chromatography on silica gel using an elution gradient of ethyl acetate:methanol (100:0 to 95:5) to give the title compound as a cream foam, 975 mg, 73%; ¹H NMR (CDCl₃, 300 MHz) δ: 0.72 (m, 3H), 0.94-2.20 (m, 17H), 2.84 (m, 1H), 3.00 (m, 1H), 4.18 (m, 1H), 5.00 (m, 2H), 6.95 (m, 2H), 7.02 (m, 3H), 7.38 (m, 6H), 8.06 (m, 1H), 8.60 (m, 1H), 8.87 (s, 1H).

Preparation 58

cis-Benzyl 2-({1-[({4-[(dimethylamino)carbonyl]cyclohexyl}amino)carbonyl]-cyclopentyl}methyl)pentanoate

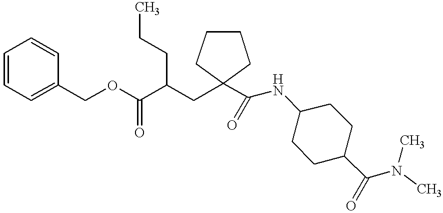

A mixture of cis-4-{[(1-{2-[(benzyloxy)carbonyl]pentyl}cyclopentyl)carbonyl]-amino}cyclohexanecarboxylic acid (EP 274234, Example 310) (200 mg, 0.45 mmol), 1-(3-dimethylaminopropyl)-3-ethylcarbodiimide hydrochloride (112 mg, 0.58 mmol), 1-hydroxybenzotriazole hydrate (70 mg, 0.46 mmol) and dimethylamine (0.56 ml, 2M in tetrahydrofuran, 1.12 mmol) in dichloromethane (5 ml) was stirred at room temperature for 16 hours. The mixture was concentrated under reduced pressure and the residue partitioned between sodium bicarbonate solution and ethyl acetate, and the layers separated. The organic phase was dried (MgSO₄) and evaporated under reduced pressure to give a gum. The crude product was purified by column chromatography on silica gel using ethyl acetate as eluant to afford the title compound, 150 mg; ¹H NMR (CDCl₃, 300 MHz) δ: 0.82 (t, 3H), 1.22 (m, 3H), 1.32-1.88 (m, H), 2.00 (m, 4H), 2.40 (m, 1H), 2.60 (m, 1H), 2.97 (s, 3H), 3.04 (s, 3H), 4.04 (m, 1H), 5.12 (s, 2H), 5.80 (bd, 1H), 7.37 (m, 5H).

Preparation 59

cis-Benzyl 2-({1-[({4-[(methylamino)carbonyl]cyclohexyl}amino)carbonyl]-cyclopentyl}methyl)pentanoate

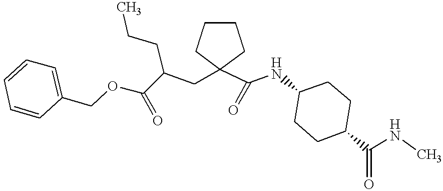

The title compound was prepared in 49% yield from cis-4-{[(1-{2-[(benzyloxy)carbonyl]pentyl}cyclopentyl)carbonyl]amino}cyclohexanecarboxylic acid (EP 274234, Example 310) and methylamine (2M in tetrahydrofuran), following the procedure described in preparation 58; ¹H NMR (CDCl₃, 300 MHz) δ: 0.82 (t, 3H), 1.17-2.12 (m, 22H), 2.21 (m, 1H), 2.41 (m, 1H), 2.80 (d, 3H), 4.00 (m, 1H), 5.12 (s, 2H), 5.61 (m, 1H), 5.79 (d, 1H), 7.38 (m, 5H).

Preparation 60

tert-Butyl 2-[(1-{[(2-{[(benzyloxy)carbonyl]amino}ethyl)amino]carbonyl}-cyclopentyl)methyl]pentanoate

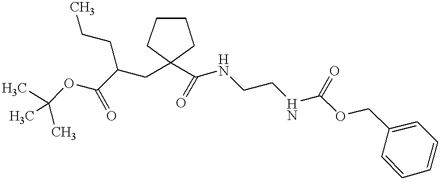

The title compound was obtained as a yellow oil in 55% yield, from the acid from preparation 1 and N-benzyloxycarbonyl-1,2-diaminoethane (ex. Aldrich Chemical Co.) following a similar procedure to that described in preparation 33; ¹H NMR (CDCl₃, 400 MHz) δ: 0.84 (t, 3H), 1.20-1.38 (m, 3H), 1.40-1.74 (m, 17H), 1.90 (m, 2H), 2.04 (m, 1H), 2.20 (m, 1H), 3.32 (m, 3H), 3.44 (m, 1H), 5.10 (s, 2H), 5.61 (m, 1H), 6.20 (m, 1H), 7.36 (m, 5H).

Preparation 61

tert-Butyl 2-[(1-{[(2-aminoethyl)amino]carbonyl}cyclopentyl)methyl]pentanoate

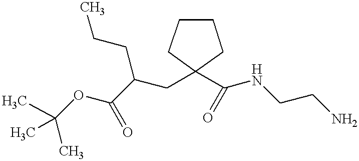

A mixture of the carbamate from preparation 60 (1.43 g, 3.10 mmol) and 10% palladium on charcoal (200 mg) in ethanol (8 ml) was hydrogenated at room temperature and 1 atm for 18 hours. The reaction mixture was filtered through Arbocel®, and the filtrate evaporated under reduced pressure to afford the title compound, 920 mg, 92%; ¹H NMR (CDCl₃, 400 MHz) δ: 0.84 (t, 3H), 1.20-1.38 (m, 3H), 1.40-1.54 (m, 12H), 1.61 (m, 5H), 1.92-2.12 (m, 3H), 2.20 (m, 1H), 2.98 (m, 2H), 3.38 (m, 1H), 3.42 (m, 1H), 3.97 (m, 2H), 6.65 (m, 1H); LRMS: m/z 326.8 (M⁺).

Preparation 62

Benzyl 2-[(1-{[(1-benzyl-6-oxo-1,6-dihydro-3-pyridinyl)amino]carbonyl}cyclopentyl)-methyl]-4-methoxybutanoate

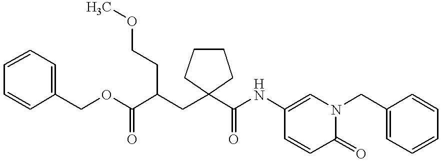

Oxalyl chloride (0.26 ml, 3.0 mmol) was added to an ice-cooled solution of 1-{2-[(benzyloxy)carbonyl]-4-methoxybutyl}cyclopentanecarboxylic acid (EP 274234, Example 15) (1.0 g, 3.0 mmol) and N,N-dimethylformamide (2 drops) in dichloromethane (20 ml), and the reaction stirred at room temperature for 2 hours. The solution was concentrated under reduced pressure and the residue azeotroped with dichloromethane (3×10 ml). The product was dissolved in dichloromethane (20 ml), then cooled in an ice-bath. The amine from preparation 28 (600 mg, 3 mmol) and N-methylmorpholine (0.6 ml, 5.45 mmol) were added and the reaction stirred at room temperature for 18 hours. The reaction mixture was concentrated under reduced pressure, and partitioned between water and ether. The organic layer was washed with hydrochloric acid (2N), sodium bicarbonate solution, then water, dried (MgSO₄) and evaporated under reduced pressure. The residual green solid was purified by medium pressure column chromatography on silica gel using ethyl acetate:hexane (90:10) as eluant to afford the title compound, 880 mg, 57%; ¹H NMR (CDCl₃, 300 MHz) δ: 1.37-2.28 (m, 12H), 2.46-2.64 (m, 1H), 3.20 (s, 3H), 3.31 (m, 2H), 4.97 (dd, 2H), 5.08 (dd, 2H), 6.57 (d, 1H), 7.12 (m, 1H), 7.18-7.48 (m, 10H), 8.08 (d, 1H).

Preparation 63

4-{[(1-{3-tert-Butoxy-2-[(2-methoxyethoxy)methyl]-3-oxopropyl}cyclopentyl)-carbonyl]amino}cyclohexanecarboxylic acid

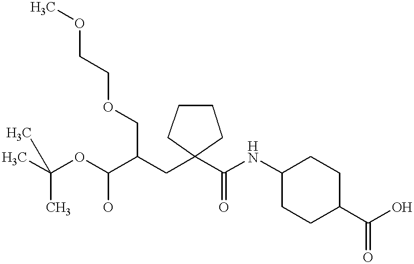

A mixture of benzyl 4-{[(1-{3-tert-butoxy-2-[(2-methoxyethoxy)methyl]-3-oxopropyl}cyclopentyl)carbonyl]amino}cyclohexanecarboxylate (EP 274234, Example 96), and 10% palladium on charcoal (250 mg) in water (10 ml) and ethanol (50 ml) was hydrogenated at 50 psi and room temperature for 18 hours. The reaction mixture was filtered through Solkafloc®, the filtrate concentrated under reduced pressure and the residue azeotroped with toluene (3×) and then dichloromethane (3×), to give the title compound, 2.0 g, 96%; ¹H NMR (CDCl₃, 300 MHz) δ: 1.48 (s, 9H), 1.53-1.84 (m, 14H), 1.94-2.10 (m, 5H), 2.60 (m, 2H), 3.40 (s, 3H), 3.41-3.63 (m, 5H), 3.96 (m,1H), 5.90 (bd, 1H).

Preparation 64

tert-Butyl 3-{1-[(cyclopentylamino)carbonyl]cyclopentyl}-2-[(2-methoxyethoxy)methyl]-propanoate

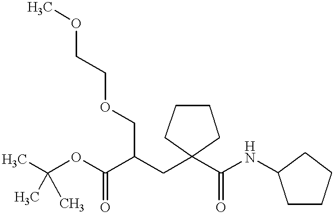

1-(3-Dimethylaminopropyl)-3-ethylcarbodiimide hydrochloride (197 mg, 1.07 mmol), 1-hydroxybenzotriazole hydrate (139 mg, 1.07 mmol), N-methylmorpholine (0.18 ml, 1.64 mmol) and cyclopentylamine (101 l, 1.07 mmol) were added to a solution of 1-{3-tert-butoxy-2-[(2-methoxyethoxy)methyl]-3-oxopropyl}-cyclopentanecarboxylic acid (EP 274234, Example 42) (400 mg, 1.07 mmol) in dichloromethane (5 ml), and the reaction stirred at room temperature for 22 hours. The reaction was quenched by the addition of water, extracted with dichloromethane (3×), and the combined organic extracts dried (MgSO₄) and evaporated under reduced pressure. The crude product was purified by column chromatography on silica gel using ethyl acetate:pentane (30:70) as eluant to afford the title compound as a clear oil, 320 mg, 78%; ¹H NMR (CDCl₃, 400 MHz) δ: 1.22-2.02 (m, 27H), 2.58 (m, 1H), 3.36 (s, 3H), 3.40 (m, 1H), 3.46 (m, 2H), 3.57 (m, 3H), 4.10-4.20 (m, 1H), 5.80 (bs, 1H).

Preparation 65

tert-Butyl 3-(2-methoxyethoxy)-2-{[1-({[3-(2-oxo-1-pyrrolidinyl)propyl]amino}carbonyl)cyclolentyl]methyl}propanoate

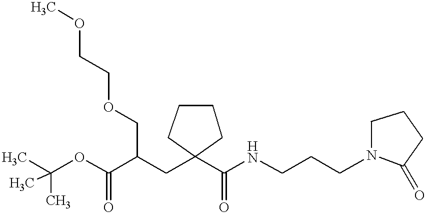

The title compound was obtained as a clear oil in 97% yield from 1-{3-tert-butoxy-2-[(2-methoxyethoxy)methyl]-3-oxopropyl}-cyclopentanecarboxylic acid (EP 274234, Example 42) and 1-(3-aminopropyl)-2-pyrrolidinone pyrrolidinone (Ex. Aldrich Chemical Co.), following a similar procedure to that described in preparation 64, except dichloromethane:methanol (95:5) was used as the column eluant, ¹H NMR (CDCl₃, 400 MHz) δ: 1.41 (s, 9H), 1.50 (m, 2H), 1.60-1.70 (m, 7H), 1.78 (m, 1H), 1.90 (m, 1H), 2.20 (m, 4H), 2.40 (m, 2H), 2.58 (m, 1H), 3.14 (m, 1H), 3.20 (m, 1H), 3.38 (m, 6H), 3.42-3.60 (m, 6H), 7.00 (m, 1H).

Preparation 66

cis-tert-Butyl 3-(2-methoxyethoxy)-2-[(1-{[(4-{[(phenylsulfonyl)amino]carbonyl}-cyclohexyl)amino]carbonyl}cyclopentyl)methyl]propanoate

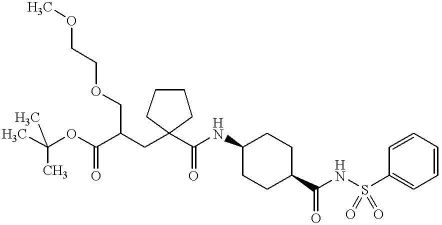

N,N′-Dicyclohexylcarbodiimide (199 mg, 0.97 mmol), 4-dimethylaminopyridine (118 mg, 0.97 mmol) and benzenesulphonamide (152 mg, 0.97 mmol) were added to an ice-cooled solution of the acid from preparation 63 (400 mg, 0.878 mmol) in dichloromethane (12 ml) and N,N-dimethylformamide (0.5 ml), and the reaction stirred at room temperature for 20 hours. The mixture was concentrated under reduced pressure and the residue suspended in cold ethyl acetate. The resulting insoluble material was filtered off, the filtrate washed with hydrochloric acid (1N), and water, then dried (MgSO₄) and evaporated under reduced pressure. The crude product was purified by column chromatography on silica gel using an elution gradient of dichloromethane:methanol (95:5 to 90:10) to afford the title compound as a white foam, 480 mg, 92%; ¹H NMR (CDCl₃, 400 MHz) δ: 1.44 (s, 9H), 1.63 (m, 13H), 1.80 (m, 2H), 1.88 (m, 1H), 1.98 (m, 2H), 2.36 (m, 1H), 2.57 (m, 1H), 3.38 (s, 3H), 3.40 (m, 1H), 3.51 (t, 2H), 3.58 (m, 3H), 3.95 (m, 1H), 5.92 (d, 1H), 7.56 (m, 2H), 7.62 (m, 1H), 8.05 (d, 2H), 8.75 (bs, 1H); LRMS: m/z 618 (MNa⁺).

Preparation 67

Benzyl 2-{[1-({[3-(2-Oxo-1-pyrrolidinyl)propyl]amino}carbonylcyclopentyl]-methyl}-4-phenylbutanoate

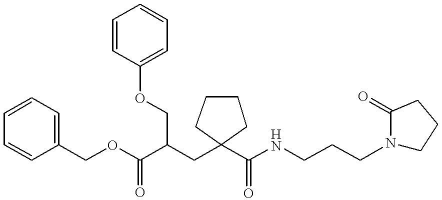

1-(3-Dimethylaminopropyl)-3-ethylcarbodiimide hydrochloride (1.06 g, 5.53 mmol), 1-hydroxybenzotriazole hydrate (0.60 g, 4.44 mmol) and 4-methylmorpholine (0.56 g, 5.54 mmol) were added sequentially to a cooled solution of 1-{2-[(benzyloxy)carbonyl]-4-phenylbutyl}cyclopentanecarboxylic acid (EP 274234 Example 17) (1.5 g, 3.94 mmol) in dry dichloromethane (15 ml) at room temperature, followed by N-(3-aminopropyl)-2-pyrrolidinone (Ex. Aldrich Chemical Co.) (0.56 g, 3.94 mmol), and the reaction stirred at room temperature for 18 hours. The mixture was washed with water, 2N hydrochloric acid, saturated aqueous sodium bicarbonate solution, and then dried (MgSO₄) and evaporated under reduced pressure. The residual yellow oil was purified by column chromatography on silica gel using ethyl acetate:pentane (50:50) as the eluant to provide the title compound as a clear gum, 800 mg, 40%; ¹H NMR (CDCl₃, 300 MHz) δ: 1.37-2.20 (m, 16H), 2.34-2.58 (m, 5H), 2.92-3.46 (m, 6H), 5.07 (d, 1H), 5.18 (d, 1H), 6.98-7.47 (m, 10H).

Preparation 68

Benzyl 2-{[1-({[3-(methylamino)-3-oxopropyl]amino}carbonyl)cyclopentyl]methyl}-4-phenylbutanoate

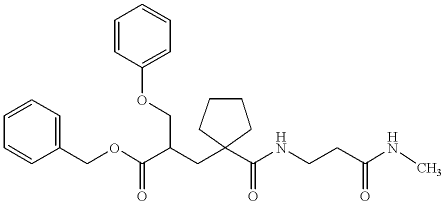

1-(3-Dimethylaminopropyl)-3-ethylcarbodiimide hydrochloride (122 mg, 0.64 mmol), 1-hydroxybenzotriazole hydrate (86 mg, 0.64 mmol) and 4-methylmorpholine (173 μl, 1.59 mmol) were added sequentially to a cooled solution of 1-{2-[(benzyloxy)carbonyl]-4-phenylbutyl}cyclopentane-carboxylic acid (EP 274234, Example 17) (202 mg, 0.53 mmol) in N,N-dimethylformamide (5 ml) at room temperature, followed by the amine hydrochloride from preparation 23 (146 mg, 1.06 mmol), and the reaction stirred at 90° C. for 18 hours. The cooled solution was concentrated under reduced pressure and the residue partitioned between water (20 ml) and ethyl acetate (100 ml). The layers were separated, the organic phase washed with water (3×30 ml), brine (25 ml) dried (MgSO₄), and evaporated under reduced pressure to give a clear oil. The crude product was purified by column chromatography on silica gel using dichloromethane:methanol (98:2) as eluant to afford the title compound as a colourless oil, 162 mg, 67%; ¹H NMR (CDCl₃, 400 MHz) δ: 1.38-1.53 (m, 2H), 1.53-1.96 (m, 8H), 2.02 (m, 2H), 2.27 (t, 2H), 2.46 (m, 3H), 2.76 (d, 3H), 3.44 (m, 2H), 5.13 (s, 2H), 5.79 (bs, 1H), 6.38 (m, 1H), 7.06 (d, 2H), 7.18 (m, 1H), 7.22 (m, 2H), 7.38 (m, 5H); LRMS: m/z465.5 (MH⁺).

Preparation 69

Benzyl 2-{[1-({[1-(hydroxymethyl)cyclopentyl]amino}carbonyl)cyclopentyl]methyl}-4-phenylbutanoate

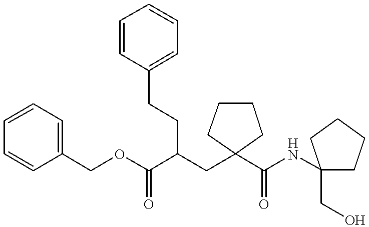

The title compound was obtained as a crystalline solid (48%) from 1-{2-[(benzyloxy)carbonyl]-4-phenyl butyl}cyclopentane-carboxylic acid (EP 274234, Example 17) and 1-amino-1-cyclopentanemethanol, following a similar procedure to that described in preparation 68, except the reaction mixture was stirred at room temperature for 18 hours, and the crude product purified by column chromatography on silica gel using ethyl acetate:pentane as eluant; ¹H NMR (CDCl₃, 400 MHz) δ: 1.38 (m, 2H), 1.50-1.95 (m, 16H), 2.01 (m, 2H), 2.45 (m, 3H), 3.49 (dd, 1H), 3.60 (dd, 1H), 4.58 (m, 1H), 5.10 (s, 2H), 5.67 (s, 1H), 7.01 (d, 2H), 7.14 (m, 1H), 7.20 (m, 2H), 7.36 (m, 5H); LRMS: m/z 478.3 (MH⁺).

Preparation 70

Benzyl 2-[(1-{[(5-methyl-1,3,4-thiadiazol-2-yl)amino]carbonyl}cyclopentyl)methyl]-4-phenylbutanoate

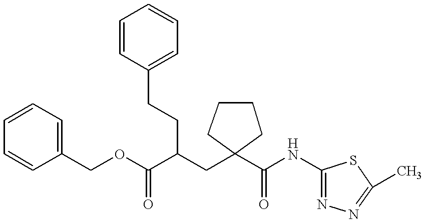

The title compound was obtained as a clear oil in 74% yield from 1-{2-[(benzyloxy)carbonyl]-4-phenylbutyl}cyclopentane-carboxylic acid (EP 274234, Example 17) and 2-amino-5-methyl-1,3,4-thiadiazole (ex Lancaster), following a similar procedure to that described in preparation 68; ¹H NMR (CDCl₃, 400 MHz) δ: 1.58-1.76 (m, 7H), 1.83-1.98 (m, 3H), 2.03 (m, 1H), 2.20 (m, 1H), 2.35 (m, 1H), 2.44 (m, 3H), 2.65 (s, 3H), 5.02 (dd, 2H), 7.00 (d, 2H), 7.15 (m, 1H), 7.19 (m, 2H), 7.35 (m, 5H); LRMS: m/z 478.7 (MH⁺).

Preparation 71

Benzyl 4-phenyl-2-({1-[(3-pyridinylamino)carbonyl]cyclopentyl}methyl)butanoate

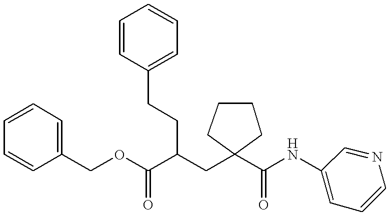

Oxalyl chloride (2.29 ml, 26.3 mmol) was added to a solution of 1-{2-[(benzyloxy)carbonyl]-4-phenylbutyl}cyclopentane-carboxylic acid (EP 274234, Example 17) (5.0 g, 13.14 mmol) and N,N-dimethylformamide (2 drops) in dichloromethane (25 ml), and the solution stirred for 2.5 hours. The mixture was evaporated under reduced pressure, the residue azeotroped with dichloromethane to give a yellow oil. This was then dissolved in dichloromethane (50 ml) and a solution of this acid chloride (10 ml, 2.45 mmol) was added to an ice-cooled solution of triethylamine (248 mg, 2.45 mmol) and 3-aminopyridine (253 mg, 2.70 mmol) in dry dichloromethane (10 ml), and the reaction stirred at room temperature for 18 hours. The solution was washed with water (3×), dried (MgSO₄) and evaporated under reduced pressure. The crude product was purified by column chromatography on silica gel using ethyl acetate:hexane (40:60) as eluant, and repeated using an elution gradient of ether:hexane (90:10 to 100:0). The product was crystallised from ethyl acetate:hexane to afford the title compound, 740 mg, 66%; ¹H NMR (CDCl₃, 300 MHz) δ: 1.38-2.07 (m, 10H), 2.10-2.37 (m, 2H), 2.42-2.63 (m, 3H), 5.02 (s, 2H), 6.94-7.44 (m, 10H), 7.50 (s, 1H), 8.03 (d, 1H), 8.36 (d, 1H), 8.52 (s, 1H).

Preparation 72

trans-tert-Butyl-3-[1-({[2-(4-chlorophenyl)cyclopropyl]amino}carbonyl)-cyclopentyl]-2-(methoxymethyl)propanoate

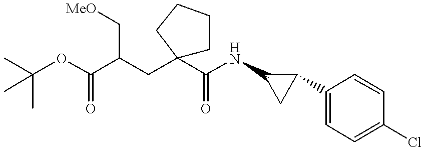

The product from Preparation 94 (286 mg, 1 mmol), the product from Preparation 76 (203 mg, 1 mmol), 1-(3-dimethylaminopropyl)-3-ethylcarbodiimide (211 mg, 1.1 mmol), triethylamine (1 ml) and HOBt (148 mg, 1.1 mmol) in DCM (5 ml) were stirred at room temperature for 16 h. The reaction mixture was washed with water (20 mls), dried over MgSO₄ and purified by column chromatography using 1:8, then 1:5 EtOAc:pentane as eluant to provide the title product as a colourless film (122 mg, 40%); R_{f} 1:5 (EtOAc:pentane) 0.2; ¹HNMR (400 MHz, CDCl₃) δ: 1.05-1.2 (m, 2H), 1.4 (s, 9H), 1.55-1.75 (m, 4H), 1.9-2.0 (m, 4H), 2.4-2.5 (m, 1H), 2.75-2.85 (m, 1H), 3.3 (s, 3H), 3.4-3.5 (m, 1H), 6.3 (m, 1H), 7.05 (d, 2H), 7.2 (d, 2H); HRMS: m/z M+H, Found 436.2242. C₂₄H₃₅NO₄Cl requires 436.2249.

Preparation 73

Ethyl-2-(4-chlorophenyl)cyclopropanecarboxylate

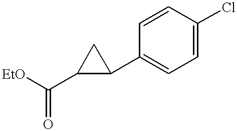

A mixture of 4-chlorostyrene (10.1 ml, 96 mmol) and rhodium acetate dimer (1 g, 4.5 mmol) in toluene (50 ml) was heated to 85° C. before adding ethyl diazoacetate (11.3 mls, 94 mmol) over 30 mins and the whole then heated at 80° C. for a further 1 h before concentration in vacuo. The residue was then purified by column chromatography using 1:2 DCM:pentane as eluant to give the title product as a colourless oil (7.8 g, 37%); R_{f} 1:2 (DCM:pentane) 0.35; ¹HNMR (400 MHz, CDCl₃) δ 1.15-1.3 (m, 4H), 1.5-1.7 (m, 1H), 1.8-1.9 (m, 1H), 2.4-3.55 (m, 1H), 4.2 (q, 2H), 6.95 (d, 2H), 7.20-7.28 (m, 2H); and LRMS: m/z, M+NH₄+242.

Preparation 74

trans-2-(4-Chlorophenyl)cyclopropanecarboxylic acid

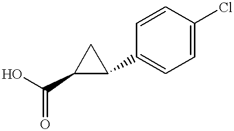

The product from preparation 73 (7.8 g, 37 mmol) was dissolved in EtOH (75 mls) at room temperature under nitrogen and sodium methoxide (8.1 g, 150 mmol) was added portionwise over 15 mins. After the addition was complete, the mixture was then refluxed for 18 h. The reaction mixture was concentrated in vacuo, and the resulting residue diluted with DCM and water (150 mls, 2:1 mixture). The organic layer was removed, and the aqueous layer re-extracted with DCM (2×50 mls). The combined organic extracts were dried over MgSO₄ and evaporated to provide the trans ester (4.96 g, 62%). Acidification of the aqueous layer with concentrated HCl to pH 1 resulted in a white precipitate, which was filtered and dried under vacuum to provide the hydrolysis product (the corresponding acid) as a white powder (1 .95 g, 27%). Dissolution of the ester in MeOH (50 mls), water (50 mls) and LiOH (1.34 g, 32 mmol) gave a clear solution which was heated at ca. 70° C. overnight. The reaction mixture was cooled, concentrated in vacuo, and acidified with concentrated HCl to pH 1. The resulting white precipitate was extracted with EtOAc (3×50 mls) and the combined organic extracts were dried over MgSO₄ and evaporated to dryness, to provide the acid (4 g, 96%). This acid was combined with the hydrolysed product from the previous step to give a total of 5.95 g; ¹HNMR (400 MHz, CDCl₃) δ 1.3-1.4 (m, 1H), 1.6-1.7 (m, 1H), 1.8-1.9 (m, 1H), 2.5-2.6 (m, 1H), 7.00 (d, 2H), 7.26 (d, 2H); LRMS: m/z, M-H 195.

Preparation 75

trans-tert-Butyl-2-(4-chlorophenyl)cyclopropylcarbamate

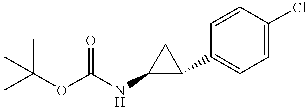

The product from preparation 74 (1.5 g, 7.65 mmol), DPPA (1.8 ml, 8.4 mmol) and triethylamine (1.25 ml, 12 mmol) in tert-butanol (20 ml) was heated at ca. 90° C. under nitrogen for 48 h. When cool, the mixture was diluted with EtOAc (20 mls) and saturated Na₂CO₃ solution (20 mls) and the organic layer was then removed. The aqueous layer was reextracted with EtOAc (20 mls) and the combined organic layers were dried over MgSO₄, filtered and evaporated. The resulting residue was purified by column chromatography using 1:10, then 1:2 EtOAc:pentane as eluant to provide the title product as a white solid (1.7 g, 83%); R_{f} 1:2 (EtOAc:pentane) 0.9; ¹HNMR (400 MHz, CDCl₃) δ: 1.2-1.3 (m, 1H), 1.4 (s, 10H), 1.9-2.0 (m, 1H), 2.6 (br.s, 1H), 4.8 (br.s, 1H), 7.0 (d, 2H), 7.2 (d, 2H); HRMS: m/z M+Na, Found 290.0923. C₁₄H₁₈NO₂ClNa requires 290.0918.

Preparation 76

2-(4-Chlorophenyl)cyclopropylamine

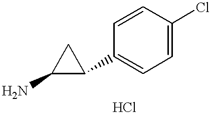

The product from Preparation 75 was taken up in EtOAc, cooled to 0° C. and hydrogen chloride gas was bubbled through the solution for 30 mins. The solution was then concentrated in vacuo to give the title product (1.29 g, 6.3 mmol); R_{f} 1:3 (EtOAc:pentane) 0; ¹HNMR (400 MHz, CD₃OD) δ: 1.3-1.4 (m, 1H), 1.4-1.5 (m, 1H), 2.3-2.4 (m, 1H), 2.8-2.9 (m, 1H), 7.15 (d, 2H), 7.3 (d, 2H).

Preparation 77

Ethyl-2-(4-methoxyphenyl)-cyclopropanecarboxylate

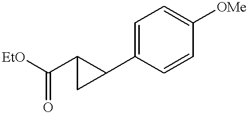

A mixture of 4-methoxystyrene (25.5 mls, 192 mmols), rhodium acetate dimer (2 g, 4.5 mmol) and toluene (100 mls) was stirred at room temperature under nitrogen for 20 mins and then heated to 85° C. Ethyl diazoacetate (19.8 mls, 188 mmols) was added dropwise over 50 mins at a rate of one drop every 2 to 3 seconds to maintain the internal reaction temperature at around 95° C. After the addition was complete, the mixture was heated for 1 h at 85° C. and then cooled to room temperature. The mixture was filtered through Arbocel® and evaporated to an oil which was purified by column chromatography using DCM:pentane (1:2) as eluant to provide the title product (1 3 g, 31%) which was a 3:1 mixture of the trans:cis isomers; R_{f} 0.22 (DCM:pentane) 1:2; ¹HNMR (400 MHz, CDCl₃) δ: 1.20-1.38 (m, 5H), 1.83 (ddd, 1H), 2.50 (ddd, 1H), 3.80 (s, 3H), 4.16 (q, 2H), 6.82 (d, 2H), 7.03 (d, 2H).

Preparation 78

Trans-2-(4-methoxyphenyl)-cyclopropanecarboxylic acid

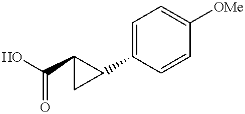

Sodium methoxide (14.8 g, 273.8 mmol) was added to a solution of the product from Preparation 77 (13 g, 59 mmol) in EtOH (135 mls) while stirring at room temperature under a nitrogen atmosphere. After the addition was complete, the mixture was refluxed gently for 1 h, by which time TLC analysis indicated that there was no cis isomer remaining. The reaction was cooled to room temperature and water (100 mls) added in one portion. The whole was then stirred at room temperature for 63 h and then evaporated to remove the MeOH before acidifying with concentrated HCl to pH 1. The suspension was extracted with EtOAc (2×100 mls), and the extracts dried (MgSO₄) and evaporated to give a yellow solid, which was purified by column chromatography using 1:1 EtOAc:pentane to provide the title product (8.9 g, 78%); R_{f} 1:1 (EtOAc:pentane) 0.4; ¹HNMR (400 MHz, CDCl₃) δ: 1.30-1.41 (m, 1H), 1.58-1.69 (m, 1H), 1.79-1.90 (m, 1H), 2.53-2.62 (m, 1H), 6.83 (d, 2H), 7.04 (d, 2H).

Preparation 79

Trans-tert-Butyl-2-(4-methoxyphenyl)-cyclopropylcarbamate

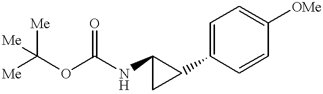

DPPA (11 mls, 50.9 mmol) was added to a stirred mixture of the product from Preparation 78 (8.9 g, 46.3 mmol), triethylamine (10.1 mls, 72.7 mmol) and tert-BuOH (75 mls). The mixture was heated at 90° C. for 43 h. When cool, the tert-BuOH was removed by evaporation and the resulting oily residue treated with saturated K₂CO₃ (120 ml) and then extracted with EtOAc (2×100 mls). The combined organic extracts were then evaporated under reduced pressure to give a brown solid which was purified by column chromatography using DCM:MeOH (98:2) as eluant to provide the title product (5.8 g, 148%); ¹HNMR (400 MHz, CDCl₃) δ: 1.07-1.14 (m, 2H), 1.44 (s, 9H), 1.93-2.06 (m, 1H), 2.62-2.71 (m, 1H), 3.80 (s, 3H), 4.72-4.88 (m, 1H), 6.80 (d, 2H), 7.08 (d, 2H).

Preparation 80

Trans-2-(4-methoxyphenyl)-cyclopropylamine

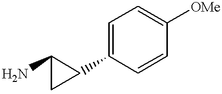

TFA (20 mls) was added to a stirred mixture of the product from Preparation 79 (5.8 g, 22.0 mmol) and DCM (I5 mls) at room temperature under nitrogen. The reaction was stirred for 16 h, after which time the solvent was removed under reduced pressure, and the resulting oil treated with saturated aqueous K₂CO₃ until a pH of 10 was reached (ca. 150 mls required). This opaque solution was extracted with EtOAc (2×150 mls) and the extracts were then dried over MgSO₄ and evaporated to give a biege solid. This solid was purified by column chromatography using 99:1, the 95:5 DCM:MeOH as eluant to give the title product as a white solid (3.2 g, 89%); R_{f} DCM:MeOH (19:1)0.18; ¹HNMR (400 MHz, CDCl₃) δ: 0.87-1.04 (m, 2H), 1.79-1.90 (m, 1H), 2.43-2.54 (m, 1H), 3.80 (s, 3H), 6.80 (d, 2H), 6.98 (d, 2H).

Preparation 81

Tert-Butyl-4-methoxy-2-{[1-({[2-(4-methoxyphenyl)cyclopropyl]amino}carbonyl)-cyclopentyl]methyl}butanoate

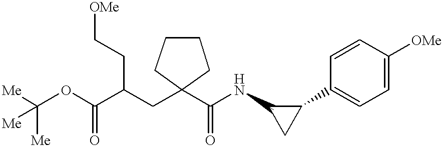

The product from Preparation 99 (210 mg, 0.66 mmol), triethylamine (1 ml), HOBt (140 mg, 0.73 mmol) and the product from Preparation 80 (107 mg, 0.66 mmol) were combined sequentially in DCM at room temperature under nitrogen. WSCDI (98 mg, 0.73 mmol) was then added to the mixture and the whole stirred overnight for some 16 h. The reaction was diluted with water, the organic layer was separated and then washed with brine, dried over MgSO₄, filtered and evaporated to a yellow oil. This was purified by column chromatography using 1:3 EtOAc:pentane as eluant to provide the title product (113 mg, 38%); R_{f} 1:10 (EtOAc:pentane) 0.2; ¹NMR (400 MHz, CDCl₃) δ: 1.0-1.10 (m, 2H), 1.40 (s, 9H), 1.35-1.45 (m, 2H), 1.50-1.80 (m, 7H), 1.85-2.10 (m, 4H), 2.25-2.35 (m, 1H), 2.70-2.80 (m, 1H), 3.2 (s, 3H), 3.25-3.35 (m, 2H), 3.7 (s, 3H), 6.05 (br.s, 1H), 6.70 (d, 2H), 7.05 (d, 2H); LRMS: m/z 446 (M+H).

Preparation 81a

Tert-Butyl-4-methoxy-2-{[1-({[2-phenylcycloproryl]amino}carbonyl)-cyclopentyl]methyl}butanoate

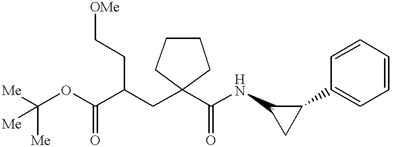

The title product was prepared in analogous fashion to Preparation 81 replacing the product from Preparation 80 with 1-S-amino-2-R-phenyl cyclopropane (J. Med. Chem., 1986, 29, 2044); ¹NMR (400 MHz, CDCl₃) δ: 1.1 (m, 1H), 1.2 (m, 1H), 1.4 (s, 9H), 1.6 (m, 8H), 1.7, (m, 1H), 1.9 (m, 2H), 2.1 (m, 2H), 2.4 (q, 1H), 2.8 (q, 1H), 3.2 (s, 3H), 3.3 (q, 2H), 6.1 (bs, 1H), 7.1 (d, 3H), 7.2 (d, 2H). ; LRMS: m/z416 (M+H); HRMS m/z Found 416.2794. C₂₅H₃₂NO₄ requires 416.2796.

Preparation 82

Tert-Butyl-3-methoxy-2-[(1-{[(2-phenylcyclopropyl)amino]carbonyl}cyclopentyl)-methyl]propanoate

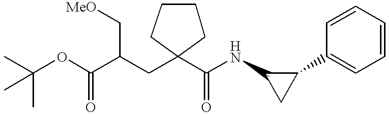

The product from Preparation 94 (200 mg, 0.66 mmol), triethylamine (1 ml), HOBt (98 mg, 0.73 mmol) and 1-S-amino-2-R-phenyl cyclopropane (J. Med. Chem., 1986, 29, 2044) (123 mg, 0.73 mmol) were combined sequentially in DCM (6 mls) at room temperature under nitrogen. WSCDI (98 mg, 0.73 mmol) was then added to the mixture and the whole stirred overnight for some 16 h. The reaction was diluted with water, the organic layer was separated and then washed with brine, dried over MgSO₄, filtered and evaporated to a yellow oil. This was purified by column chromatography using 1:3 EtOAc:pentane as eluant to provide the title product (164 mg, 62%); R_{f} 1:10 (EtOAc:pentane) 0.2; ¹HNMR (400 MHz, CDCl₃) δ: 1.0-1.1 (m, 2H), 1.4 (s, 9H), 1.5-1.8 (m, 6H), 1.8-2.05 (m, 4H), 2.3-2.4 (m, 1H), 2.7-2.8 (m, 1H), 3.2 (s, 3H), 3.25-3.35 (m, 1H), 3.7 (s, 3H), 6.05 (s, 1H), 6.7 (d, 2H), 7.05 (d, 2H); LRMS: m/z M+H, 446.

Preparation 83

3-Phenyl cyclopentanone

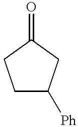

Phenyl magnesium bromide (0.27 moles) was taken up in dry diethyl ether (200 ml) and cooled to 0° C. under a nitrogen atmosphere. Copper (I) iodide (25.5 g, 0.13 moles) was added in one portion, and the suspension stirred at 0° C. for 20 mins. Cyclopenten-2-one was then added dropwise over 10-15 mins and the resulting solution stirred at 0° C. for 10 mins, and then allowed to warm to room temperature over the course of 1 h. The reaction mixture was added to 100 mls of a mixture of saturated ammonium chloride solution and concentrated ammonia, the pH of which was initially measured at 9. The whole was stirred at room temperature for 30 mins, and then filtered, and the layers of the filtrate were then separated. The aqueous layer was extracted with ether (2×70 mls) and the extracts combined with the original organic layer. The bulked ether layers were then washed with brine, dried over MgSO₄, filtered and evaporated to a pale yellow oil. This oil was then chromatographed using 1:3 ether/pentane to give the title product (2.9 g, 14%); R_{f} EtOAc:pentane (1:2) 0.65; ¹HNMR (300 MHz, CDCl₃) δ: 1.91-2.10 (m, 1H), 2.18-2.59 (m, 4H), 2.67 (dd, 1H), 3.38-3.52 (m, 1H), 7.19-7.47 (m, 5H).

Preparation 84

7-Phenyl-1 3-diazaspiro[4.4]nonane-2,4-dione

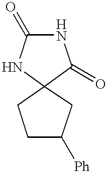

The product from Preparation 83 (5.8 g), potassium cyanide (2.75 g) and ammonium carbonate (9.1 g) were heated in 80 mls of 50% aqueous EtOH for 7 h, and then at room temperature for 48 h. The mixture was filtered, and the solid washed thoroughly with water (3×50 mls). The filtrate was concentrated, the pH adjusted to 2 using concentrated HCl and the resulting suspension filtered off and washed with water (3×50 mls). The bulked solids were recrystallised from EtOH and water (300 ml:100 ml) to give the title product as 1:1 mixture of diatereoisomeric pairs (6.32 g); R_{f} 0.6 in EtOAc; ¹HNMR (300 MHz, CDCl₃) δ: 1.60-2.58 (m, 6H), 3.08-3.37 (m, 1H), 6.83-7.44 (m, 5H), 8.33 (d, 1H), 10.60 (s, 1H);

Anal. Found: C, 68.09; H, 6.21; N, 12.25%. C₁₃H₁₄N₂O₂ requires C, 67.81; H, 6.13; N, 12.17%.

Preparation 85

1-Amino-3-phenyl-cyclopentane carboxylic acid

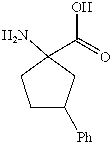

The title product from Preparation 84 (6.2 g), barium hydroxide octahydrate (17.2 g) and water (100 ml) were heated together in a bomb at 160° C. for 7 h, and then allowed to stand overnight at room temperature. The reaction mixture was acidified to pH 1 using concentrated H₂SO₄. The resulting suspension was then filtered and the solid was washed with water (100 mls). The filtrate was then basified to ca. pH 6 using concentrated ammonia solution, the suspension cooled in a ice bath, and then filtered. The solid was washed with water, and dried under vacuum to give the title product (2.9 g); m.p. 265° C. (dec.); R_{f} 0.5 in methyl isobutyl ketone: acetic acid: water (2:1:1); ¹HNMR (300 MHz, CDCl₃) δ: 2.06-3.14 (m, 6H), 3.42-3.73 (m, 1H), 7.12-7.44 (m, 5H);

Anal. Found C, 69.54; H, 7.26; N, 6.73%. C₁₂H₁₅NO₂ requires C, 70.22; H, 7.37; N, 6.82%.

Preparation 86

Ethyl-1-amino-3-phenyl-cyclopentanecarboxylate

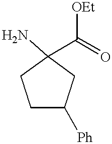

The title product from Preparation 85 (500 mg, 2.4 mmol) was taken up in EtOH saturated with hydrogen chloride (70 ml) at 0° C. and then stirred at room temperature for 16 h. Nitrogen gas was then bubbled through the solution for 10 mins, and the solvent evaporated to give a biege solid. This was treated with saturated aq. NaHCO₃ solution (10 mls) and extracted with EtOAc (2×10 mls). The combined organic extracts were dried and evaporated to give the title product (360 mg, 63%); R_{f} DCM:MeOH (97:3) 0.23; ¹HNMR (400 MHz, CDCl₃) δ: 1.31 (t, 3H), 1.50-2.37 (m, 4H), 2.38-2.44 (m, 1H), 2.63 (dd, 1H), 3.22-3.36 and 3.43-3.57 (m, 1H), 4.20 (q, 2H), 7.20-7.35 (m, 5H).

Preparation 87

(1-Amino-3-phenylcyclopentyl)methanol

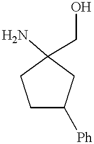

Sodium borohydride (1 90 mg) was added portionwise to a stirred solution of the product from Preparation 86 (390 mg, 1.67 mmol) in 8 mls of a 50% solution of aqueous EtOH and then heated at 50° C. for 3 h. The reaction mixture was then evaporated to give a suspension of an oil in water. This oil was extracted with EtOAc (20 mls), and evaporated under reduced pressure to provide the title product (295 mg); R_{f} (1:2 ether:pentane) 0.65; ¹HNMR (400 MHz, CDCl₃) δ: 1.30-2.36 (m, 6H), 3.02-3.17 (m, 1H), 3.33-3.50 (m, 2H), 7.14-7.37 (m, 5H).

Preparation 88

Tert-Butyl-2-{[1-({[1-(hydroxymethyl)-3-phenylcyclopentyl]amino}carbonyl)-cyclopentyl]methyl}-4-methoxybutanoate

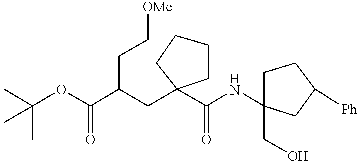

The title product was prepared by a similar method to that described in preparation 33 from the product from Preparation 99 and the title product from preparation 87; R_{f} EtOAc: pentane (1:4) 0.25; ¹HNMR (400 MHz, CDCl₃) δ: 0.80-0.88 (m, 3H), 1.16-2.60 (m, 21H), 1.42 (s, 9H), 3.04-3.32 (m, 1H), 3.57-3.84 (m, 2H), 4.56-4.77 (m, 1H), 5.94 (br.t, 1H), 7.16-7.28 (m, 5H).

Preparation 89

Tert-Butyl-4-methoxy-2-[(1-{[(2-pentylcyclopropyl)amino]carbonyl}cyclopentyl)-methyl]butanoate

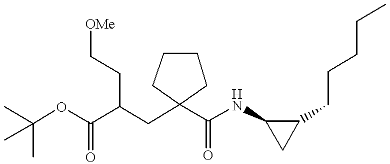

The title product from preparation 99 (105 mg, 0.33 mmol), triethylamine (0.5 ml), HOBt (49 mg, 0.36 mmol) and 1-amino-2-pentyl-cyclopropane (100 mg, 0.33 mmol) were combined sequentially in DCM at room temperature under nitrogen. WSCDI (70 mg, 0.36 mmol) was then added to the mixture and the mixture stirred overnight for 16 h. The reaction was diluted with water, the organic layer was separated and then washed with brine, dried over MgSO₄, filtered and evaporated to a yellow oil. This was purified by column chromatography using 1:3 EtOAc:pentane as eluant to give the title product (96 mg, 71%); R_{f} 1:6 (EtOAc:pentane) 0.2; ¹H NMR (400 MHz, CDCl₃) δ: 0.4-0.6 (m, 2H), 0.7-0.9 (m, 4H), 1.05-1.15 (m, 1H), 1.2-1.3 (m, 4H), 1.3-1.5 (m, 14H), 1.5-2.0 (m, 1OH), 2.2-2.4 (m, 2H), 3.2-3.35 (m, 5H), 5.7-5.9 (br.s, 1H); LRMS: m/z, M+H, 410.

Preparation 90

4-Butylpyridine

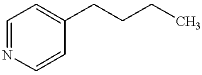

Lithium diisopropylamide was formed by the addition n-butyllithium (43 ml of a 2.5M solution in hexanes) to a stirred solution of diisopropylamine (1 0.9 g) in dry THF (1 00 mls) at −30° C. under nitrogen. After 1 h of stirring at this temperature, the solution was cooled to −78° C. and a solution of 4-methyl pyridine was added (10 g) in dry THF (20 ml), followed by continued stirring at −78° C. for 1 h. Iodopropane (20 g) was added dropwise over 45 mins as a solution in 20 mls dry THF, followed by continued stirring of the whole mixture for 1 h. Saturated aqueous ammonium chloride solution (20 ml) was added, and the reaction was then extracted with ether (2×100 mls). The combined ether extracts were washed with water (75 mls), dried over MgSO₄ and evaporated to give a clear oil. This oil was purified by distillation under water aspiration vacuum (ca. 30 mmHg) and the title product was collected as the fraction which distilled over at 84-90° C.; ¹H NMR (400 MHz, CDCl₃) δ: 0.93 (t, 3H, Me), 1.30-1.42 (m, 2H), 1.57-1.66 (m, 2H), 2.60 (t, 2H), 7.06 (d, 2H), 8.46 (d, 2H).

Preparation 90a

2-Amino-4-butyl pyridine

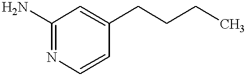

The title product was prepared from the product from Preparation 90 according to the method described in the Journal of the Chemical Society, 1946, p936.

Preparation 91

Benzyl-2-[(1-{[(4-butyl-2-pyridinyl)-amino]carbonyl}cyclopentyl)methyl]-4-methoxy-butanoate

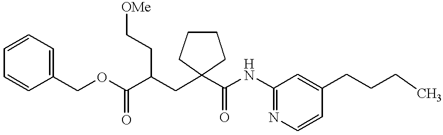

The title product was prepared by a similar procedure to that described in preparation 62 from 1-{2-[(benzyloxy)carbonyl]-4-methoxybutyl}-cyclopentanecarboxylic acid (EP 274234, Example 15) and the product from preparation 90a; ¹HNMR (CDCl₃, 400 MHz) δ: 0.89 (t, 3H, Me), 1.36 (q, 2H, CH₂), 1,47-2.26 (m, 14H), 2.55-2.68 (m, 3H), 3.17 (s, 3H, OMe), 3.24 (t, 2H, CH₂OMe), 4.91 (d, 1H, CHPh), 5.00 (d, 1H, CHPh), 6.83 (d, 1H, Ar), 7.27-7.35 (m, 5H), 7.94 (brs, 1H, NH), 8.07 (s, 1H, Ar), 8.13 (d, 1H, Ar).

Preparation 92

Benzyl-2-[(1-{[(4-phenyl-2-pyridinyl)-amino]carbonyl}cyclopentyl)methyl]-4-methoxy-butanoate

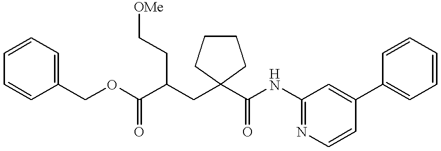

The title product was prepared by a similar procedure to that described in preparation 62 from 1-{2-[(benzyloxy)carbonyl]-4-methoxybutyl}-cyclopentanecarboxylic acid (EP 274234, Example 15) and 2-amino-4-phenyl pyridine (see Journal of Medicinal Chemistry, 1978, p874); ¹HNMR (CDCl₃, 400 MHz) δ: 1.43-2.34 (m, 10H), 2.60-2.68 (m, 1H), 3.17 (s, 3H, OMe), 3.26 (t, 2H, CH₂), 4.93 (d, 1H, CHPh), 5.02 (d, 1H, CHPh), 7.18-7.32 (m, 5H, Ph), 7.38-7.46 (m, 3H), 7.61-7.69 (m, 2H), 8.02 (brs, 1H, NH), 8.29 (d, 1H, Ar), 8.57 (s, 1H, Ar).

Preparation 93

Tert-Butyl-2-{[1-({[2-(hydroxymethyl)-2,3-dihydro- 1H-inden-2-yl]amino}carbonyl)-cyclopentyl]methyl}-4-methoxybutanoate

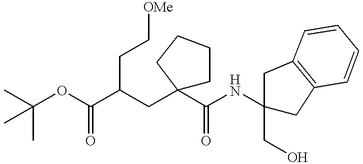

The title product was prepared by a similar procedure to that described in preparation 33 from the product from preparation 99 and and 2-amino-2-hydroxymethyl-2,3-dihydroindene (WO9110644; Example 8a); ¹HNMR (CDCl₃, 400 MHz) δ: 1.40 (s, 9H), 1.44-2.00 (m, 12H), 2.37-2.43 (m, 1H), 2.99 (d, 1H), 3.08 (d, 1H), 3.20-3.38 (m, 7H), 3.65 (dd, 1H), 3.84 (dd, 1H), 4.40 (t, 1H), 6.00 (s, 1H), 7.10-7.18 (m, 4H).

Preparation 94

3-(1-Carboxycyclopentyl)-2-(methoxymethyl)propanoic acid tert-butyl ester

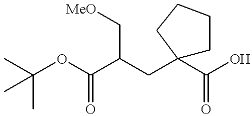

The title product from Preparation 2, stage b) (10 g, 41.3 mmol) was taken up in THF at −78° C. and lithium diisopropylamide (43 ml, 86.7 mmol, 2M solution in THF) added dropwise. The mixture was stirred at −78° C. for 40 min, after which time chloromethyl methyl ether (4.7 ml, 62 mmol) was added dropwise. The solution was then allowed to warm slowly to room temperature overnight and was quenched by the addition of 2N HCl (100 ml). The organics were extracted with EtOAc (2×100 ml), dried (MgSO₄) and purified by column chromatography using 2%, then 3%, and then 5% MeOH in DCM to provide the title product as a yellow oil (6.2 g, 53%); ¹HNMR (CDCl₃, 400 MHz) 6 1.40 (9H, s), 1.40-1.50 (4H, m), 1.20-1.80 (1H, m), 1.80-1.90 (1H, m), 2.00 (1H, dd), 2.00-2.05 (3H, m), 2.20 (1H, dd), 2.50-2.60 (1H, m), 3.30 (1H, s), 3.30-3.40 (1H, m), 3.40 (1H, t); LRMS: m/z, MNH₄ ⁺ 304.

Preparation 95

Tert-Butyl-2-[(1-{[(5-benzyl-1,3,4-thiadiazol-2-yl)amino]carbonyl}cyclopentyl)methyl]-4-methoxybutanoate

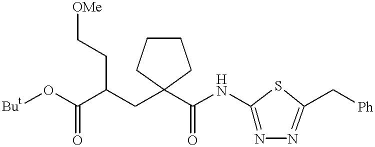

The product from Preparation 98 was reacted with the title product from Preparation 99 using a similar procedure to that described in Preparation 33 to provide the amide as a white foam; ¹HNMR (CDCl₃, 400 MHz) δ 0.80 (t, 3H), 1.20-1.35 (m, 2H), 1.37 (s, 9H), 1.40-2.20 (m, 13H), 4.30 (s, 2H), 7.30 (m, 5H); LRMS m/z 459 (M+H).

Preparation 96

Tert-Butyl-2-[(1-{[(2,3-dihydro-1-benzofuran-2-ylmethyl)amino]carbonyl}cyclopentyl)methyl]-4-methoxybutanoate

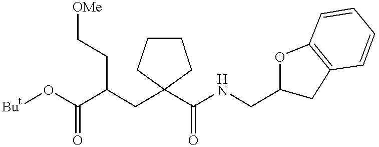

The title compound was prepared from 2-aminomethyl-2,3-dihydrobenzofuran (J. Med. Chem., 1968, 11(4), page 844) and the title product from Preparation 99 using a similar procedure to that described in Preparation 33; ¹HNMR (CDCl₃, 400 MHz) 6 1.4 (s, 10H), 1.45-2.00 (m, 12H), 2.05 (m, 1H), 2.30 (m, 1H), 2.90 (m, 1H), 3.05 (m, 5H), 3.30 (m, 2H), 3.55-3.65 (m, 1H), 4.80 (m, 1H), 6.15 (m, 1H), 6.70 (d, 1H), 6.80 (t, 1H), 7.00 (d, 1H), 7.05 (m, 1H); LRMS m/z 432 (M+H).

Preparation 97

5-(Ethoxymethyl)-1,3,4-thiadiazol-2-amine

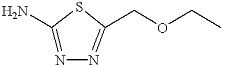

Prepared following a similar procedure to preparation 31 from ethoxyacetic acid.

Preparation 98

5-Benzyl-1,3,4-thiadiazol-2-amine

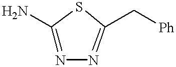

The title compound was prepared following a similar procedure to preparation 31 from phenyl acetic acid; ¹HNMR (CDCl₃, 400 MHz) δ 4.10 (m, 2H), 7.30 (m, 5H);

Anal. Found C, 56.73; H, 4.72; N, 21.67%. C₉H₉N₃S requires C, 56.62; H, 4.74; N, 21.97%.

Preparation 99

1-[2-(tert-Butoxycarbonyl)-4-methoxybutyl]cyclopentanecarboxylic acid

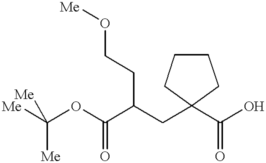

A solution of the title product from Preparation 2, stage b) in dry tetrahydrofuran (100 ml) was added to a stirred solution of lithium diisopropylamide (130 ml) in a mixture of hexane (52 ml) and tetrahydrofuran (200 ml) at −78° C. under nitrogen. After 1 hour a solution of 2-bromoethyl methyl ether in tetrahydrofuran (100 ml) was added maintaining the temperature at −78° C. The reaction mixture was allowed to warm up to room temperature overnight. The mixture was quenched with water (100 ml) and acidified to pH 1 with 2M hydrochloric acid, and extracted with ethyl acetate (2×150 ml). The combined organic extracts were dried over magnessium sulphate and concentrated in vacuo to give the crude acid which was chromatographed on silica. Elution with increasing proportions of methanol in dichloromethane (neat dichloromethane to 1:50) gave an oil (7.7 g, 25.6 mmol, 52%). R_{f} 0.3 methanol, dichloromethane 1:20. ¹H NMR (CDCl₃ 400 MHz) δ: 1.4 (s, 9H), 1.4-1.7 (m, 7H), 1.75-1.95 (m, 2H), 2.0-2.15 (m, 3H), 2.3-2.4 (m, 1H), 3.3 (s, 3H), 3.3-3.4 (m, 2H). LRMS: m/z 299 (M−H⁺).

Preparation 100

Benzyl-1-[2-(tert-butoxycarbonyl)pentyl]cyclopentanecarboxylate

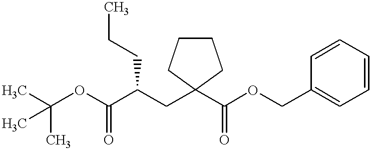

The product from Preparation 2 (513 mg, 1.80 mmol) was dissolved in methanol (5 mls of 75% aqueous methanol) and cesium carbonate (300 mg, 0.95 mmol) was added in one portion at room temperature. After 5 mins, the solvents were removed under reduced pressure, and the residue azeotroped with toluene (2×5 mls) and then redissolved in 7 mls of dry DMF under a nitrogen atmosphere. Benzyl bromide was taken up in 3 mls of dry DMF, and added slowly with stirring, before the reaction mixture was stirred at room temperature for 3 h. The mixture was poured into ethyl acetate (40 mls) and washed with water (40 mls), 1N HCl (20 mls) and water (2×20 mls). The organic layer was dried (MgSO₄) and evaporated to a thick oil, which was purified by column chromatography using 1:2 DCM:pentane, then 1:2 EtOAc:pentane as eluant to provide the title product (430 mg, 64%); ¹HNMR (400 MHz, CDCl₃) 0.83 (t, 3H), 1.17-1.32 (m, 3H), 1.42 (s, 9H), 1.36-1.68 (m, 7H), 1.80 (dd, 1H), 1.97-2.12 (m, 4H), 5.10 (app. q, 2H), 7.36 (m, 5H).

Preparation 101

2-({1-[(Benzyloxy)carbonyl]cyclopentyl}methyl)pentanoic acid

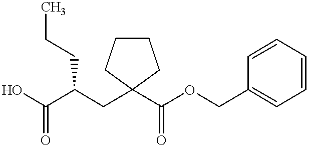

The product from Preparation 100 (430 mg, 1.15 mmol) was taken up in TFA (2 ml) under a nitrogen atmosphere and stirred for 16 h. The mixture was evaporated to dryness, and the residue then purified by column chromatography, using 95:5 DCM:MeOH to provide the title product (353, 97%); ¹HNMR (400 MHz, CDCl₃) 0.82 (t, 3H), 1.20-1.74 (m, 10H), 1.80 (dd, 1H), 2.04-2.13 (m, 3H), 2.24-2.41 (m, 1H), 5.10 (app q., 2H), 7.36 (m, 5H).

Preparation 102

Benzyl-1-(2-[{(5-ethyl-1,3,4-thiadiazol-2-yl)amino]carbonyl}pentyl)-cyclopentanecarboxylate

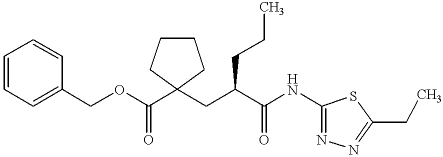

The product from Preparation 101 (353 mg, 1.11 mmol), 2-amino-5-ethyl-1,3,4-thiadiazole (ex Lancaster) (150 mg, 1.15 mmol), WSCDI (255 mg, 1.20 mmol), HOBt (173 mg, 1.20 mmol) and 4-methylmorpholine (0.24 mls, 1.20 mmol) were all mixed together in 5 mls of acetonitrile and stirred under nitrogen for 16 h at room temperature. After this time, the mixture was warmed to 50° C. for 3 h, and then at 80° C. for 3 h. The mixture was cooled to room temperature, evaporated, dissolved in EtOAc (10 mls) and washed with NaHCO₃ (10 mls). The organic layer was dried (MgSO₄) and evaporated to provide a gum which was purified by column chromatography to give the title product (430 mg, 90%); ¹HNMR (400 MHz, CDCl₃) 0.79 (t, 3H), 1.15-1.24 (m, 3H), 1.37 (t, 3H), 1.42-1.63 (m, 7H), 1.83 (dd, 1H), 2.00-2.20 (m, 3H), 2.42-2.51 (m, 1H), 2.97 (q, 2H), 5.01 (app. q, 2H), 7.30 (m, 5H).

NEP Assay

The Preparation and Assay of Soluble Neutral Endopeptidase (NEP) from Canine, Rat, Rabbit and Human Kidney Cortex

Soluble NEP is obtained from the kidney cortex and activity is assayed by measuring the rate of cleavage of the NEP substrate Abz-D-Arg-Arg-Leu-EDDnp to generate its fluorescent product, Abz-D-Arg-Arg.

Experimental Procedure:

1 Materials

All water is double de ionised.

1.1 Tissues:

| Tissues: | |
|---|---|
| Human Kidney | IIAM (Pennsylvania. U.S.A.) |
| Rat Kidney | In house tissue supply |
| Rabbit Kidney | In house tissue supply |
| Canine Kidney | In house tissue supply |

1.2 Homogenisation medium:

100 mM Mannitol and 20 mM Tris @ pH 7.1

2.42 g Tris (Fisher T/P630/60) is diluted in 1 liter of water and the pH adjusted to 7.1 using 6M HCl at room temperature. To this 18.22 g Mannitol (Sigma M-9546) is added.

1.3 Tris buffer (NEP buffer):

50 ml of 50 mM Tris pH 7.4 (Sigma T2663) is diluted in 950 ml of water.

1.4 Substrate (Abz-D-Arg-Arg-Leu-EDDnp):

Made to order from SNPE, and is stored as a powder at −20° C. A 2 mM stock is made by gently re-suspending the substrate in Tris buffer, this should not be vortexed or sonicated. 600 μl aliquots of the 2 mM stock are stored at −20 for up to one month. (Medeiros, M. A. S., Franca, M. S. F. et al., (1997), Brazilian Journal of Medical and Biological Research, 30, 1157-1162).

1.5 Total product:

Samples corresponding to 100% substrate to product conversion are included on the plate to enable the % substrate turnover to be determined. The total product is generated by incubating 1 ml of 2 mM substrate with 20 μl of enzyme stock for 24 hours at 37° C.

1.6 Stock solution:

A 300 μM stock of Phosphoramidon (Sigma R7385) is made up in NEP buffer and stored in 50 μl aliquots at −20.

1.7 Dimethyl sulphoxide (DMSO).

1.8 Magnesium Chloride-MgCl₂.6H₂O (Fisher M0600153).

1.9 Black 96 well flat bottom assay plates (Costar 3915).

1.10 Topseal A (Packard 6005185).

1.11 Centrifuge tubes

2 Specific Equipment

2.1 Sorvall RC-5B centrifuge (SS34 GSA rotor, pre-cooled to 4° C.).

2.2 Braun miniprimer mixer.

2.3 Beckman CS-6R centrifuge.

2.4 Fluostar galaxy.

2.5 Wesbart 1589 shaking incubator.

3 Methods

3.1 Tissue Preparation

3.2 Dog, rat, rabbit, and human NEP is obtained from the kidney cortex using a method adapted from Booth, A. G. & Kenny, A. J. (1974) Biochem. J. 142, 575-581.

3.3 Frozen kidneys are allowed to thaw at room temperature and the cortex is dissected away from the medulla.

3.4 The cortex is finely chopped and homogenised in approximately 10 volumes of homogenisation buffer (1.2) using a Braun miniprimer (2.2).

3.5 Magnesium chloride (1.8) (20.3 mg/gm tissue) is added to the homogenate and stirred in an ice-water bath for 15 minutes.

3.6 The homogenate is centrifuged at 1,500 g (3,820 rpm) for 12 minutes in a Beckman centrifuge (2.3) before removing the supernatant to a fresh centrifuge tube and discarding the pellet.

3.7 The supernatant is centrifuged at 15,000 g (12,100 rpm) for 12 minutes in a Sovall centrifuge (2.1) and the supernatant is discarded.

3.8 The pale pink layer on the top of the remaining pellet is removed and re-suspended in homogenisation buffer containing magnesium chloride (9 mg MgCl in 5 ml buffer per 1 g tissue).

3.9 The suspension is centrifuged at 2,200 g (4,630 rpm) for 12 minutes in a Beckman centrifuge (2.3) before discarding the pellet.

3.10 The supernatant is centrifuged at 15,000 g (12,100 rpm) for 12 minutes using the Sorvall centrifuge (2.1) and the supernatant is discarded.

3.11 The final pellet is resuspended in homogenisation buffer containing magnesium chloride (0.9 mg MgCl in 0.5 ml buffer per 1 g tissue). A homogenous suspension is obtained using a Braun miniprimer (2.2). This is then frozen down in 100 μl aliquots to be assayed for NEP activity.

4 Determination of NEP Activity

The activity of the previously aliquoted NEP is measured by its ability to cleave the NEP specific peptide substrate.

4.1 A 4% DMSO/NEP buffer solution is made (4 mls DMSO in 96 mls NEP buffer).

4.2 Substrate, total product, enzyme, and Phosphoramidon stocks are left on ice to thaw.

4.3 50 μl of 4% DMSO/NEP buffer solution is added to each well.

4.4 The 2 mM substrate stock is diluted 1:40 to make a 50 μM solution. 100 μl of 50 μM substrate is added to each well (final concentration 25 82 M).

4.5 50 μl of a range of enzyme dilutions is added to initiate the reaction (usually 1:100, 1:200, 1:400, 1:800, 1:1600, and 1:3200 are used). 50 μl of NEP buffer is added to blank wells.

4.6 The 2 mM total product is diluted 1:80 to make a 25 μM solution. 200 μl of 25 μM product is added to the first four wells of a new plate.

4.7 Plates are incubated at 37 degC in a shaking incubator for 60 minutes.

4.8 The 300 μM Phosphoramidon stock is diluted 1:100 to 300 μM. The reaction is stopped by the addition of 100 μl 300 nM Phosphoramidon and incubated at 37° C. in a shaking incubator for 20 minutes before being read on the Fluostar (ex320/em420).

5 NEP Inhibition Assays

5.1 Substrate, total product, enzyme and Phoshoramidon stocks are left on ice to thaw.

5.2 Compound stocks are made up in 100% DMSO and diluted 1:25 in NEP buffer to give a 4% DMSO solution. All further dilutions are carried out in a 4% DMSO solution (4 mls DMSO in 96 mls NEP buffer).

5.3 50 μl of compound in duplicate is added to the 96 well plate and 50 μl of 4% DMSO/NEP buffer is added to control and blank wells (see appendix for plate layout). Alternatively see appendix for robotic dilutions.

5.4 The 2 mM substrate stock is diluted 1:40 in NEP buffer to make a 50 μM solution (275 μl 2 mM substrate to 10.73 ml buffer is enough for 1 plate).

5.5 The enzyme stock diluted in NEP buffer (determined from activity checks).

5.6 The 2 mM total product stock is diluted 1:80 in NEP buffer to make a 25 μM solution. 200 μl is added to the first four wells of a separate plate.

5.7 The 300 μM Phosphoramidon stock is diluted 1:1000 to make a 300 nM stock (11 μl Phosphoramidon to 10.99 ml NEP buffer.

5.8 To each well in the 96 well plate the following is added:

TABLE
Reagents to be added to 96 well plate.

| | Compound/ | Tris | | NEP | Total |
|---|---|---|---|---|---|
| | DMSO | Buffer | Substrate | enzyme | product |
| Samples | 2 μl compound | 50 μl | 100 μl | 50 μl | None |
| Controls | 2 μl DMSO | 50 μl | 100 μl | 50 μl | None |
| Blanks | 2 μl DMSO | 100 μl | 100 μl | None | None |
| Totals | 2 μl DMSO | None | None | None | 200 μl |

5.9 The reaction is initiated by the addition of the NEP enzyme before incubating at 37° C. for 1 hour in a shaking incubator.

5.10 The reaction is stopped with 100 μl 300 nM Phosphoramidon and incubated at 37° C. for 20 minutes in a shaking incubator before being read on the Fluostar (ex320/em420).

6 Calculations

The activity of the NEP enzyme is determined in the presence and absence of compound and expressed as a percentage.

% Control activity (turnover of enzyme)=

$$\% Control activity ( turnover of enzyme ) = \frac{Mean FU of controls - Mean FU of blanks}{Mean FU of totals - Mean FU of blanks} \times 100$$

% Activity with inhibitor=

$$\% Activity with inhibitor = \frac{Mean FU of compound - Mean FU of blanks}{Mean FU of totals - Mean FU of blanks} \times 100$$

Activity expressed as % of control=

$$Activity expressed as \% of control = \frac{\% Activity with inhibitor}{\% Control activity} \times 100$$

A sigmoidal dose-response curve is fitted to the % activities (% of control) vs compound concentration and IC50 values calculated using LabStats fit-curve in Excel.

ACE Assay

The Preparation and Assay of Soluble Angiotensin Converting Enzyme (Ace), from Porcine and Human Kidney Cortex.

Soluble ACE activity is obtained from the kidney cortex and assayed by measuring the rate of cleavage of the ACE substrate Abz-Gly-p-nitro-Phe-Pro-OH to generate its fluorescent product, Abz-Gly.

1 Materials

All water is double de ionised.

1.1 Human Kidney: IIAM (Pennsylvania. U.S.A.) or UK Human Tissue Bank (UK HTB)

1.2 Porcine kidney ACE Sigma (A2580)

1.3 Homogenisation buffer-1

100 mM Mannitol and 20 mM Tris @ pH 7.1

2.42 g Tris (Fisher T/P630/60) is diluted in 1 liter of water and the pH adjusted to 7.1 using 6M HCl at room temperature. To this 18.22 g Mannitol (Sigma M-9546) is added.

1.4 Homogenisation buffer-2

100 mM Mannitol, 20 mM Tris @ pH7.1 and 10 mM MgCl_{2.}6H₂O (Fisher M0600/53)

To 500 ml of the homogenisation buffer 1 (1.4) 1.017 g of MgCl₂ is added.

1.5 Tris buffer (ACE buffer).

50 mM Tris and 300 mM NaCl @ pH 7.4

50 ml of 50 mM Tris pH 7.4 (Sigma T2663) and 17.52 g NaCl (Fisher S/3160/60) are made up to 1000 ml in water.

1.6 Substrate (Abz-D-Gly-p-nitro-Phe-Pro-OH) (Bachem M-1100)

ACE substrate is stored as a powder at −20° C. A 2 mM stock is made by gently re-suspending the substrate in ACE buffer, this must not be vortexed or sonicated. 400 μp aliquots of the 2 mM stock are stored at −20° C. for up to one month.

1.7 Total product

Samples corresponding to 100% substrate to product conversion are included on the plate to enable the % substrate turnover to be determined (see calculations).

The total product is generated by incubating 1 ml of 2 mM substrate with 20 μl of enzyme stock for 24 hours at 37° C.

1.8 Stop solution.

0.5M EDTA (Promega CAS[6081/92/6]) is diluted 1:250 in ACE buffer to make a 2 mM solution.

1.9 Dimethyl sulphoxide (DMSO).

1.10 Magnesium Chloride-MgCl₂.6H₂O (Fisher M0600/53).

1.11 Black 96 well flat bottom assay plates (Costar 3915 or Packard).

1.12 Topseal A (Packard 6005185).

1.13 Centrifuge tubes

2 Specific Equipment

2.1 Sorvall RC-5B centrifuge (SS34 GSA rotor, pre-cooled to 4° C.).

2.2 Braun miniprimer mixer.

2.3 Beckman CS-6R centrifuge.

2.4 BMG Fluostar Galaxy.

2.5 Wesbart 1589 shaking incubator.

3 Methods

3.1 Tissue Preparation

3.2 Human ACE is obtained from the kidney cortex using a method adapted from Booth, A. G. & Kenny, A. J. (1974) Biochem. J. 142, 575-581.

3.3 Frozen kidneys are allowed to thaw at room temperature and the cortex is dissected away from the medulla.

3.4 The cortex is finely chopped and homogenised in approximately 10 volumes of homogenisation buffer-1 (1.4) using a Braun miniprimer (2.2).

3.5 Magnesium chloride (1.11) (20.3 mg/gm tissue) is added to the homogenate and stirred in an ice-water bath for 15 minutes.

3.6 The homogenate is centrifuged at 1,500 g (3,820 rpm) for 12 minutes in a Beckman centrifuge (2.3) before removing the supernatant to a fresh centrifuge tube and discarding the pellet.

3.7 The supernatant is centrifuged at 15,000 g (12,100 rpm) for 12 minutes in a Sovall centrifuge (2.1) and the supernatant is discarded.

3.8 The pale pink layer on the top of the remaining pellet is removed and re-suspended in homogenisation buffer-2 (1.5) (5 ml buffer per 1 g tissue).

3.9 The suspension is centrifuged at 2,200 g (4,630 rpm) for 12 minutes in a Beckman centrifuge before discarding the pellet.

3.10 The supernatant is centrifuged at 15,000 g (12,100 rpm) for 12 minutes using the Sorvall centrifuge and the supernatant is discarded.

3.11 The final pellet is resuspended in homogenisation buffer-2 (0.5 ml buffer per 1 g tissue). A homogenous suspension is obtained using a Braun miniprimer. This is then frozen down in 10 0μl aliquots to be assayed for NEP activity.

4 Determination Of ACE Activity

The activity of the previously aliquoted ACE is measured by its ability to cleave the ACE specific peptide substrate.

Porcine ACE (1.2) is defrosted and resuspended in ACE buffer (1.6) at 0.004 U/μl, this is frozen down in 50 μl aliquots.

4.1 A 4% DMSO/ACE buffer solution is made (4 mls DMSO in 96 mls ACE buffer).

4.2 Substrate (1.7), total product (1.8) and enzyme (1.1, 1.2, 1.3), are left on ice to thaw.

4.3 50 μl of 4% DMSO/ACE buffer solution is added to each well.

4.4 The 2 mM substrate stock is diluted 1:100 to make a 20 μM solution. 100 μl of 20 μM substrate is added to each well (final concentration in the assay 10 μM).

4.5 50 μl of a range of enzyme dilutions is added to initiate the reaction (usually 1:100, 1:200, 1:400, 1:800, 1:1600, and 1:3200 are used). 50 μl of ACE buffer is added to blank wells.

4.6 The 2 mM total product is diluted 1:200 to make 10 μM solution. 200 μl 10 μM product is added to the first four wells of a new plate.

4.7 Plates are incubated at 37° C. in a shaking incubator for 60 minutes.

4.8 The enzyme reaction is stopped by the addition of 100 μl 2 mM EDTA in ACE buffer and incubated at 37° C. in a shaking incubator for 20 minutes before being read on the BMG Fluostar Galaxy (ex320/em420).

5 ACE Inhibition Assays

5.1 Substrate, total product, and enzyme stocks are left on ice to thaw.

5.2 Compound stocks are made up in 100% DMSO and diluted 1:25 in ACE buffer to give a 4% DMSO solution. All further dilutions are carried out in a 4% DMSO/ACE buffer solution (4 mls DMSO in 96 mls ACE buffer).

5.3 50 μl of compound, in duplicate, is added to the 96 well plate and 50 μl of 4% DMSO/ACE buffer is added to control and blank wells (see appendix-1 for plate layout).

5.4 Steps 5.2 and 5.3 can be carried out either by hand or using the Packard multiprobe robots (see appendix-2 for details)

5.5 The 2 mM substrate stock is diluted 1:100 in ACE buffer to make a 20 μM solution (10μM final concentration in the assay) (110 μM of 2 mM substrate added to 10.89 ml buffer is enough for 1 plate).

5.6 The enzyme stock is diluted in ACE buffer, as determined from activity checks (4.0).

5.7 The 2 mM total product stock is diluted 1:200 in ACE buffer to make a 10 μM solution. 200μl is added to the first four wells of a separate plate.

5.8 The 0.5 mM EDTA stock is diluted 1:250 to make a 2 mM stock (44 μl EDTA to 10.96 ml ACE buffer).

5.9 To each well of the 96 well plate the following reagents are added:

TABLE 1
Reagents added to 96 well plate.

| | Compound/ | Tris | | ACE | Total |
|---|---|---|---|---|---|
| | DMSO | Buffer | Substrate | enzyme | product |
| Samples | 2 μl compound | 50 μl | 100 μl | 50 μl | None |
| Controls | 2 μl DMSO | 50 μl | 100 μl | 50 μl | None |
| Blanks | 2 μl DMSO | 100 μl | 100 μl | None | None |
| Totals | 2 μl DMSO | None | None | None | 200 μl |

5.10 50 μl of the highest concentration of each compound used in the assay is added in duplicate to the same 96 well plate as the totals (5.7). 150 μl of ACE buffer is added to determine any compound fluorescence.

5.11 The reaction is initiated by the addition of the ACE enzyme before incubating at 37° C. for 1 hour in a shaking incubator.

5.12 The reaction is stopped by the addition of 100 μl 2 mM EDTA and incubated at 37° C. for 20 minutes in a shaking incubator, before being read on the BMG Fluostar Galaxy (ex320/em420).

6 Calculations

The activity of the ACE enzyme is determined in the presence and absence of compound and expressed as a percentage. (FU=Fluorescence units)

(i) % Control activity (turnover of enzyme)=

$$\% Control activity ( turnover of enzyme ) = \frac{Mean FU of controls - Mean FU of blanks}{Mean FU of totals - Mean FU of blanks} \times 100 \qquad ( i )$$

(ii) % Activity with inhibitor=

$$\% Activity with inhibitor = \frac{Mean FU of compound - Mean FU of blanks}{Mean FU of totals - Mean FU of blanks} \times 100 \qquad ( ii )$$

(iii) Activity expressed as % of control=

$$Activity expressed as \% of control = \frac{\% Activity with inhibitor}{\% Control activity} \times 100 \\ or \\ \frac{Mean FU of compound - Mean FU of blanks}{Mean FU of controls - Mean FU of blanks} \times 100 \qquad ( iii )$$

(iv) % Inhibition=100−% control

(v) For fluorescent compounds the mean FU of blanks containing compound (5.10) is deducted from the mean FU of compound values used to calculate the % Activity.

A sigmoidal dose-response curve is fitted to the % activities (% of control) vs compound concentration and IC₅₀ values calculated using LabStats fit-curve in Excel.

The specific examples herein had an IC50 against NEP of less than 5000 nM.

In addition many of the examples tested had a selectivity for NEP over ACE of at least 300 fold.

Animal Model of arousal response

We have developed an animal model that mimics the physiological arousal response observed during female sexual arousal and directly reflects the clinical data obtained in human volunteers. The model uses Laser Doppler technologies to record small changes in vaginal and clitoral blood flow induced by pelvic nerve stimulation or vasoactive neurotransmitters. During sexual arousal there is an increase in genital blood flow resulting from increased innervation from the pelvic nerve. This increase in genital blood flow leads to increased genital lubribriation and genital sensitivity which are associated with sexual arousal. The major cause of FSAD is decreased genital blood flow and this manifests itself as reduced vaginal, labial and clitoral engorgement. Treatment of women with FSAD is achievable by restoration of the normal sexual arousal response. This can be achieved by enhancing genital blood flow.

The pelvic nerve-stimulated increase in vaginal and clitoral blood flow, observed in the animal model, represents the endogenous vascular effects observed during female sexual arousal. Therefore this model can be used to firstly, identify the mechanisms involved in the regulation of vaginal and clitoral blood flow and secondly, use the model to validate novel approaches for the enhancement of genital blood flow.

The title product from Example 29 was administered using this animal model according to the following protocol to show an enhancement in pelvic nerve stimulated increases in genital blood flow in the rabbit.

Two routes of administration were studied: a) intravenous administration and b) topical administration. Both studies used the anesthatised rabbit model according to the following protocol.

Methods

Anaesthetic Protocol

Female New Zealand rabbits (˜2.5 kg) were pre-medicated with a combination of Medetomidine (Domitor®) 0.5 ml/kg i.m., and Ketamine (Vetalar®) 0.25 ml/kg i.m. whilst maintaining oxygen intake via a face mask. The rabbits were tracheotomised using a Portex™ uncuffed endotracheal tube 3 ID, connected to ventilator and maintained a ventilation rate of 30-40 breaths per minute, with an approximate tidal volume of 18-20 ml, and a maximum airway pressure of 10 cm H₂O. Anaesthesia was then switched to Isoflurane and ventilation continued with O₂ at 2 l/min. The right marginal ear vein was cannulated using a 23 G or 24 G catheter, and Lactated Ringer solution perfused at 0.5 ml/min. The rabbit was maintained at 3% Isoflurane during invasive surgery, dropping to 2% for maintenance anaesthesia.

Stimulation of the Pelvic Nerve

A ventral midline incision was made into the abdominal cavity. The incision was about 5 cm in length just above the pubis. The fat and muscle was bluntly dissected away to reveal the hypogastric nerve which runs down the body cavity. It was essential to keep close to the side curve of the pubis wall in order to avoid damaging the femoral vein and artery which lie above the pubis. The sciatic and pelvic nerves lie deeper and were located after further dissection on the dorsal side of the rabbit. Once the sciatic nerve is identified, the pelvic nerve was easily located. The term pelvic nerve is loosely applied; anatomy books on the subject fail to identify the nerves in sufficient detail. However, stimulation of the nerve causes an increase in vaginal and clitoral blood flow, and innervation of the pelvic region. The pelvic nerve was freed away from surrounding tissue and a Harvard bipolar stimulating electrode was placed around the nerve. The nerve was slightly lifted to give some tension, then the electrode was secured in position. Approximately 1 ml of light paraffin oil was placed around the nerve and electrode. This acts as a protective lubricant to the nerve and prevents blood contamination of the electrode. The electrode was connected to a Grass S88 Stimulator. The pelvic nerve was stimulated using the following parameters: −5 V, pulse width 0.5 ms, duration of stimulus 10 seconds and a frequency range of 2 to 16 Hz. Reproducible responses were obtained when the nerve was stimulated every 15-20 minutes.

A frequency response curve was determined at the start of each experiment in order to determine the optimum frequency to use as a sub-maximal response, normally 4 Hz. The compound(s) to be tested were infused, via the femoral vein, using a Harvard 22 infusion pump allowing a continuous 15 minute stimulation cycle.

Positioning of the Laser Doppler Probes

A ventral midline incision was made, at the caudal end of the pubis, to expose the pubic area. Remove any connective tissue, and expose the tunica of the clitoris, ensuring that the wall is free from small blood vessels. The external vaginal wall was also exposed by removing any connective tissue. One laser Doppler flow probe was inserted 3cm into the vagina, so that half the probe shaft is still visible. A second probe was positioned so that it lies just above the external clitoral wall. The position of these probes was then adjusted until a signal was obtained. A second probe was placed just above the surface of a blood vessel on the external vaginal wall. Both probes were clamped in position.

Vaginal and clitoral blood flow was recorded either as numbers directly from the Flowmeter using Po-ne-mah data acquisition software (Ponemah Physiology Platform, Gould Instrument Systems Inc), or indirectly from Gould chart recorder trace. Calibration is set at the beginning of the experiment (0-125 ml/min/100 g tissue).

Administration of Inhibitors

a) Intravenous administration

Cannulation of Blood Vessels

The left groin area of the rabbit was shaved and a vertical incision was made approximately 5 cm in length along the thigh. The femoral vein and artery were exposed, isolated and then cannulated with a PVC catheter (17 G) for the infusion of drugs and compounds. Cannulation was repeated for the femoral artery, inserting the catheter to a depth of 10 cm to ensure that the catheter reached the abdominal aorta. This arterial catheter was linked to a Gould system to record blood pressure. Samples for blood gas analysis were also be taken via the arterial catheter. Systolic and diastolic pressures were measured, and the mean arterial pressure calculated using the formula (diastolic×2+systolic)+3. Heart rate was measured via the pulse oxymeter and Po-ne-mah data acquisition software system (Ponemah Physiology Platform, Gould Instrument Systems Inc).

The title compound form Example 29 was made up in saline or 5% glucose solution (200 μl 50% glucose in 1.8 ml water for injection). The inhibitor and vehicle controls were infused using a Harvard 22 pump, infusing at 500 μl/min via a 3-way tap into the femoral vein. After the infusion, the catheter was flushed with heparinised saline (Hepsaline) so that no NEP inhibitor was left in the catheter.

b) Topical administration of Inhibitors

A topical formulation was prepared mixing the product from Example 29 at 90% saturation in 50% propylene glycol/50% water. The mixture was made viscous with carboxymethyl cellulose (CMC) giving a final concentration of approximately 2.5 mg/ml. The administration of 0.2 ml provides a maximal dose of 0.5 mg.

This formulation was applied topically to the internal vaginal wall via an in house designed applicator. Basically, pieces of tubing (ID 3 mm, OD 4 mm) 10 cm in length were cut and attached to a 1 ml syringes. The syringes were each filled with a control gel (containing no active ingredient) or the formulation described above. The tubing was inserted 2 cm into the vagina and 0.2 ml of the gel was gently injected to avoid disturbing the laser Doppler probe. The addition of gel caused no major distension to the vagina and there was no excessive leakage of the gels from within the vagina during non-stimulated or stimulated periods.

Results and Discussion

Animal model of sexual arousal

We have developed a robust reproducible model of the physiology of sexual arousal. Using this anaesthetised rabbit model, we are capable of measuring small changes in genital blood flow using Laser Doppler technology. Stimulation of the pelvic nerve is used to simulate the neuronal effects of sexual arousal.

FSAD is associated with and may result from reduced genital blood flow.

Our results demonstrate that the title compound of Example 29 significantly enhanced pelvic nerve stimulated increases in genital blood flow at clinically relevant doses. This enhancement is seen with both intravenous and topical administration.

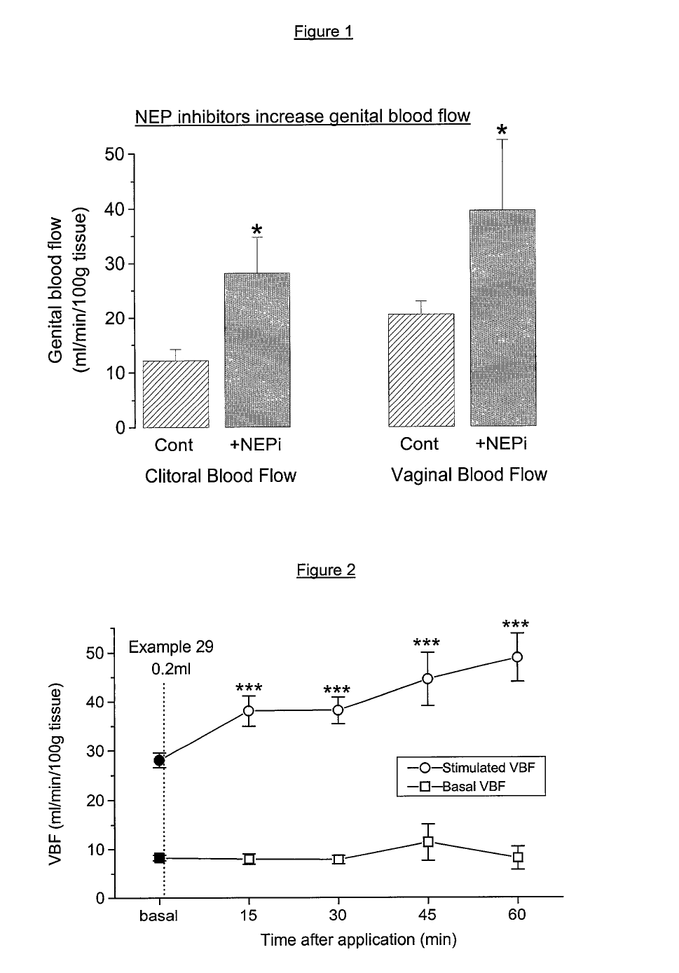

FIG. 1 shows the effect on vaginal and clitoral blood flow of intravenous administration of the title product from Example 29. Intravenous administration enhanced pelvic nerve stimulated (PNS) increases in genital blood flow in the anaesthetised rabbit model of sexual arousal. Repetitive PNS at 15 minute intervals induced reproducible increases in genital blood flow (Hatched Bars). Intravenous administration of the title product from Example 29 (Grey bar) enhanced the peak increase in clitoral and vaginal blood flow induced by submaximal stimulation frequencies (eg 4 Hz) compared to increases observed during time matched control stimulations or vehicle controls (Hatched bar). The following simultaneous enhancements were observed following a 1.0 mg/kg iv bolus infusion—a 131% increase in clitoral and a 92% increase in vaginal blood flow (n=3). Data expressed as mean±sem; all changes were monitored using laser Doppler technologies.

FIG. 2 shows the effect on vaginal blood flow over time of administering the title product from Example 29 topically. There was no observable change to non-stimulated/basal vaginal blood flow, neither were any changes observed on stimulated vaginal blood flow after the initial insertion of the tubing or application of the 0.2 ml of the vehicle gel.

Repetitive pelvic nerve stimulation at submaximal stimulation frequencies at 15 minute intervals induces reproducible increases in vaginal blood flow (filled circle). Intravaginal application of a set concentration of the tile product from Example 29 (0.2 mg/ml) enhanced the peak increase in vaginal blood flow (open circles) compared to the mean control flow increases. The title product from Example 29 had no effect on basal (non-stimulated) vaginal blood flow (open squares) compared to control flow (filled square). All changes were monitored using laser Doppler technologies and data is expressed as mean+s.e.mean (n=4), *** P<0.001 Student's t-test.

This study demonstrates that the title product from Example 29, when applied topically to the vagina significantly enhanced pelvic nerve-stimulated increases in vaginal blood flow. The degree of enhancement is comparable to increases observed after intravenous infusion of the compound. Interestingly, the enhancement occurred at free plasma concentration of the title product from Example 29 which would not be expected to cause a potentiation of vaginal blood flow.

In conclusion, this study demonstrates that intravaginal, topical application of the compounds of the invention enhances pelvic nerve stimulated increases in vaginal blood flow.

1. A method of treating female sexual dysfunction comprising administering a therapeutically effective amount of a compound of formula (I), pharmaceutically acceptable salts, solvates, polymorphs or prodrugs thereof: 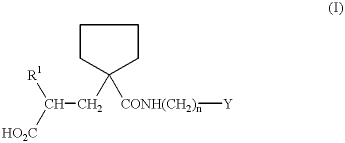
wherein
R¹ is C₁₋₆alkyl which may be substituted by one or more substituents, which may be the same or different, selected from the list: halo, hydroxy, C₁₋₆ alkoxy, C₂₋₆hydroxyalkoxy, C₁₋₆alkoxy(C₁₋₆alkoxy), C₃₋₇cycloalkyl, C₃₋₇cycloalkenyl, aryl, aryloxy, (C₁₋₄alkoxy)aryloxy, heterocyclyl, heterocyclyloxy, —NR²R³, —NR⁴COR⁵, —NR⁴S₂R⁵, —CONR²R³, —S(O)ₚR⁶, —COR⁷ and —CO₂(C₁₋₄alkyl); or R¹ is C₃₋₇cycloalkyl, aryl or heterocyclyl, each of which may be substituted by one or more substituents from said list, which substituents may be the same or different, which list further includes C₁₋₆alkyl; or R¹ is C₁₋₆ alkoxy, —NR²R³ or —NR⁴SO₂R⁵;
wherein
R² and R³ are each independently H, C₁₋₄alkyl, C₃₋₇cycloalkyl (optionally substituted by hydroxy or C₁₋₄alkoxy), aryl, (C₁₋₄alkyl)aryl, C₁₋₆alkoxyaryl or heterocyclyl; or R² and R³ together with the nitrogen to which they are attached form a pyrrolidinyl, piperidino, morpholino, piperazinyl or N—(C₁₋₄ alkyl)piperazinyl group;
R⁴ is H or C₁₋₄alkyl;
R⁵ is C₁₋₄alkyl, CF₃, aryl, (C₁₋₄alkyl)aryl, (C₁₋₄alkoxy)aryl, heterocyclyl, C₁₋₄alkoxy or —NR²R³ wherein R² and R³ are as previously defined;
R⁶ is C₁₋₄alkyl, aryl, heterocyclyl or NR²R³ wherein R² and R³ are as previously defined; and
R⁷ is C₁₋₄alkyl, C₃₋₇cycloalkyl, aryl or heterocyclyl; p is 0, 1, 2 or 3;
n is 0, 1 or 2;
the —(CH₂ )ₙ— linkage is optionally substituted by C₁₋₄alkyl, C₁₋₄alkyl substituted with one or more fluoro groups or phenyl, C₁₋₄alkoxy, hydroxy, hydroxy(C_{1 3}alkyl), C₃₋₇cycloalkyl, aryl or heterocyclyl;
Y is the group 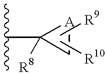
wherein A is —(CH₂)_{q}— where q is 1, 2, 3 or 4 to complete a 3 to 7 membered carbocyclic ring which may be saturated or unsaturated; R⁸ is H, C₁₋₆alkyl, —CH₂OH, phenyl, phenyl(C₁₋₄alkyl) or CONR¹¹R¹²; R⁹ and R¹⁰ are each independently H, —CH₂OH, —C(O)NR¹¹R¹², C₁₋₆alkyl, phenyl (optionally substituted by C₁₋₄alkyl, halo or C₁₋₄alkoxy or phenyl(C₁₋₄alkyl) wherein the phenyl group is optionally substituted by C₁₋₄alkyl, halo or C₁₋₄alkoxy, or R⁹ and R¹⁰ together form a dioxolane; R¹¹ and R¹² which may be the same or different are H, C₁₋₄alkyl, R¹³ or S(O)ᵣR¹³, where r is 0, 1 or 2 and R¹³ is phenyl optionally substituted by C₁₋₄alkyl or phenylC₁₋₄alkyl wherein the phenyl is optionally substituted by C₁₋₄alkyl; or
Y is the group, —C(O)NR¹¹R¹² wherein R¹¹ and R¹² are as previously defined except that R¹¹ and R¹² are not both H; or
Y is the group, 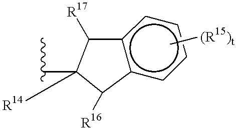
wherein R¹⁴ is H, CH₂OH, or C(O)NR¹¹R¹² wherein R¹¹ and R¹² are as previously defined; when present R¹⁵, which may be the same or different to any other R¹⁵, is OH, C₁₋₄alkyl, C₁₋₄alkoxy, halo or CF₃; t is 0, 1, 2, 3 or 4; and R¹⁶ and R¹⁷ are independently H or C₁₋₄alkyl; or
Y is the group 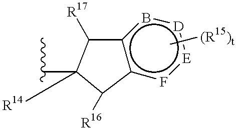
wherein one or two of B, D, E or F is a nitrogen, the others being carbon; and R¹⁴ to R¹⁷ and t are as previously defined; or
Y is an optionally substituted 5-7 membered heterocyclic ring, which may be saturated, unsaturated or aromatic and contains a nitrogen, oxygen or sulphur and optionally one, two or three further nitrogen atoms in the ring and which may be optionally benzofused and optionally substituted by:
C₁₋₆ alkoxy; hydroxy; oxo; amino; mono or di-(C₁₋₄alkyl)amino; C₁₋₄alkanoylamino; or
C₁₋₆alkyl which may be substituted by one or more substituents, which may be the same or different, selected from the list: C₁₋₆alkoxy, C₁₆ haloalkoxy, C₁₋₆alkylthio, halogen, C₃₋₇cycloalkyl, heterocyclyl or phenyl; or
C₃₋₇cycloalkyl, aryl or heterocyclyl, each of which may be substituted by one or more substituents, which may be the same or different, selected from the list: C₁₋₆alkyl, C₁₋₆alkoxy, C₁₋₆ haloalkoxy, C₁₋₆alkylthio, halogen, C₃₋₇cycloalkyl, heterocyclyl or phenyl;
wherein when there is an oxo substitution on the heterocyclic ring, the ring only contains one or two nitrogen atoms and the oxo substitution is adjacent a nitrogen atom in the ring; or
Y is —NR¹⁸S(O)ᵤR¹⁹, wherein R¹⁸ is H or C 4alkyl; R¹⁹ is aryl, arylC₁₋₄alkyl or heterocyclyl; and u is 0, 1, 2 or 3.

2. A compound of formula (I), pharmaceutically acceptable salts, solvates, polymorphs or prodrugs thereof, wherein R¹, n and Y are as defined in claim 1 with the proviso that Y is not the group —C(O)NR¹¹R¹² and when R¹ is propyl or phenylethyl, R¹⁴ is not —CH₂OH.

3. A compound of formula (I), pharmaceutically acceptable salts, solvates, polymorphs or prodrugs thereof, wherein R¹, n and Y are as defined in claim 1 with the proviso that Y is not the group —C(O)NR¹¹R¹² and R¹⁴ is not H or —CH₂OH.

4. A compound according to claim 2, pharmaceutically acceptable salts, solvates, polymorphs or prodrugs thereof, wherein R¹ is C₁₋₆alkyl, C₁₋₆alkoxy, C₁₋₆alkoxy(C₁₋₃)alkyl, C₁₋₆alkoxyC₁₋₆alkoxyC₁₋₃alkyl or C₁₋₆alkyl substituted with aryl.

5. A compound according to claim 4, pharmaceutically acceptable salts, solvates, polymorphs or prodrugs thereof, wherein R¹ is C₁₋₆alkyl, C₁₋₆alkoxy, C₁₋₆alkoxy(C₁₋₃)alkyl or C₁₋₆alkoxyC₁₋₆alkoxyC₁₋₃alkyl.

6. A compound according to claim 5, pharmaceutically acceptable salts, solvates, polymorphs or prodrugs thereof, wherein R¹ is C₁₋₄alkyl or C₁₋₆alkoxy(C₁₋₃)alkyl.

7. A compound according to claim 2, pharmaceutically acceptable salts, solvates, polymorphs or prodrugs thereof, wherein when Y is the group 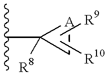
and the carbocyclic ring is fully saturated, then preferably one of R⁹ or R¹⁰ is —CH₂OH; —C(O)NR¹¹R¹²; C₁₋₆alkyl; phenyl optionally substituted by C₁₋₄alkyl; or phenyl(C₁₋₄alkyl) wherein the phenyl group is optionally substituted by C₁₋₄alkyl.

8. A compound according to claim 7, pharmaceutically acceptable salts, solvates, polymorphs or prodrugs thereof, wherein the carbocyclic ring is 5, 6 or 7 membered wherein one of R⁹ or R¹⁰, is —C(O)NR¹¹R¹², with the other being C₁₋₆alkyl; phenyl optionally substituted by C₁₋₄alkyl; or phenyl(C₁₋₄alkyl) wherein the phenyl group is optionally substituted by C₁₋₄alkyl.

9. A compound according to claim 7, pharmaceutically acceptable salts, solvates, polymorphs or prodrugs thereof, wherein R⁹ and R¹⁰ are attached to adjacent carbon atoms in the ring.

10. A compound according to claim 7 pharmaceutically acceptable salts, solvates, polymorphs or prodrugs thereof, wherein R⁸ is CH₂OH.

11. A compound according to claim 2, pharmaceutically acceptable salts, solvates, polymorphs or prodrugs thereof, wherein when Y is the group —NR¹⁸S(O)ᵤR¹⁹, preferably R¹⁸ is H.

12. A compound according to claim 2, pharmaceutically acceptable salts, solvates, polymorphs or prodrugs thereof, wherein R¹⁹ is benzyl or phenyl.

13. A compound according to claim 2, pharmaceutically acceptable salts, solvates, polymorphs or prodrugs thereof, wherein u is 2.

14. A compound according to claim 2, pharmaceutically acceptable salts, solvates, polymorphs or prodrugs thereof, wherein Y is an optionally substituted 5-7 membered heterocyclic ring.

15. A compound according to claim 14, pharmaceutically acceptable salts, solvates, polymorphs or prodrugs thereof, wherein the 5-7 membered heterocyclic ring is an optionally substituted aromatic ring.

16. A compound according to claim 15, pharmaceutically acceptable salts, solvates, polymorphs or prodrugs thereof, wherein said aromatic ring is pyridyl, pyrazinyl, pyrimidinyl, pyridazinyl, pyrazolyl, triazolyl, tetrazolyl, oxadiazolyl, thiazolyl, thiadiazolyl, oxazolyl, isoxazolyl, indolyl, isoindolinyl, quinolyl, isoquinolyl, pyridonyl, quinoxalinyl or quinazolinyl each of which may be substituted as defined in claim 1.

17. A compound according to claim 16, pharmaceutically acceptable salts, solvates, polymorphs or prodrugs thereof, wherein the aromatic ring is oxadiazole, pyridone or thiadiazole each of which may be substituted as defined in claim 1.

18. A compound according to claim 17, pharmaceutically acceptable salts, solvates, polymorphs or prodrugs thereof, wherein the aromatic ring is 1,2,5-oxadiazole, 1,3,4-oxadiazole, 2-pyridone or 1,3,4-thiadiazole each of which may be substituted as defined in claim 1.

19. A compound according to claim 14 , pharmaceutically acceptable salts, solvates, polymorphs or prodrugs thereof, wherein the 5-7 membered heterocyclic ring is substituted by one or more C₁₋₆alkyl, phenyl or phenylC₁₋₄alkyl.

20. A compound according to claim 19, pharmaceutically acceptable salts, solvates, polymorphs or prodrugs thereof, wherein the 5-7 membered heterocyclic ring is substituted by C₁₋₄alkyl or benzyl.

21. A compound according to claim 17, pharmaceutically acceptable salts, solvates, polymorphs or prodrugs thereof, wherein when Y is a pyridone said pyridone is N-substituted pyridone.

22. A compound according to claim 14, pharmaceutically acceptable salts, solvates, polymorphs or prodrugs thereof, wherein Y is a lactam linked at the nitrogen.

23. A compound according to any claim 2, pharmaceutically acceptable salts, solvates, polymorphs or prodrugs thereof, wherein Y is 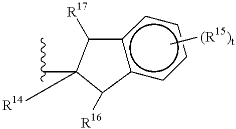
wherein R¹⁴ is CH₂OH or C(O)NR¹¹R¹²

24. A compound according to claim 2, pharmaceutically acceptable salts, solvates, polymorphs or prodrugs thereof, wherein R¹⁶ and R¹⁷ are hydrogen.

25. A compound according to claim 2, pharmaceutically acceptable salts, solvates, polymorphs or prodrugs thereof, wherein t is 0.

26. A compound of formula Ie, pharmaceutically acceptable salts, solvates, polymorphs or prodrugs thereof, 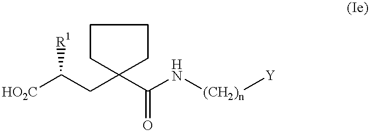
wherein R¹, Y and n are as defined in claim 2.

27. A compound, pharmaceutically acceptable salts, solvates, polymorphs or prodrugs thereof, selected from the group consisting of:
2-[(1-{[(1-benzyl-6-oxo-1 ,6-dihydro-3-pyridinyl)amino]carbonyl}cyclopentyl)-methyl]-4-methoxybutanoic acid;
2-{[1-({[3-(2-oxo-1-pyrrolidinyl)propyl]amino}carbonylcyclopentyl]-methyl}-4-phenylbutanoic acid);
(+)-2-{[1-({[2-(hydroxymethyl)-2,3-dihydro-1H-inden-2-yl]amino}carbonyl)cyclopentyl]methyl}-4-phenylbutanoic acid;
2-[(1-{[(5-methyl-1,3,4-thiadiazol-2-yl)amino]carbonyl}cyclopentyl)methyl]-4-phenylbutanoic acid;
cis-3-(2-methoxyethoxy)-2-[(1-{[(4-{[(phenylsulfonyl)amino]carbonyl}cyclohexyl)-amino]carbonyl}cyclopentyl)methyl]propanoic acid;
(+)-2-{[1-({[2-(hydroxymethyl)-2,3-dihydro-1H-inden-2-yl]amino}carbonyl)cyclopentyl]-methyl}pentanoic acid;
(2R)-2-[(1-{[(5-ethyl-1,3,4-thiadiazol-2-yl)amino]carbonyl}cyclopentyl)-methyl]pentanoic acid or (−)-2-[(1-{[(5-ethyl-1,3,4-thiadiazol-2-yl)amino]carbonyl}cyclopentyl)-methyl]pentanoic acid;
(2S)-2-[(1-{[(5-ethyl-1,3,4-thiadiazol-2-yl)amino]carbonyl}cyclopentyl)-methyl]pentanoic acid or (+)-2-[(1-{[(5-ethyl-1,3,4-thiadiazol-2-yl)amino]carbonyl}cyclopentyl)-methyl]pentanoic acid;
2-({1-[(3-benzylanilino)carbonyl]cyclopentyl}methyl)pentanoic acid;
2-[(1-{[(1-benzyl-6-oxo-1 ,6-dihydro-3-pyridinyl)amino]carbonyl}cyclopentyl)-methyl]pentanoic acid;
2-{[1-({[(1R,3S,4R)-4-(aminocarbonyl)-3-butylcyclohexyl]amino}carbonyl)-cyclopentyl]methyl}pentanoic acid;
trans-3-[1-({[2-(4-chlorophenyl)cyclopropyl]amino}carbonyl)cyclopentyl]-2-(methoxymethyl)propanoic acid;
trans-3-[1-({[2-(4-methoxyphenyl)cyclopropyl]amino}carbonyl)cyclopentyl]-2-(methoxyethyl)propanoic acid;
trans-3-[l -({[2-pentylcyclopropyl]amino}carbonyl)cyclopentyl]-2-(methoxyethyl)propanoic acid;
3-[1-({[5-benzyl-[1,3,4]-thiadiazol-2-yl]amino}carbonyl)cyclopentyl]-2-(methoxyethyl)propanoic acid;
3-[1-({[4-butylpyridin-2-yl]amino}carbonyl)cyclopentyl]-2-(methoxyethyl)propanoic acid;
3-[1-({[4-phenylpyridin-2-yl]amino}carbonyl)cyclopentyl]-2-(methoxyethyl)propanoic acid
3-[1-({[1-hydroxymethyl-3-phenylcyclopentyl]amino}carbonyl)cyclopentyl]-2-(methoxyethyl)propanoic acid;
2-{[1-({[2-(hydroxymethyl)-2,3-dihydro-1H-inden-2-yl]amino}carbonyl)-cyclopentyl]methyl}-4-methoxybutanoic acid
trans-3-[1-({[2-phenylcyclopropyl]amino}carbonyl)cyclopentyl]-2-(methoxyethyl)propanoic acid;
(R)-2-{[l-({[2-(hydroxymethyl)-2,3-dihydro-1H-inden-2-yl]amino}carbonyl)-cyclopentyl]methyl}-4-methoxybutanoic acid; and
(S)-2-{[1-({[2-(hydroxymethyl)-2,3-dihydro-1H-inden-2-yl]amino}carbonyl)-cyclopentyl]methyl}-4-methoxybutanoic acid.

28. The method according to claim 1 wherein the female sexual dysfunction treated includes at least female sexual arousal dysfunction (FSAD).

29. The method according to claim 1 wherein the medicament is administered systemically.

30. The method according to claim 1 wherein the medicament is administered orally.

31. A method of treatment or prophylaxis of a condition for which a beneficial therapeutic response can be obtained by the inhibition of neutral endopeptidase comprising administration of a therapeutically effective amount of a compound as defined in claim 2.

32. A medicine comprising the compound of claim 2.

33. A pharmaceutical formulation including a compound as defined in claim 2 together with a pharmaceutically acceptable excipient.

34. A method for the treatment or prophylaxis of female sexual dysfunction including administering to the patient a therapeutically effective amount of a compound as defined in claim 2.

35. A female sexual dysfunction pharmaceutical formulation including a therapeutically effective amount of a compound as defined in claim 2 together with a pharmaceutically acceptable excipient.

36. A process for preparing a compound of formula I or salts thereof 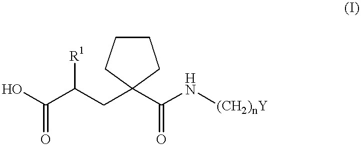
wherein R¹, n and Y are as defined in any one of claims 2 to 27, comprising the steps of:
a) reacting a compound of formula II 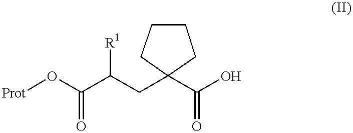
wherein Prot is a suitable protecting group, with a compound of formula Y(CH₂)ₙNH₂ (III), to give a compound of formula IV, 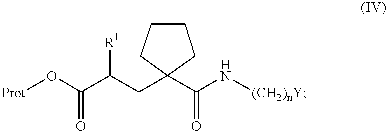
then
b) reacting the compound of formula IV under suitable deprotecting conditions to give the compound of formula l; then
c) optionally forming a salt.

37. A compound of formula IV 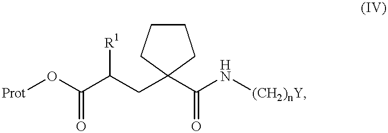
wherein R¹, n, and Y are as defined in claim 2 and wherein Prot is a protecting group.
